(19) United States
(12) Patent Application Publication
(10) Pub. No.: US 2026/0053543 A1
(43) Pub. Date: Feb. 26, 2026

(54) MINIMALLY-INVASIVE SLICING

(71) Applicant: TAG DREAM MEDICAL LTD., Yokne'am llit (IL)

(72) Inventors: Yuval PERETS, Moshav Beit Shearim (IL); Elazar LIBERMAN, Kibbutz Harduf (IL); Dan NABEL, Kiryat Tivon (IL); Dan MICHAEL, Moshav Beit Shearim (IL); Yaron TAL, Netanya (IL)

(73) Assignee: TAG DREAM MEDICAL LTD., Yokne'am llit (IL)

(21) Appl. No.: 19/140,911

(22) PCT Filed: Oct. 31, 2024
(86) PCT No.: PCT/IL2024/051047

§ 371 Date: Jun. 18, 2025

Related U.S. Application Data
(60) Provisional application No. 63/547,014, filed on Nov. 2, 2023.

(51) Int. Cl. A61B 18/12; A61B 18/00; A61B 18/14; A61B 18/16

(52) U.S. Cl. CPC A61B 18/1206; A61B 18/16; A61B 2018/00083; A61B 2018/00184; A61B 2018/00601; A61B 2018/1435; A61B 2018/144

(57) ABSTRACT

A tissue removal apparatus (20) is provided that includes a retrieval bag (30) having an opening (32) for receiving a resected tissue specimen (22) within a patient's body (24). An electrosurgical slicing wire (34) includes a slicing segment (36) detachably coupled to a wall (38) of the retrieval bag (30), and a pulling segment (40) free of the wall (38) and extending out of the opening (32). The slicing segment (36) winds around a central longitudinal axis (46) of the retrieval bag (30) at varying locations along the central longitudinal axis (46). Pulling the pulling segment (40), during application of RF electrosurgical power when the resected tissue specimen (22) is within the retrieval bag (30), detaches successive longitudinal portions of the slicing segment (36) from the wall (38) and slices the resected tissue specimen (22) within the retrieval bag (30). Other embodiments are also described.

### CROSS-REFERENCE TO RELATED APPLICATIONS

The present application claims priority from U.S. Provisional Application 63/547,014, filed Nov. 2, 2023, which is assigned to the assignee of the present application and incorporated herein by reference in its entirety.

### FIELD OF THE APPLICATION

The present invention relates generally to minimally-invasive surgical tools and methods, and specifically to minimally-invasive surgical tools methods for slicing and removing tissue from a patient's body.

### BACKGROUND OF THE APPLICATION

Hysterectomy, the surgical removal of the uterus, is typically performed in a minimally-invasive procedure, either laparoscopically or vaginally. The steps of a minimally-invasive hysterectomy include uterus dissection (the actual hysterectomy), the insertion of a tissue containment system into the abdominal cavity, the insertion of the dissected tissue specimen into a retrieval bag, the morcellation of the tissue specimen within the retrieval bag, and the extraction of the morcellated pieces of tissue from the retrieval bag via a laparoscopic or a vaginal port. Morcellation of the tissue specimen is conventionally performed either using a power morcellator or manually. Similar techniques are used for removing other large tissue specimens from the body, such as organs or portions of organs, e.g., kidneys, portions of livers, and fibroids.

### SUMMARY OF THE APPLICATION

In some embodiments of the present invention, a tissue removal apparatus is provided for removing a resected tissue specimen from a body of a patient. For example, the resected tissue specimen may be all or a portion of an organ, such as a uterus, a spleen, a kidney, or a lung, or another tissue, such as a fibroid. The tissue removal apparatus comprises a retrieval bag having an opening for receiving the resected tissue specimen within the patient's body, an electrosurgical slicing wire, and a return electrode. The electrosurgical slicing wire includes a slicing segment detachably coupled to a wall of the retrieval bag, and a pulling segment free of the wall of the retrieval bag and extending out of the opening of the retrieval bag. The return electrode is configured to be brought into electrical contact with the resected tissue specimen when the resected tissue specimen is within the retrieval bag.

The tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.

For some applications, the tissue removal apparatus comprises exactly one electrosurgical slicing wire that includes the slicing segment detachably coupled to the wall of the retrieval bag, and the pulling segment free of the wall of the retrieval bag and extending out of the opening of the retrieval bag. The exactly one electrosurgical slicing wire is single-stranded or multi-stranded, as is known in the general wire art.

For some applications, the slicing segment of the electrosurgical slicing wire is detachably embedded in the wall of the retrieval bag. For some of these applications:
the slicing segment of the electrosurgical slicing wire is detachably embedded in the wall of the retrieval bag, such that the wall electrically insulates the slicing segment of the electrosurgical slicing wire, and
the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment from the wall of the retrieval bag, electrically exposes the successive longitudinal portions as they are detached from the wall of the retrieval bag, and slices the resected tissue specimen within the retrieval bag.

For some applications, the wall of the retrieval bag, in addition to or instead of electrically insulating the slicing segment of the electrosurgical slicing wire when detachable embedded in the wall, fixates the slicing segment to the wall at predetermined locations on the wall.

For some applications, the tissue removal apparatus is configured such that the pulling of the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, slices the resected tissue specimen within the retrieval bag:
into ten or fewer pieces of tissue, such as five or fewer pieces, e.g., three or fewer pieces, such as a single piece of tissue; and/or
into one or more pieces of tissue, at least one of which is long, e.g., has a length greater than a multiple of a greatest thickness of the piece of tissue perpendicular to the length, the multiple equal to 10, such as equal to 20.

The shape of the tissue may affect the number of pieces. Slicing the resected tissue specimen into a relatively small number of pieces, ideally a single long piece (i.e., a long strip) when possible, or a single long piece and a small number of smaller pieces, reduces the number of pieces that must be separately removed from the retrieval bag during the tissue removal procedure. This may substantially shorten the length of the tissue removal procedure. For example, tissue extraction from the retrieval bag, after completion of the tissue slicing, may take less than 10 minutes, typically less than 2 minutes.

For some applications, when the retrieval bag is in an expanded configuration, the retrieval bag defines a central longitudinal axis, and the slicing segment of the electrosurgical slicing wire winds around the central longitudinal axis at varying locations along the central longitudinal axis. For some of these applications, the slicing segment of the electrosurgical slicing wire is shaped as a generally three-dimensional spiral when the retrieval bag is in the expanded configuration.

For some applications, the slicing segment of the electrosurgical slicing wire is electrically insulated by an electrically-insulating strip. The electrically-insulating strip is permanently coupled to the inner surface of the wall of the retrieval bag, so as to detachably couple the slicing segment to the wall of the retrieval bag. Typically, the electrically-insulating strip comprises a polymer film. The tissue removal apparatus is configured such that the pulling of the pulling segment of the electrosurgical slicing wire (a) detaches the successive longitudinal portions of the slicing segment from the wall of the retrieval bag by tearing the electrically-insulating strip, (b) electrically exposes the successive longitudinal portions as they are detached from the wall, and (c) slices the resected tissue specimen within the retrieval bag. For some applications, the electrically-insulating strip is configured to provide tear propagation along the strip as the successive longitudinal portions of the slicing segment are detached from the wall of the retrieval bag.

For some applications, the tissue removal apparatus further comprises a buffer layer, which is coupled to the inner surface of the wall of the retrieval bag and the electrically-insulating strip, so as to permanently couple the electrically-insulating strip to the inner surface of the wall of the retrieval bag via the buffer layer. The buffer layer may comprise a thermally insulating material.

For some applications, the wall of the retrieval bag comprises an inner layer and an outer layer, which define therebetween one or more inflatable chambers. Typically, the inflatable chambers are arranged entirely around the retrieval bag. Upon inflation, the one or more inflatable chambers may help push surrounding organs and tissue farther away from the retrieval bag and the slicing segment of the electrosurgical slicing wire, thereby reducing the risk of damage to the surrounding anatomy. In addition, if the slicing segment should inadvertently tear the inner layer, the one or more inflatable chambers and/or the outer layer may prevent contact between the slicing segment and the surrounding anatomy.

For some applications, when the retrieval bag is in the expanded configuration, the electrically-insulating strip and the slicing segment of the electrosurgical slicing wire wind around the central longitudinal axis at varying locations along the central longitudinal axis. For some of these applications, when the retrieval bag is in the expanded configuration, the one or more inflatable chambers wind around the central longitudinal axis at varying locations along the central longitudinal axis, and at least a portion of the electrically-insulating strip is permanently coupled to the inner surface of the wall of the retrieval bag along the one or more inflatable chambers.

For some applications, the tissue removal apparatus further comprises an annular inflatable positioning chamber, which is fixed to and encircles an axial portion of an external surface of the retrieval bag, the axial portion axially between the opening of the retrieval bag and the base of the retrieval bag, spaced away from the opening and from the base. The annular inflatable positioning chamber typically sets the location of the retrieval bag with respect to a laparoscopic port (an opening in the abdominal wall), by allowing only a proximal portion of the retrieval bag proximal to annular inflatable positioning chamber to pass through the laparoscopic port as the retrieval bag is pulled out of the body through the laparoscopic port.

For some applications, the tissue removal apparatus further comprises a drainage chamber disposed distal to a distal base portion of the retrieval bag. A wall of the distal base portion of the retrieval bag is shaped so as to define one or more drainage openings between the interior of the retrieval bag and the drainage chamber. Providing the drainage chamber allows blood and other liquids to drain from the interior of the retrieval bag during a procedure. Excessive liquid within the interior of the retrieval bag may make the electrical cutting process more difficult.

The following Inventive Concepts are therefore provided in accordance with respective applications of the present invention.
Inventive Concept 1. A tissue removal apparatus for removing a resected tissue specimen from a body of a patient, the tissue removal apparatus for use with an RF electrosurgical generator, the tissue removal apparatus comprising:
   a retrieval bag having an opening for receiving the resected tissue specimen within the patient's body, wherein the retrieval bag is flexible and is configured to assume compressed and expanded configurations;
   an electrosurgical slicing wire that includes a slicing segment detachably coupled to a wall of the retrieval bag, and a pulling segment free of the wall of the retrieval bag and extending out of the opening of the retrieval bag; and
   a return electrode,
   wherein when the retrieval bag is in the expanded configuration:
      the retrieval bag defines a central longitudinal axis, and
      the slicing segment of the electrosurgical slicing wire winds around the central longitudinal axis at varying locations along the central longitudinal axis, and
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.
Inventive Concept 2. The apparatus according to Inventive Concept 1, wherein the tissue removal apparatus comprises exactly one electrosurgical slicing wire, which is single-stranded or multi-stranded.
Inventive Concept 3. The apparatus according to Inventive Concept 1, wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment of the electrosurgical slicing wire from the wall of the retrieval bag and slices the resected tissue specimen within the retrieval bag into ten or fewer pieces of tissue.
Inventive Concept 4. The apparatus according to Inventive Concept 3, wherein the tissue removal apparatus is configured to slice the resected tissue specimen into five or fewer pieces of tissue.
Inventive Concept 5. The apparatus according to Inventive Concept 4, wherein the tissue removal apparatus is configured to slice the resected tissue specimen into three or fewer pieces of tissue.
Inventive Concept 6. The apparatus according to Inventive Concept 5, wherein the tissue removal apparatus is configured to slice the resected tissue specimen into a single piece of tissue.
Inventive Concept 7. A tissue removal apparatus for removing a resected tissue specimen from a body of a patient, the tissue removal apparatus for use with an RF electrosurgical generator, the tissue removal apparatus comprising:
   a retrieval bag having an opening for receiving the resected tissue specimen within the patient's body;
   exactly one electrosurgical slicing wire that includes a slicing segment detachably coupled to a wall of the retrieval bag, and a pulling segment free of the wall of the retrieval bag and extending out of the opening of the retrieval bag, wherein the wire is single-stranded or multi-stranded; and
   a return electrode,
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.
Inventive Concept 8. The apparatus according to Inventive Concept 7, wherein the wire is multi-stranded and braided.
Inventive Concept 9. The apparatus according to Inventive Concept 7, wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment of the electrosurgical slicing wire from the wall of the retrieval bag and slices the resected tissue specimen within the retrieval bag into ten or fewer pieces of tissue.
Inventive Concept 10. The apparatus according to Inventive Concept 9, wherein the tissue removal apparatus is configured to slice the resected tissue specimen into five or fewer pieces of tissue.
Inventive Concept 11. The apparatus according to Inventive Concept 10, wherein the tissue removal apparatus is configured to slice the resected tissue specimen into three or fewer pieces of tissue.
Inventive Concept 12. The apparatus according to Inventive Concept 11, wherein the tissue removal apparatus is configured to slice the resected tissue specimen into a single piece of tissue.
Inventive Concept 13. A tissue removal apparatus for removing a resected tissue specimen from a body of a patient, the tissue removal apparatus for use with an RF electrosurgical generator, the tissue removal apparatus comprising:
   a retrieval bag having an opening for receiving the resected tissue specimen within the patient's body;
   an electrosurgical slicing wire that includes an electrically-insulated slicing segment detachably coupled to a wall of the retrieval bag, and a pulling segment free of the wall of the retrieval bag and extending out of the opening of the retrieval bag; and
   a return electrode,
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment from the wall of the retrieval bag, electrically exposes the successive longitudinal portions as they are detached from the wall, and slices the resected tissue specimen within the retrieval bag.
Inventive Concept 14. The apparatus according to Inventive Concept 13, wherein the tissue removal apparatus comprises exactly one electrosurgical slicing wire, which is single-stranded or multi-stranded.
Inventive Concept 15. The apparatus according to Inventive Concept 13, wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment of the electrosurgical slicing wire from the wall of the retrieval bag and slices the resected tissue specimen within the retrieval bag into ten or fewer pieces of tissue.
Inventive Concept 16. The apparatus according to Inventive Concept 15, wherein the tissue removal apparatus is configured to slice the resected tissue specimen into five or fewer pieces of tissue.
Inventive Concept 17. The apparatus according to Inventive Concept 16, wherein the tissue removal apparatus is configured to slice the resected tissue specimen into three or fewer pieces of tissue.
Inventive Concept 18. The apparatus according to Inventive Concept 17, wherein the tissue removal apparatus is configured to slice the resected tissue specimen into a single piece of tissue.
Inventive Concept 19. The apparatus according to any one of Inventive Concepts 13-18, wherein the slicing segment of the electrosurgical slicing wire is detachably embedded in the wall of the retrieval bag, such that the wall electrically insulates the slicing segment of the electrosurgical slicing wire.
Inventive Concept 20. A tissue removal apparatus for removing a resected tissue specimen from a body of a patient, the tissue removal apparatus for use with an RF electrosurgical generator, the tissue removal apparatus comprising:
   a retrieval bag having an opening for receiving the resected tissue specimen within the patient's body;
   an electrosurgical slicing wire that includes a slicing segment detachably coupled to a wall of the retrieval bag, and a pulling segment free of the wall of the retrieval bag and extending out of the opening of the retrieval bag; and
   a return electrode,
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag into ten or fewer pieces of tissue.
Inventive Concept 21. The apparatus according to Inventive Concept 20, wherein the tissue removal apparatus is configured to slice the resected tissue specimen into five or fewer pieces of tissue.
Inventive Concept 22. The apparatus according to Inventive Concept 21, wherein the tissue removal apparatus is configured to slice the resected tissue specimen into three or fewer pieces of tissue.
Inventive Concept 23. The apparatus according to Inventive Concept 22, wherein the tissue removal apparatus is configured to slice the resected tissue specimen into a single piece of tissue.
Inventive Concept 24. The apparatus according to Inventive Concept 20, wherein the tissue removal apparatus is configured to slice the resected tissue specimen such that at least one of the pieces of tissue has a length greater than a multiple of a greatest thickness of the piece of tissue perpendicular to the length, the multiple equal to 10.
Inventive Concept 25. The apparatus according to Inventive Concept 24, wherein the multiple is equal to 20.
Inventive Concept 26. A tissue removal apparatus for removing a resected tissue specimen from a body of a patient, the tissue removal apparatus for use with an RF electrosurgical generator, the tissue removal apparatus comprising:
   a retrieval bag having an opening for receiving the resected tissue specimen within the patient's body;
   an electrosurgical slicing wire that includes a slicing segment detachably coupled to a wall of the retrieval bag, and a pulling segment free of the wall of the retrieval bag and extending out of the opening of the retrieval bag; and
   a return electrode,
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag into one or more pieces of tissue, at least one of which has a length greater than a multiple of a greatest thickness of the piece of tissue perpendicular to the length, the multiple equal to 10.
Inventive Concept 27. The apparatus according to Inventive Concept 26, wherein the multiple is equal to 20.
Inventive Concept 28. The apparatus according to any one of Inventive Concepts 20 and 26, wherein the tissue removal apparatus comprises exactly one electrosurgical slicing wire.
Inventive Concept 29. The apparatus according to any one of Inventive Concepts 1-28,
   further comprising an electrically-insulating strip comprising a polymer film,
   wherein the strip is permanently coupled to an inner surface of the wall of the retrieval bag, so as to detachably couple the slicing segment to the wall of the retrieval bag, and
   wherein the tissue removal apparatus is configured such that the pulling of the pulling segment of the electrosurgical slicing wire (a) detaches the successive longitudinal portions of the slicing segment from the wall of the retrieval bag by tearing the strip, (b) electrically exposes the successive longitudinal portions as they are detached from the wall, and (c) slices the resected tissue specimen within the retrieval bag.
Inventive Concept 30. The apparatus according to Inventive Concept 29, wherein the electrically-insulating strip is configured to provide tear propagation along the electrically-insulating strip as the successive longitudinal portions of the slicing segment from the wall of the retrieval bag.
Inventive Concept 31. The apparatus according to Inventive Concept 29,
   wherein the retrieval bag is flexible and is configured to assume compressed and expanded configurations, and
   wherein when the retrieval bag is in the expanded configuration:
      the retrieval bag defines a central longitudinal axis, and
      the strip and the slicing segment of the electrosurgical slicing wire wind around the central longitudinal axis at varying locations along the central longitudinal axis.
Inventive Concept 32. The apparatus according to Inventive Concept 31,
   wherein the wall of the retrieval bag comprises an inner layer and an outer layer, which define therebetween one or more inflatable chambers, and
   wherein when the retrieval bag is in the expanded configuration:
      the one or more inflatable chambers wind around the central longitudinal axis at varying locations along the central longitudinal axis, and
      at least a portion of the strip is permanently coupled to the inner surface of the wall of the retrieval bag along the one or more inflatable chambers that wind around the central longitudinal axis, so as to detachably couple the slicing segment to the wall of the retrieval bag along the one or more inflatable chambers that wind around the central longitudinal axis.
Inventive Concept 33. The apparatus according to Inventive Concept 29, wherein the electrically-insulating strip comprises an adhesive coated onto the polymer film.
Inventive Concept 34. The apparatus according to Inventive Concept 33, wherein the strip is permanently coupled to the inner surface of the wall of the retrieval bag by the adhesive.
Inventive Concept 35. The apparatus according to Inventive Concept 34, wherein the slicing segment is sandwiched between the strip and the inner surface of the wall of the retrieval bag.
Inventive Concept 36. The apparatus according to Inventive Concept 33,
   wherein the adhesive is disposed on only a first surface of the polymer film, and
   wherein the polymer film is folded and wrapped around over the slicing segment such that two portions of the first surface are adhered to each other.
Inventive Concept 37. The apparatus according to Inventive Concept 36, wherein a second surface of the polymer film, opposite the first surface, is permanently coupled to the inner surface of the wall of the retrieval bag, so as to detachably couple the slicing segment to the wall of the retrieval bag.
Inventive Concept 38. The apparatus according to Inventive Concept 37, wherein the second surface of the polymer film is permanently coupled to the inner surface of the wall of the retrieval bag by polymer welding.
Inventive Concept 39. The apparatus according to Inventive Concept 37, wherein the adhesive is a first adhesive, wherein the apparatus further comprises a second adhesive, and wherein the second surface of the polymer film is permanently coupled to the inner surface of the wall of the retrieval bag by the second adhesive.
Inventive Concept 40. The apparatus according to Inventive Concept 29, wherein the polymer film is folded and wrapped around over the slicing segment such that two portions of a first surface of the polymer film are coupled to each other.
Inventive Concept 41. The apparatus according to Inventive Concept 40, wherein a second surface of the polymer film, opposite the first surface, is permanently coupled to the inner surface of the wall of the retrieval bag, so as to detachably couple the slicing segment to the wall of the retrieval bag.
Inventive Concept 42. The apparatus according to Inventive Concept 41, wherein the second surface of the polymer film is permanently coupled to the inner surface of the wall of the retrieval bag by polymer welding.
Inventive Concept 43. The apparatus according to Inventive Concept 29, further comprising a buffer layer, which is coupled to the inner surface of the wall of the retrieval bag and the strip, so as to permanently couple the strip to the inner surface of the wall of the retrieval bag via the buffer layer.
Inventive Concept 44. The apparatus according to Inventive Concept 43, wherein the buffer layer comprises a thermally insulating material.
Inventive Concept 45. The apparatus according to Inventive Concept 29, wherein a length of the strip is at least 100 cm, and wherein the slicing segment runs along the length of the strip.
Inventive Concept 46. The apparatus according to Inventive Concept 29, wherein a length of the strip is greater than or equal to a length of the slicing segment, and wherein the slicing segment runs along the length of the strip.
Inventive Concept 47. The apparatus according to Inventive Concept 29, wherein the polymer film comprises polypropylene or cellulose acetate.
Inventive Concept 48. The apparatus according to Inventive Concept 29, wherein the polymer film comprises a polymer selected from the group of polymers consisting of: biaxially oriented polypropylene (BOPP), a polyimide, polypropylene carbonate (PPC), and biaxially oriented polyethylene terephthalate (BoPET).
Inventive Concept 49. The apparatus according to any one of Inventive Concepts 1-28, wherein the wall of the retrieval bag comprises an inner layer and an outer layer, which define therebetween one or more inflatable chambers.
Inventive Concept 50. The apparatus according to Inventive Concept 49, wherein the inner layer and the outer layer are welded together at a plurality of welding locations so as to define the one or more inflatable chambers therebetween.
Inventive Concept 51. The apparatus according to Inventive Concept 49,
   wherein the retrieval bag is flexible and is configured to assume compressed and expanded configurations, and
   wherein when the retrieval bag is in the expanded configuration:
      the retrieval bag defines a central longitudinal axis, and
      the one or more inflatable chambers wind around the central longitudinal axis at varying locations along the central longitudinal axis.
Inventive Concept 52. The apparatus according to Inventive Concept 49,
   wherein the retrieval bag is shaped so as to define a base portion opposite the opening,
   wherein the wall includes (a) a first portion that defines the base portion, and (b) a second portion that is closer to the opening than the first portion is to the opening,
   wherein the first portion of the wall is defined by three or more triangles, which:
      have respective first base edges that are integral with the second portion of the wall,
      have respective second and third curved edges that meet at respective apexes, and
      comprise respective portions of the inner layer and the outer layer, which define therebetween respective portions of the one or more inflatable chambers, and
   wherein the second curved edges are fixed to the third curved edges of adjacent respective ones of the triangles, such that the triangles together define the base portion and the apexes together define a base of the base portion.
Inventive Concept 53. The apparatus according to Inventive Concept 52,
   wherein the inner layer and the outer layer are welded together at a plurality of welding locations so as to define the one or more inflatable chambers therebetween, and
   wherein the second curved edges are welded to the third curved edges of the adjacent respective ones of the triangles.
Inventive Concept 54. The apparatus according to any one of Inventive Concepts 1-28, wherein the slicing segment of the electrosurgical slicing wire is detachably embedded in the wall of the retrieval bag.
Inventive Concept 55. The apparatus according to Inventive Concept 54,
   wherein the slicing segment of the electrosurgical slicing wire is detachably embedded in the wall of the retrieval bag, such that the wall electrically insulates the slicing segment of the electrosurgical slicing wire, and
   wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment from the wall of the retrieval bag, electrically exposes the successive longitudinal portions as they are detached from the wall, and slices the resected tissue specimen within the retrieval bag.
Inventive Concept 56. The apparatus according to any one of Inventive Concepts 19 and 55,
   wherein the wall of the retrieval bag comprises an inner electrical insulation layer and an outer electrical insulation layer, disposed outward of the inner electrical insulation layer with respect to an interior of the retrieval bag,
   wherein the slicing segment of the electrosurgical slicing wire is detachably embedded in the wall of the retrieval bag between the inner electrical insulation layer and the outer electrical insulation layer, and
   wherein the tissue removal apparatus is configured such that the pulling of the pulling segment of the electrosurgical slicing wire, during the application of the RF electrosurgical power between the electrosurgical slicing wire and the return electrode, pulls the successive longitudinal portions of the slicing segment of the electrosurgical slicing wire through the inner electrical insulation layer and detaches the successive longitudinal portions from the outer electrical insulation layer, thereby electrically exposing the successive longitudinal portions.
Inventive Concept 57. The apparatus according to Inventive Concept 56, wherein a thickness of the outer electrical insulation layer is greater than a thickness of the inner electrical insulation layer.
Inventive Concept 58. The apparatus according to Inventive Concept 57, wherein the thickness of the outer electrical insulation layer equals at least 110% of the thickness of the inner electrical insulation layer.
Inventive Concept 59. The apparatus according to Inventive Concept 56, wherein the inner electrical insulation layer and the outer electrical insulation layer are adhered to each other.
Inventive Concept 60. The apparatus according to any one of Inventive Concepts 19 and 55,
   wherein the wall of the retrieval bag comprises:
   one or more electrical insulation layers that electrically insulate the slicing segment of the electrosurgical slicing wire; and
   an electrically-conductive safety layer, disposed outward of the one or more electrical insulation layers, and
   wherein the retrieval bag further comprises an electrical lead that is electrically coupled to the electrically-conductive safety layer and terminates outside the retrieval bag.
Inventive Concept 61. The apparatus according to Inventive Concept 60, wherein the electrically-conductive safety layer comprises an at least partially electrically conductive polymer.
Inventive Concept 62. A tissue removal system comprising the apparatus according to
Inventive Concept 60, the tissue removal system further comprising a voltage monitoring circuit,
   wherein the electrical lead is couplable to the voltage monitoring circuit, and
   wherein the tissue removal system is configured to cease the application of the RF electrosurgical power between the electrosurgical slicing wire and the return electrode upon sensing, by the voltage monitoring circuit, that a voltage in the electrically-conductive safety layer exceeds a threshold level indicative of electrical leakage through the one or more electrical insulation layers of the wall of the retrieval bag to the electrically-conductive safety layer.
Inventive Concept 63. The apparatus according to any one of Inventive Concepts 19 and 55, wherein the slicing segment of the electrosurgical slicing wire is not surrounded by electrical insulation distinct from the wall of the retrieval bag.
Inventive Concept 64. The apparatus according to any one of Inventive Concepts 19 and 55, wherein the slicing segment of the electrosurgical slicing wire is detachably embedded in a single electrical insulation layer of the wall of the retrieval bag.
Inventive Concept 65. The apparatus according to any one of the preceding Inventive Concepts, wherein a length of the slicing segment is at least 100 cm.
Inventive Concept 66. The apparatus according to any one of the preceding Inventive Concepts, wherein a length of the slicing segment of the electrosurgical slicing wire equals at least 4 times a perimeter of the opening of the retrieval bag.
Inventive Concept 67. The apparatus according to any one of Inventive Concepts 7-28 and 56-66,
   wherein the retrieval bag is flexible and is configured to assume compressed and expanded configurations, and
   wherein when the retrieval bag is in the expanded configuration:
   the retrieval bag defines a central longitudinal axis, and
   the slicing segment of the electrosurgical slicing wire winds around the central longitudinal axis at varying locations along the central longitudinal axis.
Inventive Concept 68. The apparatus according to any one of Inventive Concepts 1 and 67, wherein the slicing segment of the electrosurgical slicing wire winds around the central longitudinal axis in at least 4 turns.
Inventive Concept 69. The apparatus according to any one of Inventive Concepts 1 and 67, wherein when the retrieval bag is in the expanded configuration:
   the opening is at a proximal end of the retrieval bag opposite a distal end of the retrieval bag at a base of the retrieval bag, and
   (a) a first location on the central longitudinal axis corresponding to the opening of the retrieval bag is at least 5 cm from (b) a second location on the central longitudinal axis corresponding to a proximal-most location on the wall of the retrieval bag at which the slicing segment is detachably coupled to the wall.
Inventive Concept 70. The apparatus according to any one of Inventive Concepts 1 and 67, wherein the retrieval bag is shaped so as to define a base opposite the opening, and wherein the slicing segment of the electrosurgical slicing wire winds around the central longitudinal axis in a base-to-opening direction at a nondecreasing distance from the central longitudinal axis when the retrieval bag is in the expanded configuration.
Inventive Concept 71. The apparatus according to Inventive Concept 70, wherein the slicing segment of the electrosurgical slicing wire is shaped as a generally three-dimensional spiral when the retrieval bag is in the expanded configuration.
Inventive Concept 72. The apparatus according to any one of Inventive Concepts 7-28 and 56-66,
   wherein the retrieval bag is flexible and is configured to assume compressed and expanded configurations, and to define a central longitudinal axis when the retrieval bag is in the expanded configuration, and
   wherein the slicing segment of the electrosurgical slicing wire is detachably coupled to the wall of the retrieval bag such that the slicing segment travels around the central longitudinal axis in at least four turns in alternating clockwise and counterclockwise directions in axially sequential turns when the retrieval bag is in the expanded configuration.
Inventive Concept 73. The apparatus according to any one of the preceding Inventive Concepts,
   wherein the pulling segment is proximal, along the electrosurgical slicing wire, to the slicing segment of the electrosurgical slicing wire, and
   wherein the tissue removal apparatus is configured such that the pulling of the pulling segment of the electrosurgical slicing wire detaches, from the wall of the retrieval bag, the successive longitudinal portions in a proximal-to-distal direction along the slicing segment of the electrosurgical slicing wire.
Inventive Concept 74. The apparatus according to any one of the preceding Inventive Concepts, further comprising a containment bag within which the retrieval bag is disposed, so as to define one or more inflatable chambers between an inner surface of the containment bag and an outer surface of the retrieval bag.
Inventive Concept 75. The apparatus according to Inventive Concept 74, further comprising an inflatable-chambers inflation tube in fluid contact with all of the one or more inflatable chambers.
Inventive Concept 76. The apparatus according to Inventive Concept 74,
   wherein a wall of the containment bag is shaped so as to define a plurality of inflatable compartments arranged entirely around the containment bag, and
   wherein the tissue removal apparatus comprises an inflatable-compartments inflation tube in fluid contact with all of the inflatable compartments.
Inventive Concept 77. The apparatus according to Inventive Concept 76,
   wherein the retrieval bag is flexible and is configured to assume compressed and expanded configurations, and to define a central longitudinal axis when the retrieval bag is in the expanded configuration, and
   wherein (a) a location along the central longitudinal axis corresponding to a proximal edge of the plurality of inflatable compartments is at or proximal to (b) a location along the central longitudinal axis corresponding to a proximal-most location on the wall of the retrieval bag at which the slicing segment is detachably coupled to the wall of the retrieval bag, when the retrieval bag is in the expanded configuration.
Inventive Concept 78. The apparatus according to Inventive Concept 74, wherein the containment bag comprises a camera port open to one of the one or more inflatable chambers.
Inventive Concept 79. The apparatus according to any one of the preceding Inventive Concepts, wherein the return electrode is coupled to the retrieval bag.
Inventive Concept 80. The apparatus according to any one of the preceding Inventive Concepts, wherein the resected tissue specimen is a resected uterus, and wherein the retrieval bag is sized to receive, via the opening, the resected uterus within the patient's body.
Inventive Concept 81. The apparatus according to any one of the preceding Inventive Concepts, wherein the resected tissue specimen is a resected fibroid, and wherein the retrieval bag is sized to receive, via the opening, the resected fibroid within the patient's body.
Inventive Concept 82. The apparatus according to any one of the preceding Inventive Concepts, wherein the resected tissue specimen is all or a portion of an organ, and wherein the retrieval bag is sized to receive, via the opening, the resected all or a portion of the organ within the patient's body.
Inventive Concept 83. The apparatus according to Inventive Concept 82, wherein the organ is selected from the group of organs consisting of: a resected spleen, a resected kidney, and a resected lung.
Inventive Concept 84. A tissue removal system comprising the apparatus according to any one of the preceding Inventive Concepts, the tissue removal system further comprising a control handle, which is shaped so as to define an elongate insertion tip, which is shaped for insertion into the patient's body and is shaped so as to define a channel therethrough for insertion of the pulling segment of the electrosurgical slicing wire.
Inventive Concept 85. A tissue removal system comprising the apparatus according to any one of the preceding Inventive Concepts, the tissue removal system further comprising a control handle, which comprises the return electrode.
Inventive Concept 86. A tissue removal system comprising the apparatus according to any one of the preceding Inventive Concepts, the tissue removal system further comprising a control handle,
   wherein the pulling segment of the electrosurgical slicing wire is electrically and mechanically couplable to the control handle, and
   wherein the control handle comprises a motor arranged to pull the pulling segment of the electrosurgical slicing wire during the application of RF electrosurgical power between the electrosurgical slicing wire and the return electrode, when the pulling segment of the electrosurgical slicing wire is electrically and mechanically coupled to the control handle.
Inventive Concept 87. The system according to Inventive Concept 86, wherein the control handle comprises a user control, and wherein the user control activates the control handle in:
   a first step, in which the control handle activates the motor to pull the pulling segment of the electrosurgical slicing wire without applying the RF electrosurgical power to the electrosurgical slicing wire, and
   a second step, in which the control handle, in addition to activating the motor to pull the pulling segment of the electrosurgical slicing wire, applies the RF electrosurgical power to the pulling segment.
Inventive Concept 88. The system according to Inventive Concept 87, wherein the user control activates the control handle in the first step to activate the motor to pull the pulling segment of the electrosurgical slicing wire without applying the RF electrosurgical power to the electrosurgical slicing wire, until a longitudinal portion of the slicing segment of the electrosurgical slicing wire comes detached from the wall of the retrieval bag.
Inventive Concept 89. A tissue removal system comprising the apparatus according to any one of the preceding Inventive Concepts, the tissue removal system further comprising the RF electrosurgical generator.
Inventive Concept 90. A tissue removal system comprising the apparatus according to any one of the preceding Inventive Concepts, the tissue removal system further comprising a conductive gel for filling at least a portion of empty spaces between the resected tissue specimen and an inner surface of the retrieval bag when the resected tissue specimen is within the retrieval bag.
Inventive Concept 91. A tissue removal apparatus for removing a resected tissue specimen from a body of a patient, the tissue removal apparatus for use with an RF electrosurgical generator, the tissue removal apparatus comprising:
   a retrieval bag shaped so as to define an opening for receiving the resected tissue specimen within the patient's body, and a base portion opposite the opening;
   an electrosurgical slicing wire that includes a slicing segment; and
   a return electrode,
   wherein a wall of the retrieval bag comprises an inner layer and an outer layer, which define therebetween one or more inflatable chambers,
   wherein the wall includes (a) a first portion that defines the base portion, and (b) a second portion that is closer to the opening than the first portion is to the opening,
   wherein the first portion of the wall is defined by three or more triangles, which:
      have respective first base edges that are integral with the second portion of the wall,
      have respective second and third curved edges that meet at respective apexes, and
      comprise respective portions of the inner layer and the outer layer, which define therebetween respective portions of the one or more inflatable chambers, and
   wherein the second curved edges are fixed to the third curved edges of adjacent respective ones of the triangles, such that the triangles together define the base portion and the apexes together define a base of the base portion.
Inventive Concept 92. The apparatus according to Inventive Concept 91,
   wherein the inner layer and the outer layer are welded together at a plurality of welding locations so as to define the one or more inflatable chambers therebetween, and
   wherein the second curved edges are welded to the third curved edges of the adjacent respective ones of the triangles.
Inventive Concept 93. The apparatus according to Inventive Concept 91,
   wherein the retrieval bag is flexible and is configured to assume compressed and expanded configurations, and
   wherein when the retrieval bag is in the expanded configuration:
      the retrieval bag defines a central longitudinal axis, and
      the one or more inflatable chambers wind around the central longitudinal axis at varying locations along the central longitudinal axis.
Inventive Concept 94. The apparatus according to Inventive Concept 91,
   wherein the slicing segment of the electrosurgical slicing wire is detachably coupled to a wall of the retrieval bag,
   wherein the electrosurgical slicing wire further includes a pulling segment free of the wall of the retrieval bag and extending out of the opening of the retrieval bag, and
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.
Inventive Concept 95. A tissue removal apparatus for removing a resected tissue specimen from a body of a patient, the tissue removal apparatus comprising:
   a retrieval bag shaped so as to define an opening for receiving the resected tissue specimen within the patient's body, and a base opposite the opening, wherein the retrieval bag is flexible and is configured to assume compressed and expanded configurations, and to define a central longitudinal axis when the retrieval bag is in the expanded configuration; and
   an annular inflatable positioning chamber, which is fixed to and encircles an axial portion of an external surface of the retrieval bag, the axial portion axially between the opening and the base, spaced away from the opening and from the base.
Inventive Concept 96. The apparatus according to Inventive Concept 95, wherein the annular inflatable positioning chamber is in fluid communication with an interior of the retrieval bag.
Inventive Concept 97. The apparatus according to Inventive Concept 95, wherein, when the retrieval bag is in the expanded configuration and the annular inflatable positioning chamber is fully inflated, a greatest external diameter of the annular inflatable positioning chamber equals 100%-200% of a greatest external diameter of the retrieval bag.
Inventive Concept 98. The apparatus according to Inventive Concept 95, wherein, when the retrieval bag is in the expanded configuration, the axial portion of the external surface of the retrieval bag is generally conical.
Inventive Concept 99. The apparatus according to Inventive Concept 98, wherein, when the retrieval bag is in the expanded configuration and the annular inflatable positioning chamber is fully inflated, an external surface of the annular inflatable positioning chamber is generally cylindrical.
Inventive Concept 100. The apparatus according to Inventive Concept 95, wherein the external surface of the axial portion of the retrieval bag serves as an internal wall of the annular inflatable positioning chamber.
Inventive Concept 101. The apparatus according to Inventive Concept 95, wherein the wall of the retrieval bag comprises an inner layer and an outer layer, which define therebetween one or more inflatable chambers.
Inventive Concept 102. The apparatus according to Inventive Concept 95, wherein the tissue removal apparatus further comprises (a) an electrosurgical slicing wire that includes a slicing segment and (b) a return electrode.
Inventive Concept 103. The apparatus according to Inventive Concept 102,
   wherein the slicing segment of the electrosurgical slicing wire is detachably coupled to a wall of the retrieval bag,
   wherein the electrosurgical slicing wire further includes a pulling segment free of the wall of the retrieval bag and extending out of the opening of the retrieval bag, and
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.
Inventive Concept 104. A tissue removal apparatus for removing a resected tissue specimen from a body of a patient, the tissue removal apparatus comprising:
   a retrieval bag shaped so as to define a proximal opening for receiving the resected tissue specimen within the patient's body, and a distal base portion opposite the opening; and
   a drainage chamber disposed distal to the distal base portion, wherein a wall of the distal base portion of the retrieval bag is shaped so as to define one or more drainage openings between an interior of the retrieval bag and the drainage chamber.
Inventive Concept 105. The apparatus according to Inventive Concept 104, further comprising a tube coupled in fluid communication with the drainage chamber.
Inventive Concept 106. The apparatus according to Inventive Concept 104, wherein an external surface of at least a portion of the wall of the distal base portion of the retrieval bag serves as an internal wall of the drainage chamber.
Inventive Concept 107. The apparatus according to Inventive Concept 104,
   wherein the retrieval bag is flexible and is configured to assume compressed and expanded configurations, and
   wherein, when the retrieval bag is in the expanded configuration, a volume of the drainage chamber equals 0.5%-5% of a volume of the retrieval bag.
Inventive Concept 108. The apparatus according to Inventive Concept 104, wherein the tissue removal apparatus further comprises (a) an electrosurgical slicing wire that includes a slicing segment and (b) a return electrode.
Inventive Concept 109. The apparatus according to Inventive Concept 108,
   wherein the slicing segment of the electrosurgical slicing wire is detachably coupled to a wall of the retrieval bag,
   wherein the electrosurgical slicing wire further includes a pulling segment free of the wall of the retrieval bag and extending out of the opening of the retrieval bag, and
   wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.
Inventive Concept 110. A tissue removal apparatus for removing a resected tissue specimen from a body of a patient, the tissue removal apparatus for use with an RF electrosurgical generator, the tissue removal apparatus comprising:
   a retrieval bag having an opening for receiving the resected tissue specimen within the patient's body;
   a return electrode; and
   an electrosurgical electrode, which is configured, during application of RF electrosurgical power between the electrosurgical electrode and the return electrode when the resected tissue specimen is within the retrieval bag and the return electrode is in electrical contact with the resected tissue specimen, to cut the resected tissue specimen within the retrieval bag,
   wherein the retrieval bag comprises a wall, which comprises:
      one or more electrical insulation layers, which electrically isolate at least a portion of the electrosurgical electrode from portions of the patient's body outside the retrieval bag, when the retrieval bag is disposed partially within the patient's body; and
      an electrically-conductive safety layer, disposed outward of the one or more electrical insulation layers, and
   wherein the retrieval bag further comprises an electrical lead that is electrically coupled to the electrically-conductive safety layer and terminates outside the retrieval bag.
Inventive Concept 111. The apparatus according to Inventive Concept 110, wherein the electrically-conductive safety layer comprises an at least partially electrically conductive polymer.
Inventive Concept 112. A tissue removal system comprising the apparatus according to Inventive Concept 110, the tissue removal system further comprising a voltage monitoring circuit,
   wherein the electrical lead is couplable to the voltage monitoring circuit, and
   wherein the tissue removal system is configured to cease the application of the RF electrosurgical power between the electrosurgical electrode and the return electrode upon sensing, by the voltage monitoring circuit, that a voltage in the electrically-conductive safety layer exceeds a threshold level indicative of electrical penetration through the one or more electrical insulation layers of the wall of the retrieval bag to the electrically-conductive safety layer.
Inventive Concept 113. The apparatus according to Inventive Concept 110, wherein the electrosurgical electrode comprises an electrosurgical slicing wire.
Inventive Concept 114. A tissue removal apparatus for removing a resected tissue specimen from a body of a patient, the tissue removal apparatus comprising:
   a retrieval bag having an opening for receiving the resected tissue specimen within the patient's body;
   one or more tissue cutters detachably coupled to a wall of the retrieval bag, which are configured to cut the resected tissue specimen when the resected tissue specimen is within the retrieval bag; and
   a containment bag within which the retrieval bag is disposed, so as to define one or more inflatable chambers between an inner surface of the containment bag and an outer surface of the retrieval bag.
Inventive Concept 115. The apparatus according to Inventive Concept 114, wherein the one or more tissue cutters comprise one or more cutting wires.
Inventive Concept 116. The apparatus according to Inventive Concept 114, further comprising an inflatable-chambers inflation tube in fluid contact with all of the one or more inflatable chambers.
Inventive Concept 117. The apparatus according to Inventive Concept 114,
   wherein a wall of the containment bag is shaped so as to define a plurality of inflatable compartments arranged entirely around the containment bag, and
   wherein the tissue removal apparatus comprises an inflatable-compartments inflation tube in fluid contact with all of the inflatable compartments.
Inventive Concept 118. The apparatus according to Inventive Concept 117,
   wherein the retrieval bag is flexible and is configured to assume compressed and expanded configurations, and to define a central longitudinal axis when the retrieval bag is in the expanded configuration, and
   wherein (a) a location along the central longitudinal axis corresponding to a proximal edge of the plurality of inflatable compartments is at or proximal to (b) a location along the central longitudinal axis corresponding to a proximal-most point at which the one or more tissue cutters are detachably coupled to the wall of the retrieval bag, when the retrieval bag is in the expanded configuration.
Inventive Concept 119. The apparatus according to Inventive Concept 114, wherein the containment bag comprises a camera port open to one of the one or more inflatable chambers.
Inventive Concept 120. The apparatus according to any one of Inventive Concepts 114-119, for use with a generator, the tissue removal apparatus further comprising one or more electrical leads for coupling the one or more tissue cutters to the generator.
Inventive Concept 121. A tissue removal system for removing a resected tissue specimen from a body of a patient, the tissue removal system comprising:
   a tissue removal apparatus comprising:
      a retrieval bag having an opening for receiving the resected tissue specimen within the patient's body; and
      one or more tissue cutters detachably coupled to a wall of the retrieval bag, which are configured to cut the resected tissue specimen when the resected tissue specimen is within the retrieval bag; and
   a conductive gel for filling at least a portion of empty spaces between the resected tissue specimen and an inner surface of the retrieval bag when the resected tissue specimen is within the retrieval bag.
Inventive Concept 122. The system according to Inventive Concept 121, wherein the one or more tissue cutters comprise one or more cutting wires.
Inventive Concept 123. The system according to Inventive Concept 121, for use with a generator, the tissue removal system further comprising one or more electrical leads for coupling the one or more tissue cutters to the generator.
Inventive Concept 124. A method for removing a resected tissue specimen from a body of a patient, the method comprising:
   inserting, into the patient's body, a flexible retrieval bag of a tissue removal apparatus while the retrieval bag is in a compressed configuration, wherein the tissue removal apparatus further includes (a) an electrosurgical slicing wire that includes a slicing segment detachably coupled to a wall of the retrieval bag, and a pulling segment free of the wall of the retrieval bag and extending out of an opening of the retrieval bag, and (b) a return electrode, wherein the retrieval bag is configured to assume an expanded configuration in which the retrieval bag defines a central longitudinal axis, and the slicing segment of the electrosurgical slicing wire winds around the central longitudinal axis at varying locations along the central longitudinal axis;
   inserting the resected tissue specimen into the retrieval bag via the opening, while the retrieval bag is in the patient's body;
   pulling the pulling segment of the electrosurgical slicing wire, during application of RF electrosurgical power between the electrosurgical slicing wire and the return electrode when the resected tissue specimen is within the retrieval bag in the expanded configuration and the return electrode is in electrical contact with the resected tissue specimen, thereby detaching successive longitudinal portions of the slicing segment of the electrosurgical slicing wire from the wall of the retrieval bag and slicing the resected tissue specimen within the retrieval bag;
   while the retrieval bag is partially within the patient's body such that the opening of the retrieval bag is outside the patient's body, removing the sliced resected tissue specimen from the retrieval bag and the patient's body; and
   thereafter, removing the retrieval bag from the patient's body.
Inventive Concept 125. The method according to Inventive Concept 124, wherein the tissue removal apparatus includes exactly one electrosurgical slicing wire, which is single-stranded or multi-stranded.
Inventive Concept 126. The method according to Inventive Concept 124, wherein pulling the pulling segment of the electrosurgical slicing wire comprises pulling the pulling segment of the electrosurgical slicing wire, during the application of RF electrosurgical power between the electrosurgical slicing wire and the return electrode, thereby detaching the successive longitudinal portions of the slicing segment of the electrosurgical slicing wire from the wall of the retrieval bag and slicing the resected tissue specimen within the retrieval bag into ten or fewer pieces of tissue.
Inventive Concept 127. The method according to Inventive Concept 126, wherein pulling the pulling segment of the electrosurgical slicing wire slices the resected tissue specimen within the retrieval bag into five or fewer pieces of tissue.
Inventive Concept 128. The method according to Inventive Concept 127, wherein pulling the pulling segment of the electrosurgical slicing wire slices the resected tissue specimen into three or fewer pieces of tissue.
Inventive Concept 129. The method according to Inventive Concept 128, wherein pulling the pulling segment of the electrosurgical slicing wire slices the resected tissue specimen into a single piece of tissue.
Inventive Concept 130. A method for removing a resected tissue specimen from a body of a patient, the method comprising:
   inserting, into the patient's body, a retrieval bag of a tissue removal apparatus, wherein the tissue removal apparatus further includes (a) exactly one electrosurgical slicing wire that includes a slicing segment detachably coupled to a wall of the retrieval bag, and a pulling segment free of the wall of the retrieval bag and extending out of an opening of the retrieval bag, wherein the wire is single-stranded or multi-stranded, and (b) a return electrode;
   inserting the resected tissue specimen into the retrieval bag via the opening, while the retrieval bag is in the patient's body;
   pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, thereby detaching successive longitudinal portions of the slicing segment of the electrosurgical slicing wire from the wall of the retrieval bag and slicing the resected tissue specimen within the retrieval bag;
   while the retrieval bag is partially within the patient's body such that the opening of the retrieval bag is outside the patient's body, removing the sliced resected tissue specimen from the retrieval bag and the patient's body; and
   thereafter, removing the retrieval bag from the patient's body.
Inventive Concept 131. The method according to Inventive Concept 130, wherein the wire is multi-stranded and braided.
Inventive Concept 132. The method according to Inventive Concept 130, wherein pulling the pulling segment of the electrosurgical slicing wire comprises pulling the pulling segment of the electrosurgical slicing wire, during the application of RF electrosurgical power between the electrosurgical slicing wire and the return electrode, thereby detaching the successive longitudinal portions of the slicing segment of the electrosurgical slicing wire from the wall of the retrieval bag and slicing the resected tissue specimen within the retrieval bag into ten or fewer pieces of tissue.
Inventive Concept 133. The method according to Inventive Concept 132, wherein pulling the pulling segment of the electrosurgical slicing wire slices the resected tissue specimen within the retrieval bag into five or fewer pieces of tissue.
Inventive Concept 134. The method according to Inventive Concept 133, wherein pulling the pulling segment of the electrosurgical slicing wire slices the resected tissue specimen into three or fewer pieces of tissue.
Inventive Concept 135. The method according to Inventive Concept 134, pulling the pulling segment of the electrosurgical slicing wire slices the resected tissue specimen into a single piece of tissue.
Inventive Concept 136. A method for removing a resected tissue specimen from a body of a patient, the method comprising:
   inserting, into the patient's body, a retrieval bag of a tissue removal apparatus, wherein the tissue removal apparatus further includes (a) an electrosurgical slicing wire that includes an electrically-insulated slicing segment detachably coupled to a wall of the retrieval bag, and a pulling segment free of the wall of the retrieval bag and extending out of an opening of the retrieval bag, wherein the wire is single-stranded or multi-stranded, and (b) a return electrode;
   inserting the resected tissue specimen into the retrieval bag via the opening, while the retrieval bag is in the patient's body;
   pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, thereby detaching successive longitudinal portions of the slicing segment of the electrosurgical slicing wire from the wall of the retrieval bag, electrically exposing the successive longitudinal portions as they are detached from the wall, and slicing the resected tissue specimen within the retrieval bag;
   while the retrieval bag is partially within the patient's body such that the opening of the retrieval bag is outside the patient's body, removing the sliced resected tissue specimen from the retrieval bag and the patient's body; and
   thereafter, removing the retrieval bag from the patient's body.
Inventive Concept 137. The method according to Inventive Concept 136, wherein the tissue removal apparatus includes exactly one electrosurgical slicing wire, which is single-stranded or multi-stranded.
Inventive Concept 138. The method according to Inventive Concept 136, wherein pulling the pulling segment of the electrosurgical slicing wire comprises pulling the pulling segment of the electrosurgical slicing wire, during the application of RF electrosurgical power between the electrosurgical slicing wire and the return electrode, thereby detaching the successive longitudinal portions of the slicing segment of the electrosurgical slicing wire from the wall of the retrieval bag and slicing the resected tissue specimen within the retrieval bag into ten or fewer pieces of tissue.
Inventive Concept 139. The method according to Inventive Concept 138, wherein pulling the pulling segment of the electrosurgical slicing wire slices the resected tissue specimen within the retrieval bag into five or fewer pieces of tissue.
Inventive Concept 140. The method according to Inventive Concept 139, wherein pulling the pulling segment of the electrosurgical slicing wire slices the resected tissue specimen within the retrieval bag into three or fewer pieces of tissue.
Inventive Concept 141. The method according to Inventive Concept 140, wherein pulling the pulling segment of the electrosurgical slicing wire slices the resected tissue specimen within the retrieval bag into a single piece of tissue.
Inventive Concept 142. The method according to any one of Inventive Concepts 136-141, wherein the slicing segment of the electrosurgical slicing wire is detachably embedded in the wall of the retrieval bag, such that the wall electrically insulates the slicing segment of the electrosurgical slicing wire.
Inventive Concept 143. A method for removing a resected tissue specimen from a body of a patient, the method comprising:
   inserting, into the patient's body, a retrieval bag of a tissue removal apparatus, wherein the tissue removal apparatus further includes (a) an electrosurgical slicing wire that includes a slicing segment detachably coupled to a wall of the retrieval bag, and a pulling segment free of the wall of the retrieval bag and extending out of an opening of the retrieval bag, and (b) a return electrode;
   inserting the resected tissue specimen into the retrieval bag via the opening, while the retrieval bag is in the patient's body;
   pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, thereby detaching successive longitudinal portions of the slicing segment of the electrosurgical slicing wire from the wall of the retrieval bag and slicing the resected tissue specimen within the retrieval bag into ten or fewer pieces of tissue;
   while the retrieval bag is partially within the patient's body such that the opening of the retrieval bag is outside the patient's body, removing the ten or fewer pieces of tissue from the retrieval bag and the patient's body; and
   thereafter, removing the retrieval bag from the patient's body.
Inventive Concept 144. The method according to Inventive Concept 143, wherein pulling the pulling segment of the electrosurgical slicing wire slices the resected tissue specimen within the retrieval bag into five or fewer pieces of tissue.
Inventive Concept 145. The method according to Inventive Concept 144, wherein pulling the pulling segment of the electrosurgical slicing wire slices the resected tissue specimen within the retrieval bag into three or fewer pieces of tissue.
Inventive Concept 146. The method according to Inventive Concept 145, wherein pulling the pulling segment of the electrosurgical slicing wire slices the resected tissue specimen within the retrieval bag into a single piece of tissue.
Inventive Concept 147. The method according to Inventive Concept 143, wherein pulling the pulling segment of the electrosurgical slicing wire slices the resected tissue specimen within the retrieval bag such that at least one of the pieces of tissue has a length greater than a multiple of a greatest thickness of the piece of tissue perpendicular to the length, the multiple equal to 10.
Inventive Concept 148. The method according to Inventive Concept 147, wherein the multiple is equal to 20.
Inventive Concept 149. A method for removing a resected tissue specimen from a body of a patient, the method comprising:
   inserting, into the patient's body, a retrieval bag of a tissue removal apparatus, wherein the tissue removal apparatus further includes (a) an electrosurgical slicing wire that includes a slicing segment detachably coupled to a wall of the retrieval bag, and a pulling segment free of the wall of the retrieval bag and extending out of an opening of the retrieval bag, and (b) a return electrode;
   inserting the resected tissue specimen into the retrieval bag via the opening, while the retrieval bag is in the patient's body;
   pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, thereby detaching successive longitudinal portions of the slicing segment of the electrosurgical slicing wire from the wall of the retrieval bag and slicing the resected tissue specimen within the retrieval bag into one or more pieces of tissue, at least one of which has a length greater than a multiple of a greatest thickness of the piece of tissue perpendicular to the length, the multiple equal to 10;
   while the retrieval bag is partially within the patient's body such that the opening of the retrieval bag is outside the patient's body, removing the one or more pieces of tissue from the retrieval bag and the patient's body; and
   thereafter, removing the retrieval bag from the patient's body.
Inventive Concept 150. The method according to Inventive Concept 149, wherein the multiple is equal to 20.
Inventive Concept 151. The method according to any one of Inventive Concepts 143 and 149, wherein the tissue removal apparatus includes exactly one electrosurgical slicing wire.
Inventive Concept 152. The method according to any one of Inventive Concepts 124-151,
   wherein the tissue removal apparatus further includes an electrically-insulating strip
   including a polymer film,
   wherein the strip is permanently coupled to an inner surface of the wall of the retrieval bag, so as to detachably couple the slicing segment to the wall of the retrieval bag, and
   wherein pulling the pulling segment of the electrosurgical slicing wire (a) detaches the successive longitudinal portions of the slicing segment from the wall of the retrieval bag by tearing the strip, (b) electrically exposes the successive longitudinal portions as they are detached from the wall, and (c) slices the resected tissue specimen within the retrieval bag.
Inventive Concept 153. The method according to Inventive Concept 152, wherein the electrically-insulating strip is configured to provide tear propagation along the electrically-insulating strip as the successive longitudinal portions of the slicing segment from the wall of the retrieval bag.
Inventive Concept 154. The method according to Inventive Concept 152,
   wherein the retrieval bag is flexible and is configured to assume compressed and expanded configurations, and
   wherein when the retrieval bag is in the expanded configuration:
      the retrieval bag defines a central longitudinal axis, and
      the strip and the slicing segment of the electrosurgical slicing wire wind around the central longitudinal axis at varying locations along the central longitudinal axis.
Inventive Concept 155. The method according to Inventive Concept 154,
   wherein the wall of the retrieval bag includes an inner layer and an outer layer, which define therebetween one or more inflatable chambers, and
   wherein when the retrieval bag is in the expanded configuration:
      the one or more inflatable chambers wind around the central longitudinal axis at varying locations along the central longitudinal axis, and
      at least a portion of the strip is permanently coupled to the inner surface of the wall of the retrieval bag along the one or more inflatable chambers that wind around the central longitudinal axis, so as to detachably couple the slicing segment to the wall of the retrieval bag along the one or more inflatable chambers that wind around the central longitudinal axis.
Inventive Concept 156. The method according to Inventive Concept 152, wherein the electrically-insulating strip includes an adhesive coated onto the polymer film.
Inventive Concept 157. The method according to Inventive Concept 156, wherein the strip is permanently coupled to the inner surface of the wall of the retrieval bag by the adhesive.
Inventive Concept 158. The method according to Inventive Concept 157, wherein the slicing segment is sandwiched between the strip and the inner surface of the wall of the retrieval bag.
Inventive Concept 159. The method according to Inventive Concept 156,
   wherein the adhesive is disposed on only a first surface of the polymer film, and
   wherein the polymer film is folded and wrapped around over the slicing segment such that two portions of the first surface are adhered to each other.
Inventive Concept 160. The method according to Inventive Concept 159, wherein a second surface of the polymer film, opposite the first surface, is permanently coupled to the inner surface of the wall of the retrieval bag, so as to detachably couple the slicing segment to the wall of the retrieval bag.
Inventive Concept 161. The method according to Inventive Concept 160, wherein the second surface of the polymer film is permanently coupled to the inner surface of the wall of the retrieval bag by polymer welding.
Inventive Concept 162. The method according to Inventive Concept 160, wherein the adhesive is a first adhesive, wherein the tissue removal apparatus further includes a second adhesive, and wherein the second surface of the polymer film is permanently coupled to the inner surface of the wall of the retrieval bag by the second adhesive.
Inventive Concept 163. The method according to Inventive Concept 152, wherein the polymer film is folded and wrapped around over the slicing segment such that two portions of a first surface of the polymer film are coupled to each other.
Inventive Concept 164. The method according to Inventive Concept 163, wherein a second surface of the polymer film, opposite the first surface, is permanently coupled to the inner surface of the wall of the retrieval bag, so as to detachably couple the slicing segment to the wall of the retrieval bag.
Inventive Concept 165. The method according to Inventive Concept 164, wherein the second surface of the polymer film is permanently coupled to the inner surface of the wall of the retrieval bag by polymer welding.
Inventive Concept 166. The method according to Inventive Concept 152, wherein the tissue removal apparatus further includes a buffer layer, which is coupled to the inner surface of the wall of the retrieval bag and the strip, so as to permanently couple the strip to the inner surface of the wall of the retrieval bag via the buffer layer.
Inventive Concept 167. The method according to Inventive Concept 166, wherein the buffer layer includes a thermally insulating material.
Inventive Concept 168. The method according to Inventive Concept 152, wherein a length of the strip is at least 100 cm, and wherein the slicing segment runs along the length of the strip.
Inventive Concept 169. The method according to Inventive Concept 152, wherein a length of the strip is greater than or equal to a length of the slicing segment, and wherein the slicing segment runs along the length of the strip.
Inventive Concept 170. The method according to any one of Inventive Concepts 124-151, wherein the wall of the retrieval bag includes an inner layer and an outer layer, which define therebetween one or more inflatable chambers.
Inventive Concept 171. The method according to Inventive Concept 170, wherein the inner layer and the outer layer are welded together at a plurality of welding locations so as to define the one or more inflatable chambers therebetween.
Inventive Concept 172. The method according to Inventive Concept 170,
   wherein the retrieval bag is flexible and is configured to assume compressed and expanded configurations, and
   wherein when the retrieval bag is in the expanded configuration:
      the retrieval bag defines a central longitudinal axis, and
      the one or more inflatable chambers wind around the central longitudinal axis at varying locations along the central longitudinal axis.
Inventive Concept 173. The method according to Inventive Concept 170,
   wherein the retrieval bag is shaped so as to define a base portion opposite the opening,
   wherein the wall includes (a) a first portion that defines the base portion, and (b) a second portion that is closer to the opening than the first portion is to the opening,
   wherein the first portion of the wall is defined by three or more triangles, which:
      have respective first base edges that are integral with the second portion of the wall,
      have respective second and third curved edges that meet at respective apexes, and
      include respective portions of the inner layer and the outer layer, which define therebetween respective portions of the one or more inflatable chambers, and
   wherein the second curved edges are fixed to the third curved edges of adjacent respective ones of the triangles, such that the triangles together define the base portion and the apexes together define a base of the base portion.
Inventive Concept 174. The method according to Inventive Concept 173,
   wherein the inner layer and the outer layer are welded together at a plurality of welding locations so as to define the one or more inflatable chambers therebetween, and
   wherein the second curved edges are welded to the third curved edges of the adjacent respective ones of the triangles.
Inventive Concept 175. The method according to any one of Inventive Concepts 124-151, wherein the slicing segment of the electrosurgical slicing wire is detachably embedded in the wall of the retrieval bag.
Inventive Concept 176. The method according to Inventive Concept 175,
   wherein the slicing segment of the electrosurgical slicing wire is detachably embedded in the wall of the retrieval bag, such that the wall electrically insulates the slicing segment of the electrosurgical slicing wire, and
   wherein pulling the pulling segment of the electrosurgical slicing wire comprises pulling the pulling segment of the electrosurgical slicing wire, during the application of RF electrosurgical power between the electrosurgical slicing wire and the return electrode, thereby detaching the successive longitudinal portions of the slicing segment from the wall of the retrieval bag, electrically exposing the successive longitudinal portions as they are detached from the wall, and slicing the resected tissue specimen within the retrieval bag.
Inventive Concept 177. The method according to any one of Inventive Concepts 142 and 176,
   wherein the wall of the retrieval bag includes an inner electrical insulation layer and an outer electrical insulation layer, disposed outward of the inner electrical insulation layer with respect to an interior of the retrieval bag,
   wherein the slicing segment of the electrosurgical slicing wire is detachably embedded in the wall of the retrieval bag between the inner electrical insulation layer and the outer electrical insulation layer, and
   wherein pulling the pulling segment of the electrosurgical slicing wire comprises pulling the pulling segment of the electrosurgical slicing wire, during the application of the RF electrosurgical power between the electrosurgical slicing wire and the return electrode, thereby pulling the successive longitudinal portions of the slicing segment of the electrosurgical slicing wire through the inner electrical insulation layer and detaching the successive longitudinal portions from the outer electrical insulation layer, thereby electrically exposing the successive longitudinal portions.
Inventive Concept 178. The method according to Inventive Concept 177, wherein a thickness of the outer electrical insulation layer is greater than a thickness of the inner electrical insulation layer.
Inventive Concept 179. The method according to Inventive Concept 178, wherein the thickness of the outer electrical insulation layer equals at least 110% of the thickness of the inner electrical insulation layer.
Inventive Concept 180. The method according to Inventive Concept 177, wherein the inner electrical insulation layer and the outer electrical insulation layer are adhered to each other.
Inventive Concept 181. The method according to any one of Inventive Concepts 142 and 176, wherein the wall of the retrieval bag includes:
   one or more electrical insulation layers that electrically insulate the slicing segment of the electrosurgical slicing wire, and
   an electrically-conductive safety layer, disposed outward of the one or more electrical insulation layers, and electrically coupled to an electrical lead that terminates outside the retrieval bag.
Inventive Concept 182. The method according to Inventive Concept 181, wherein the electrically-conductive safety layer includes an at least partially electrically conductive polymer.
Inventive Concept 183. The method according to Inventive Concept 181,
   wherein the tissue removal apparatus is an element of a tissue removal system that further includes a voltage monitoring circuit,
   wherein the method further comprises coupling the electrical lead to the voltage monitoring circuit, and
   wherein the tissue removal system is configured to cease the application of the RF electrosurgical power between the electrosurgical slicing wire and the return electrode upon sensing, by the voltage monitoring circuit, that a voltage in the electrically-conductive safety layer exceeds a threshold level indicative of electrical penetration through the one or more electrical insulation layers of the wall of the retrieval bag to the electrically-conductive safety layer.
Inventive Concept 184. The method according to any one of Inventive Concepts 142 and 176, wherein the slicing segment of the electrosurgical slicing wire is not surrounded by electrical insulation distinct from the wall of the retrieval bag.
Inventive Concept 185. The method according to any one of Inventive Concepts 142 and 176, wherein the slicing segment of the electrosurgical slicing wire is detachably embedded in a single electrical insulation layer of the wall of the retrieval bag.
Inventive Concept 186. The method according to any one of Inventive Concepts 124-185,
   wherein the pulling segment is proximal to the slicing segment of the electrosurgical slicing wire, and
   wherein pulling the pulling segment of the electrosurgical slicing wire comprises pulling the pulling segment of the electrosurgical slicing wire, thereby detaching, from the wall of the retrieval bag, the successive longitudinal portions in a proximal-to-distal direction along the slicing segment of the electrosurgical slicing wire.
Inventive Concept 187. The method according to any one of Inventive Concepts 124-186, wherein a length of the slicing segment is at least 100 cm.
Inventive Concept 188. The method according to any one of Inventive Concepts 124-187, wherein a length of the slicing segment of the electrosurgical slicing wire equals at least 4 times a perimeter of the opening of the retrieval bag.
Inventive Concept 189. The method according to any one of Inventive Concepts 130-151 and 177-188,
   wherein the retrieval bag is flexible,
   wherein inserting comprises inserting, into the patient's body, the flexible retrieval bag while the retrieval bag is in a compressed configuration, and
   wherein the retrieval bag is configured to assume an expanded configuration in which the retrieval bag defines a central longitudinal axis, and the slicing segment of the electrosurgical slicing wire winds around the central longitudinal axis at varying locations along the central longitudinal axis.
Inventive Concept 190. The method according to any one of Inventive Concepts 124 and 189, wherein the slicing segment of the electrosurgical slicing wire winds around the central longitudinal axis in at least 4 turns.
Inventive Concept 191. The method according to any one of Inventive Concepts 124 and 189, wherein when the retrieval bag is in the expanded configuration:
   the opening is at a proximal end of the retrieval bag opposite a distal end of the retrieval bag at a base of the retrieval bag, and
   (a) a first location on the central longitudinal axis corresponding to the opening of the retrieval bag is at least 5 cm from (b) a second location on the central longitudinal axis corresponding to a proximal-most location on the wall of the retrieval bag at which the slicing segment is detachably coupled to the wall.
Inventive Concept 192. The method according to any one of Inventive Concepts 124 and 189, wherein the retrieval bag is shaped so as to define a base opposite the opening, and wherein the slicing segment of the electrosurgical slicing wire winds around the central longitudinal axis in a base-to-opening direction at a nondecreasing distance from the central longitudinal axis when the retrieval bag is in the expanded configuration.
Inventive Concept 193. The method according to Inventive Concept 192, wherein the slicing segment of the electrosurgical slicing wire is shaped as a generally three-dimensional spiral when the retrieval bag is in the expanded configuration.
Inventive Concept 194. The method according to any one of Inventive Concepts 130-151 and 177-188,
   wherein the retrieval bag is flexible,
   wherein inserting comprises inserting, into the patient's body, the flexible retrieval bag while the retrieval bag is in a compressed configuration, and
   wherein the retrieval bag is configured to assume an expanded configuration in which the retrieval bag defines a central longitudinal axis, and
   wherein the slicing segment of the electrosurgical slicing wire is detachably coupled to the wall of the retrieval bag such that the slicing segment travels around the central longitudinal axis in at least four turns in alternating clockwise and counterclockwise directions in axially sequential turns when the retrieval bag is in the expanded configuration.
Inventive Concept 195. The method according to any one of Inventive Concepts 124-194,
   wherein the tissue removal apparatus further includes a containment bag within which the retrieval bag is disposed, so as to define one or more inflatable chambers between an inner surface of the containment bag and an outer surface of the retrieval bag,
   wherein inserting the retrieval bag into the patient's body comprises inserting the retrieval bag and the containment bag into the patient's body, and
   wherein the method further comprises, after inserting the retrieval bag and the containment bag into the patient's body, inflating the one or more inflatable chambers with a fluid.
Inventive Concept 196. The method according to Inventive Concept 195,
   wherein a wall of the containment bag is shaped so as to define a plurality of inflatable compartments arranged entirely around the containment bag, and
   wherein the method further comprises, after inserting the retrieval bag into the patient's body, inflating the plurality of inflatable compartments with a fluid.
Inventive Concept 197. The method according to Inventive Concept 196,
   wherein the retrieval bag is flexible and is configured to assume compressed and expanded configurations, and to define a central longitudinal axis when the retrieval bag is in the expanded configuration, and
   wherein (a) a location along the central longitudinal axis corresponding to a proximal edge of the plurality of inflatable compartments is at or proximal to (b) a location along the central longitudinal axis corresponding to a proximal-most point at which the slicing segment of the electrosurgical slicing wire is detachably coupled to the wall of the retrieval bag, when the retrieval bag is in the expanded configuration.
Inventive Concept 198. The method according to Inventive Concept 195, wherein the containment bag includes a camera port open to one of the one or more inflatable chambers, and wherein the method further comprises inserting a camera through the camera port.
Inventive Concept 199. The method according to any one of Inventive Concepts 124-198, wherein the return electrode is coupled to the retrieval bag.
Inventive Concept 200. The method according to any one of Inventive Concepts 124-199,
   wherein the tissue removal apparatus is an element of a tissue removal system that further includes a control handle, which is shaped so as to define an elongate insertion tip, and
   wherein the method further comprises, before pulling the pulling segment of the electrosurgical slicing wire, inserting the elongate insertion tip into the patient's body and into the retrieval bag via the opening, while the pulling segment of the electrosurgical slicing wire passes through a channel within the elongate insertion tip.
Inventive Concept 201. The method according to Inventive Concept 200, inserting the elongate insertion tip comprises inserting the elongate insertion tip into the patient's body and into the retrieval bag until the elongate insertion tip comes in physical contact with the resected tissue specimen.
Inventive Concept 202. The method according to any one of Inventive Concepts 124-201, wherein the tissue removal apparatus is an element of a tissue removal system that further includes a control handle, which includes the return electrode.
Inventive Concept 203. The method according to any one of Inventive Concepts 124-202,
   wherein the tissue removal apparatus is an element of a tissue removal system that further includes a control handle,
   wherein the pulling segment of the electrosurgical slicing wire is electrically and mechanically couplable to the control handle, and
   wherein pulling the pulling segment of the electrosurgical slicing wire comprises activating a motor of the control handle to pull the pulling segment of the electrosurgical slicing wire during the application of RF electrosurgical power between the electrosurgical slicing wire and the return electrode, when the pulling segment of the electrosurgical slicing wire is electrically and mechanically coupled to the control handle.
Inventive Concept 204. The method according to Inventive Concept 203, wherein the control handle includes a user control, and wherein pulling the pulling segment of the electrosurgical slicing wire comprises actuating the user control to activate the control handle in:
   a first step, in which the control handle activates the motor to pull the pulling segment of the electrosurgical slicing wire without applying the RF electrosurgical power to the electrosurgical slicing wire, and
   a second step, in which the control handle, in addition to activating the motor to pull the pulling segment of the electrosurgical slicing wire, applies the RF electrosurgical power to the pulling segment.
Inventive Concept 205. The method according to Inventive Concept 204, wherein actuating the user control to activate the control handle in the first step activates the motor to pull the pulling segment of the electrosurgical slicing wire without applying the RF electrosurgical power to the electrosurgical slicing wire, until a longitudinal portion of the slicing segment of the electrosurgical slicing wire comes detached from the wall of the retrieval bag.
Inventive Concept 206. The method according to any one of Inventive Concepts 124-205, further comprising filling at least a portion of empty spaces between the resected tissue specimen and an inner surface of the retrieval bag with a conductive gel, when the resected tissue specimen is within the retrieval bag.
Inventive Concept 207. The method according to any one of Inventive Concepts 124-206, wherein the resected tissue specimen is a resected uterus, and wherein inserting the resected tissue specimen into the retrieval bag comprises inserting the resected uterus into the retrieval bag.
Inventive Concept 208. The method according to any one of Inventive Concepts 124-207, wherein the resected tissue specimen is a resected fibroid, and wherein inserting the resected tissue specimen into the retrieval bag comprises inserting the resected fibroid into the retrieval bag.
Inventive Concept 209. The method according to any one of Inventive Concepts 124-208, wherein the resected tissue specimen is all or a portion of an organ, and wherein inserting the resected tissue specimen into the retrieval bag comprises inserting all or a portion of an organ into the retrieval bag.
Inventive Concept 210. The method according to Inventive Concept 209, wherein the organ is selected from the group of organs consisting of: a resected spleen, a resected kidney, and a resected lung.
Inventive Concept 211. A method for removing a resected tissue specimen from a body of a patient, the method comprising:
   inserting, into the patient's body, a retrieval bag of a tissue removal apparatus, wherein the tissue removal apparatus further includes (a) an electrosurgical electrode and (b) a return electrode, wherein the retrieval bag includes a wall including: (i) one or more electrical insulation layers, which electrically isolate at least a portion of the electrosurgical electrode from portions of the patient's body outside the retrieval bag, when the retrieval bag is disposed partially within the patient's body; and (ii) an electrically-conductive safety layer, disposed outward of the one or more electrical insulation layers, and electrically coupled to an electrical lead that terminates outside the retrieval bag;
   inserting the resected tissue specimen into the retrieval bag via an opening of the retrieval bag, while the retrieval bag is in the patient's body;
   during application of RF electrosurgical power between the electrosurgical electrode and the return electrode when the resected tissue specimen is within the retrieval bag and the return electrode is in electrical contact with the resected tissue specimen, using the electrosurgical electrode to cut the resected tissue specimen within the retrieval bag;
   while the retrieval bag is partially within the patient's body such that the opening of the retrieval bag is outside the patient's body, removing the cut resected tissue specimen from the retrieval bag and the patient's body; and
   thereafter, removing the retrieval bag from the patient's body.
Inventive Concept 212. The method according to Inventive Concept 211, wherein the electrically-conductive safety layer includes an at least partially electrically conductive polymer.
Inventive Concept 213. The method according to Inventive Concept 211,
   wherein the tissue removal apparatus is an element of a tissue removal system that further includes a voltage monitoring circuit,
   wherein the method further comprises coupling the electrical lead to the voltage monitoring circuit, and
   wherein the tissue removal system is configured to cease the application of the RF electrosurgical power between the electrosurgical slicing wire and the return electrode upon sensing, by the voltage monitoring circuit, that a voltage in the electrically-conductive safety layer exceeds a threshold level indicative of electrical penetration through the one or more electrical insulation layers of the wall of the retrieval bag to the electrically-conductive safety layer.
Inventive Concept 214. The method according to Inventive Concept 211, wherein the electrosurgical electrode includes an electrosurgical slicing wire.
Inventive Concept 215. A method for removing a resected tissue specimen from a body of a patient, the method comprising:
   inserting, into the patient's body, a retrieval bag and a containment bag of a tissue removal apparatus, wherein the tissue removal apparatus further includes one or more tissue cutters detachably coupled to a wall of the retrieval bag, and wherein the retrieval bag is disposed within the containment bag, so as to define one or more inflatable chambers between an inner surface of the containment bag and an outer surface of the retrieval bag;
   inserting the resected tissue specimen into the retrieval bag via an opening of the retrieval bag, while the retrieval bag and the containment bag are in the patient's body;
   inflating the one or more inflatable chambers with a fluid;
   thereafter, using the one or more tissue cutters to cut the resected tissue specimen when the resected tissue specimen is within the retrieval bag;
   while the retrieval bag is partially within the patient's body such that the opening of the retrieval bag is outside the patient's body, removing the cut resected tissue specimen from the retrieval bag and the patient's body; and
   thereafter, removing the retrieval bag and the containment bag from the patient's body.
Inventive Concept 216. The method according to Inventive Concept 215, wherein the one or more tissue cutters include one or more cutting wires.
Inventive Concept 217. The method according to Inventive Concept 215,
   wherein a wall of the containment bag is shaped so as to define a plurality of inflatable compartments arranged entirely around the containment bag, and
   wherein the method further comprises inflating the plurality of inflatable compartments with a fluid.
Inventive Concept 218. The method according to Inventive Concept 217,
   wherein the retrieval bag is flexible and is configured to assume compressed and expanded configurations, and to define a central longitudinal axis when the retrieval bag is in the expanded configuration, and
   wherein (a) a location along the central longitudinal axis corresponding to a proximal edge of the plurality of inflatable compartments is at or proximal to (b) a location along the central longitudinal axis corresponding to a proximal-most point at which the one or more tissue cutters are detachably coupled to the wall of the retrieval bag, when the retrieval bag is in the expanded configuration.
Inventive Concept 219. The method according to Inventive Concept 215, wherein the containment bag includes a camera port open to one of the one or more inflatable chambers, and wherein the method further comprises inserting a camera through the camera port.
Inventive Concept 220. The method according to any one of Inventive Concepts 215-219, further comprising coupling the one or more tissue cutters to a generator via one or more electrical leads.
Inventive Concept 221. A method for removing a resected tissue specimen from a body of a patient, the method comprising:
   inserting, into the patient's body, a retrieval bag of a tissue removal apparatus, wherein the tissue removal apparatus further includes (a) one or more tissue cutters detachably coupled to a wall of the retrieval bag, and (b) a return electrode;
   inserting the resected tissue specimen into the retrieval bag via an opening of the retrieval bag, while the retrieval bag is in the patient's body;
   filling at least a portion of empty spaces between the resected tissue specimen and an inner surface of the retrieval bag with a conductive gel, when the resected tissue specimen is within the retrieval bag;
   thereafter, using the one or more tissue cutters to cut the resected tissue specimen when the resected tissue specimen is within the retrieval bag;
   while the retrieval bag is partially within the patient's body such that the opening of the retrieval bag is outside the patient's body, removing the cut resected tissue specimen from the retrieval bag and the patient's body; and
   thereafter, removing the retrieval bag from the patient's body.
Inventive Concept 222. The method according to Inventive Concept 221, wherein the one or more tissue cutters include one or more cutting wires.
Inventive Concept 223. The method according to Inventive Concept 221, further comprising coupling the one or more tissue cutters to a generator via one or more electrical leads.
Inventive Concept 224. A method for removing a resected tissue specimen from a body of a patient, the method comprising:
   inserting, into the patient's body, a retrieval bag of a tissue removal apparatus, wherein (a) the retrieval bag is shaped so as to define an opening for receiving the resected tissue specimen within the patient's body, and a base opposite the opening, (b) the retrieval bag is flexible and is configured to assume compressed and expanded configurations, and to define a central longitudinal axis when the retrieval bag is in the expanded configuration, and (c) an annular inflatable positioning chamber is fixed to and encircles an axial portion of an external surface of the retrieval bag, the axial portion axially between the opening and the base, spaced away from the opening and from the base;
   inserting the resected tissue specimen into the retrieval bag via the opening, while the retrieval bag is in the patient's body, and
   thereafter, pulling out of the patient's body via a laparoscopic port, a proximal portion of the retrieval bag that defines the opening and is proximal to the annular inflatable positioning chamber, such that the annular inflatable positioning chamber remains within the patient's body.
Inventive Concept 225. The method according to Inventive Concept 224,
   wherein the tissue removal apparatus further includes (a) an electrosurgical slicing wire that includes a slicing segment and (b) a return electrode,
   wherein the slicing segment of the electrosurgical slicing wire is detachably coupled to a wall of the retrieval bag,
   wherein the electrosurgical slicing wire further includes a pulling segment free of the wall of the retrieval bag and extending out of the opening of the retrieval bag, and
   wherein the method further comprises, after pulling the proximal portion of the retrieval bag out of the patient's body via the laparoscopic port, pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, thereby detaching successive longitudinal portions of the slicing segment of the electrosurgical slicing wire from the wall of the retrieval bag and slicing the resected tissue specimen within the retrieval bag.
Inventive Concept 226. A method for removing a resected tissue specimen from a body of a patient, the method comprising:
   inserting, into the patient's body, a retrieval bag of a tissue removal apparatus, wherein (a) the retrieval bag is shaped so as to define a proximal opening for receiving the resected tissue specimen within the patient's body, and a distal base portion opposite the opening, (b) a drainage chamber is disposed distal to the distal base portion, and (c) a wall of the distal base portion of the retrieval bag is shaped so as to define one or more drainage openings between an interior of the retrieval bag and the drainage chamber; and
   inserting the resected tissue specimen into the retrieval bag via the opening, while the retrieval bag is in the patient's body.
Inventive Concept 227. The method according to Inventive Concept 226, further comprising suctioning, via a tube coupled in fluid communication with the drainage chamber, liquid that drained from the interior of the retrieval bag to the drainage chamber.
Inventive Concept 228. The method according to Inventive Concept 226,
   wherein the tissue removal apparatus further includes (a) an electrosurgical slicing wire that includes a slicing segment and (b) a return electrode,
   wherein the slicing segment of the electrosurgical slicing wire is detachably coupled to a wall of the retrieval bag,
   wherein the electrosurgical slicing wire further includes a pulling segment free of the wall of the retrieval bag and extending out of the opening of the retrieval bag, and
   wherein the method further comprises, after inserting the resected tissue specimen into the retrieval bag, pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, thereby detaching successive longitudinal portions of the slicing segment of the electrosurgical slicing wire from the wall of the retrieval bag and slicing the resected tissue specimen within the retrieval bag.
Inventive Concept 229. A tissue removal apparatus for removing a resected tissue specimen from a body of a patient, the tissue removal apparatus comprising:
   a retrieval bag having an opening for receiving the resected tissue specimen within the patient's body, wherein the retrieval bag is flexible and is configured to assume compressed and expanded configurations; and
   a slicing wire that includes a slicing segment detachably coupled to a wall of the retrieval bag, and a pulling segment free of the wall of the retrieval bag and extending out of the opening of the retrieval bag,
   wherein when the retrieval bag is in the expanded configuration:
      the retrieval bag defines a central longitudinal axis, and
      the slicing segment of the slicing wire winds around the central longitudinal axis at varying locations along the central longitudinal axis, and
   wherein the tissue removal apparatus is configured such that pulling the pulling segment of the slicing wire, when the resected tissue specimen is within the retrieval bag, detaches successive longitudinal portions of the slicing segment of the slicing wire from the wall of the retrieval bag and slices the resected tissue specimen within the retrieval bag.
Inventive Concept 230. The apparatus according to Inventive Concept 229, wherein the tissue removal apparatus comprises exactly one slicing wire, which is single-stranded or multi-stranded.
Inventive Concept 231. The apparatus according to Inventive Concept 229, wherein the tissue removal apparatus is configured such that the pulling of the pulling segment of the slicing wire detaches the successive longitudinal portions of the slicing segment of the slicing wire from the wall of the retrieval bag and slices the resected tissue specimen within the retrieval bag into ten or fewer pieces of tissue.
Inventive Concept 232. A tissue removal apparatus for removing a resected tissue specimen from a body of a patient, the tissue removal apparatus comprising:
   a retrieval bag having an opening for receiving the resected tissue specimen within the patient's body, and
   exactly one slicing wire that includes a slicing segment detachably coupled to a wall of the retrieval bag, and a pulling segment free of the wall of the retrieval bag and extending out of the opening of the retrieval bag, wherein the wire is single-stranded or multi-stranded,
   wherein the tissue removal apparatus is configured such that pulling the pulling segment of the slicing wire, when the resected tissue specimen is within the retrieval bag, detaches successive longitudinal portions of the slicing segment of the slicing wire from the wall of the retrieval bag and slices the resected tissue specimen within the retrieval bag.
Inventive Concept 233. The apparatus according to Inventive Concept 232, wherein the wire is multi-stranded and braided.

The present invention will be more fully understood from the following detailed description of embodiments thereof, taken together with the drawings, in which:

### BRIEF DESCRIPTION OF THE DRAWINGS

FIG. 1 is a schematic illustration of a tissue removal system being used for removing a resected tissue specimen from a body of a patient, in accordance with an application of the present invention;

FIG. 2A is a schematic illustration of certain elements of the tissue removal system of FIG. 1 assembled and in an exploded view, in accordance with an application of the present invention;

FIGS. 2B and 2C are schematic illustrations of respective configurations of certain elements of the tissue removal system of FIG. 1, in accordance with respective applications of the present invention;

FIG. 2D is a schematic illustration of a portion of pulling of a pulling segment of an electrosurgical slicing wire of the tissue removal system of FIG. 1, in accordance with an application of the present invention;

FIGS. 2E-G are schematic illustrations of respective configurations of certain elements of a tissue removal apparatus, in accordance with respective applications of the present invention;

FIGS. 3A-D are schematic illustrations of an electrosurgical slicing wire of the tissue removal system of FIG. 1, in accordance with an application of the present invention;

FIGS. 4A-B are schematic illustrations of an alterative configuration of the tissue removal system of FIG. 1 in which the system further comprises a containment bag, in accordance with respective applications of the present invention;

FIG. 5 is a schematic illustration of another alterative configuration of the tissue removal system of FIG. 1 in which the system further comprises a containment bag, in accordance with respective applications of the present invention;

FIGS. 6A-B are schematic isometric and cross-sectional views of another configuration of a retrieval bag, in accordance with an application of the present invention;

FIGS. 7A-B are schematic illustrations of respective configurations of a wall of the retrieval bag of FIGS. 6A-B and certain other elements of a tissue removal apparatus, in accordance with respective applications of the present invention;

FIG. 8 is a schematic illustration of a welding pattern applied to a retrieval bag, in accordance with an application of the present invention;

FIG. 9 is a schematic illustration of an alternative configuration of a tissue removal apparatus, in accordance with an application of the present invention;

FIGS. 10A-B are schematic illustrations of alternative configurations of a tissue removal apparatus, in accordance with respective applications of the present invention;

FIG. 11 is a schematic illustration of an alternative configuration of a tissue removal apparatus, in accordance with an application of the present invention;

FIGS. 12A-B are schematic illustrations of an alternative arrangement of a slicing segment of an electrosurgical slicing wire of the tissue removal system of FIG. 1 detachably coupled to a wall of a retrieval bag of the system, in accordance with an application of the present invention;

FIGS. 13A-B are schematic illustrations of another alternative arrangement of a slicing segment of an electrosurgical slicing wire of the tissue removal system of FIG. 1 detachably coupled to a wall of a retrieval bag of the system, in accordance with an application of the present invention;

FIGS. 14A-B are schematic illustrations of yet another alternative arrangement of a slicing segment of an electrosurgical slicing wire of the tissue removal system of FIG. 1 detachably coupled to a wall of a retrieval bag of the system, in accordance with an application of the present invention;

FIGS. 15A and 15B-C are schematic illustrations of a control handle of the tissue removal system of FIG. 1, in accordance with respective applications of the present invention;

FIGS. 16A-J are schematic illustrations of a method for removing a resected tissue specimen from a body of a patient, in accordance with an application of the present invention;

FIGS. 17A-B are schematic illustrations of a portion of a method for removing a resected tissue specimen from a body of a patient, in accordance with an application of the present invention; and

FIGS. 18A-C are photographs of an experiment conducted by some of the inventors to test certain elements of the apparatus and methods described herein.

### DETAILED DESCRIPTION OF APPLICATIONS

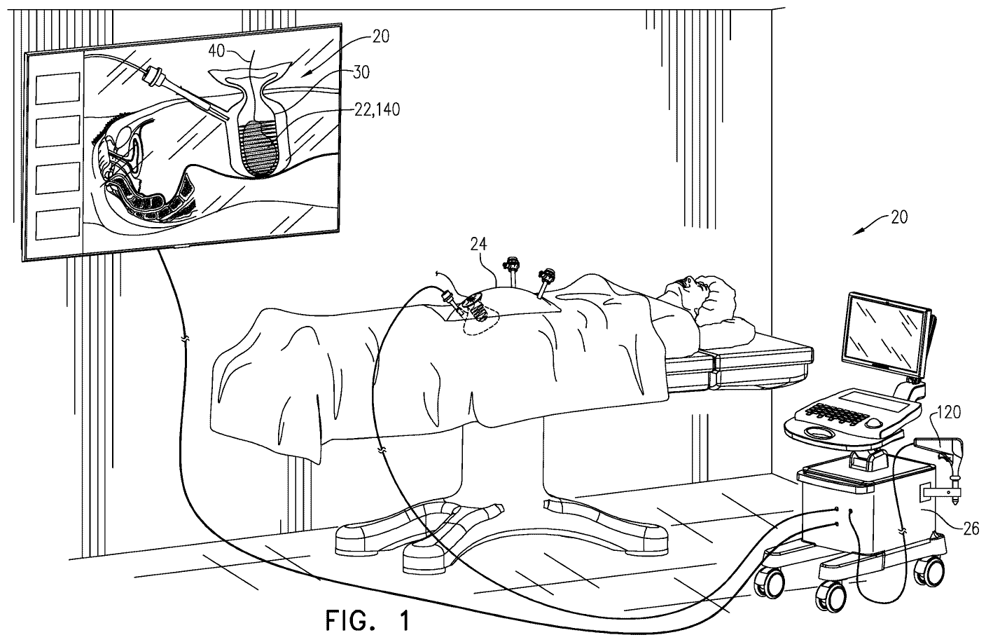

FIG. 1 is a schematic illustration of a tissue removal system 10 being used for removing a resected tissue specimen 22 from a body 24 of a patient, in accordance with an application of the present invention. Tissue removal system 10 is typically for use with an RF electrosurgical generator (ESG) 26, as known in the art and widely available. For example, resected tissue specimen 22 may be all or a portion of an organ, such as a uterus (as shown in some of the figures), a spleen, a kidney, or a lung, or another tissue, such as a fibroid. Tissue removal system 10 may be used to treat a human or a non-human (animal) patient.

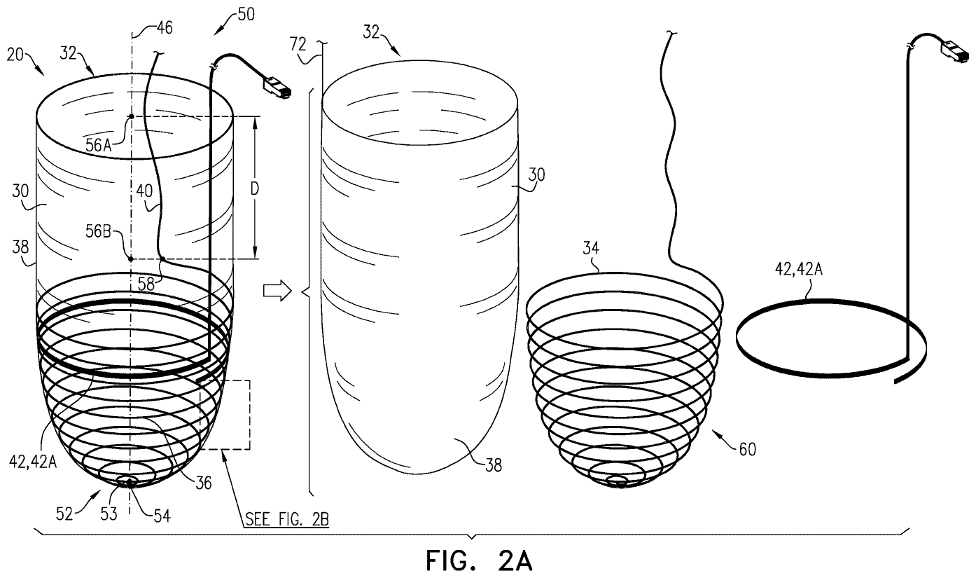

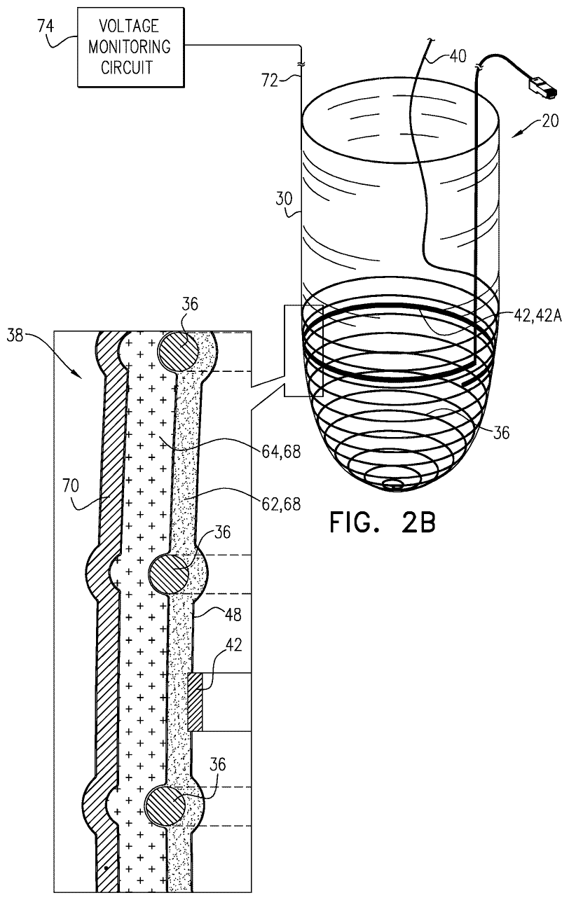

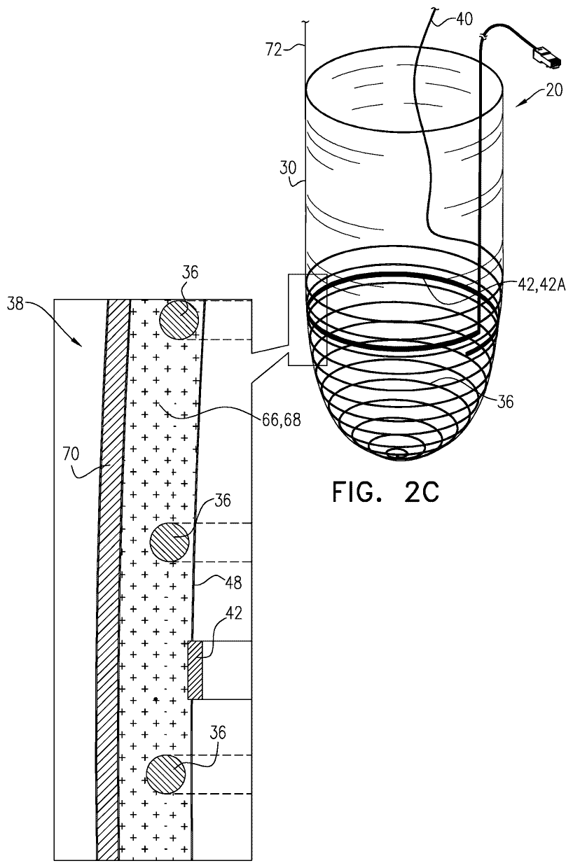

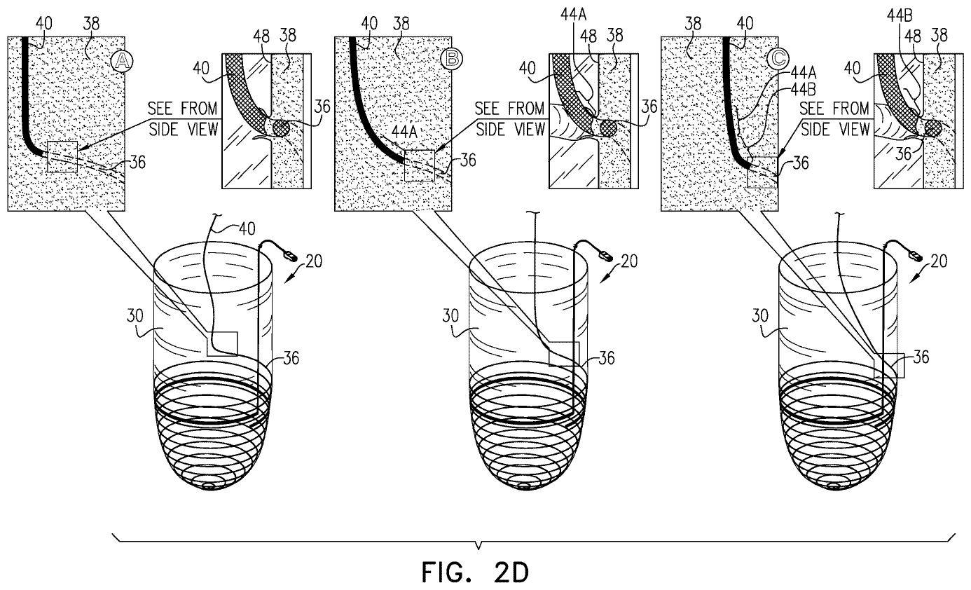

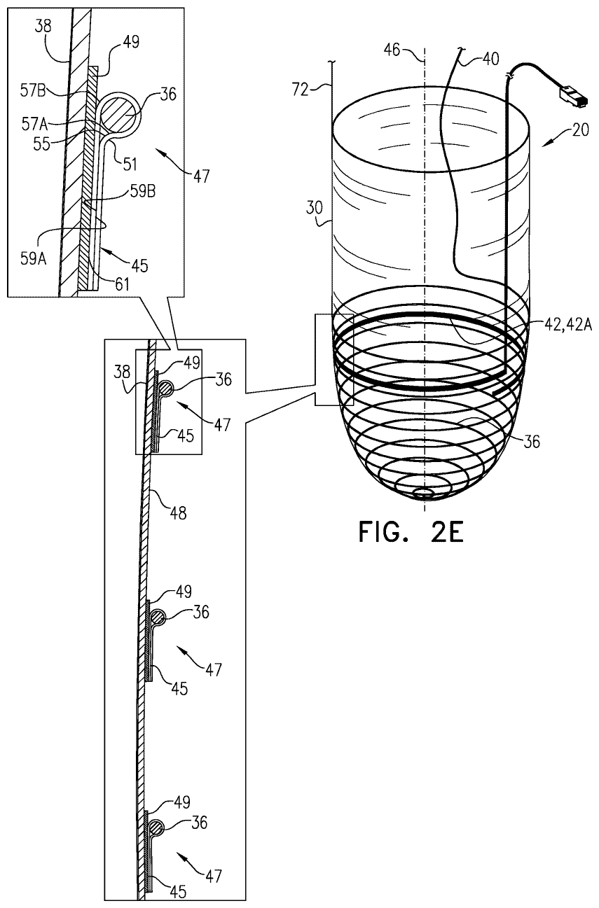

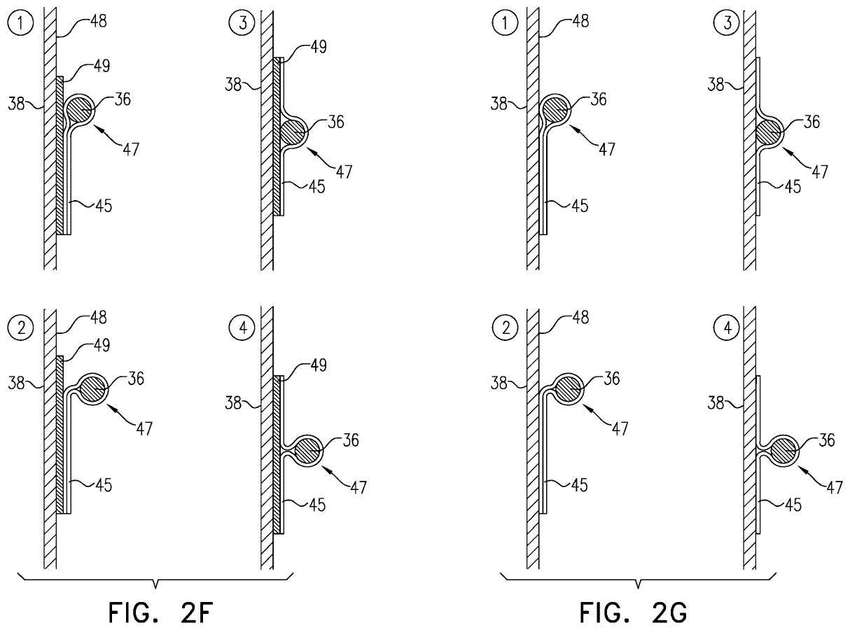

Reference is also made to FIG. 2A, which is a schematic illustration of a tissue removal apparatus 20 of tissue removal system 10 assembled and in an exploded view, in accordance with an application of the present invention.

Reference is further made to FIGS. 2B and 2C, which are schematic illustrations of respective configurations of certain elements of tissue removal apparatus 20, in accordance with respective applications of the present invention.

For some applications, tissue removal apparatus 20 comprises:
a retrieval bag 30 having an opening 32 for receiving resected tissue specimen 22 within the patient's body 24;
an electrosurgical slicing wire 34 that includes a slicing segment 36 detachably coupled to a wall 38 of retrieval bag 30, and a pulling segment 40 free of wall 38 of retrieval bag 30 and extending out of opening 32 of retrieval bag 30; and
a return electrode 42, which is configured to be brought into contact with resected tissue specimen 22, typically direct physical contact with resected tissue specimen 22, when resected tissue specimen 22 is within retrieval bag 30.

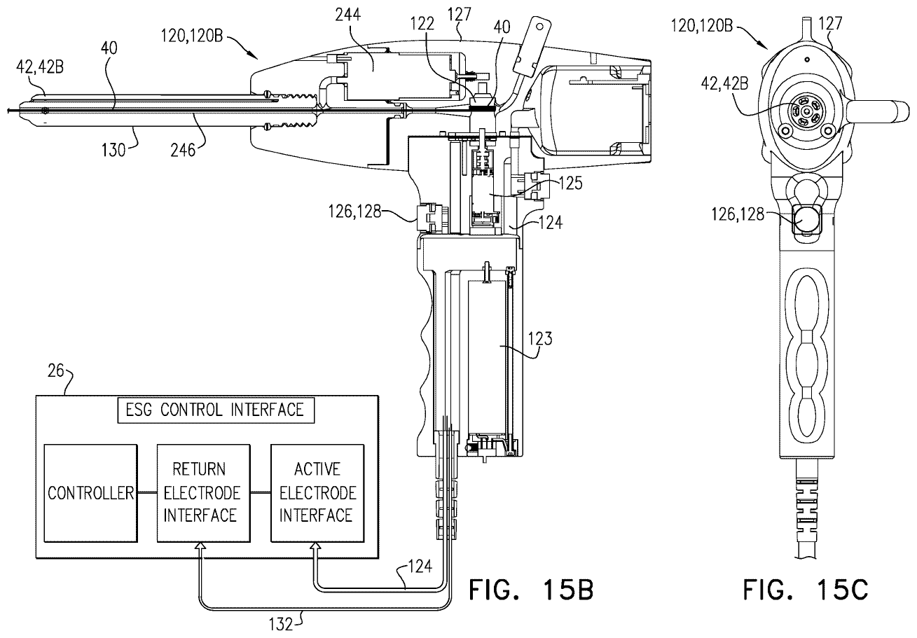

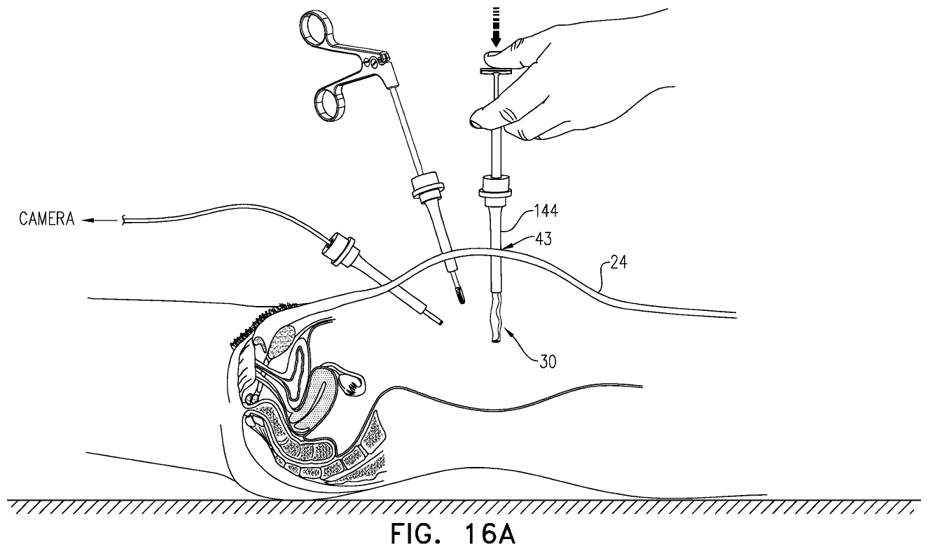

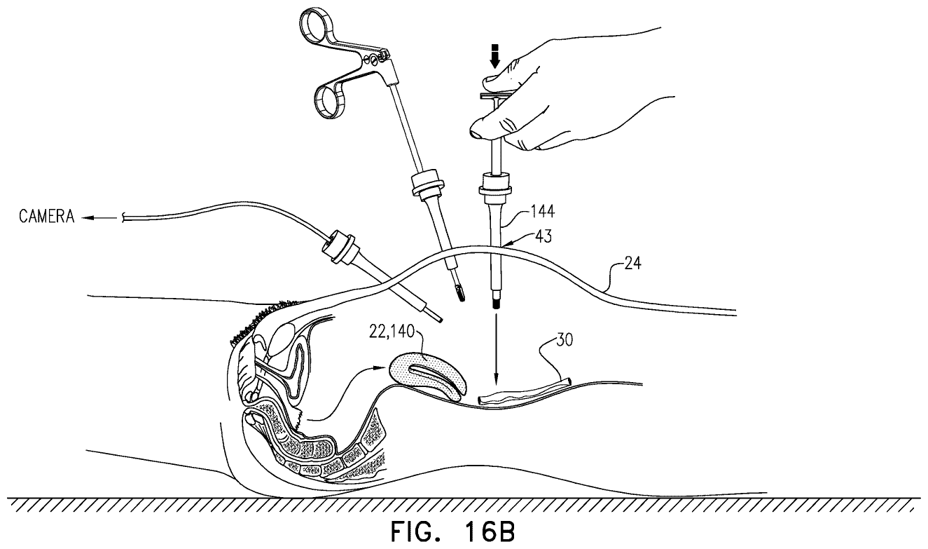

(Although pulling segment 40 is free of wall 38 of retrieval bag 30, pulling segment 40 may be otherwise constrained, such as by a control handle 120, e.g., elongate insertion tip 130 thereof, described hereinbelow with reference to FIGS. 15A-C. For example, a portion of pulling segment 40 that extends out of opening 32 of retrieval bag 30 may be positioned within elongate insertion tip 130.)

Electrosurgical slicing wire 34 (typically a proximal end of pulling segment 40 thereof) is typically electrically couplable to an active electrode receptacle of RF ESG 26, typically a monopolar active receptacle, optionally via control handle 120, as described hereinbelow with reference to FIGS. 15A-C. Optionally, both the proximal end of pulling segment 40 and a distal end of slicing segment 36 of electrosurgical slicing wire 34 are electrically couplable to the active electrode receptacle of RF ESG 26, typically a monopolar active receptacle.

Return electrode 42 (also known in the art as a passive electrode) is typically electrically couplable to a return electrode receptacle of RF ESG 26, optionally via control handle 120, as described hereinbelow with reference to FIGS. 15A-C. Optionally, return electrode 42 may implement the features of return electrode 242, described hereinbelow with reference to FIG. 11.

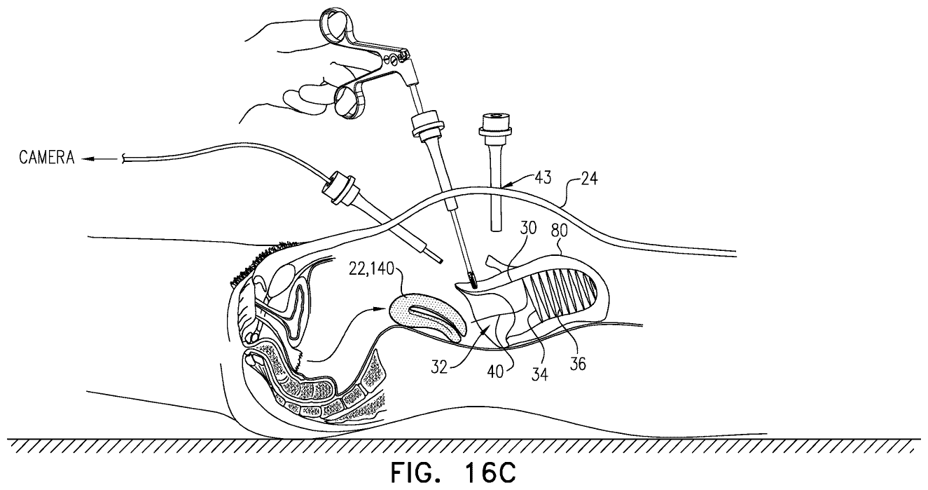

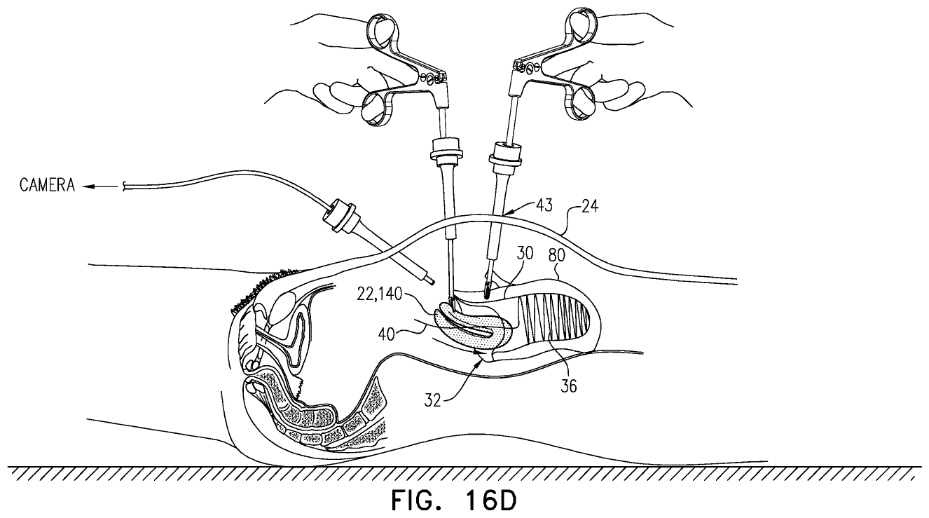

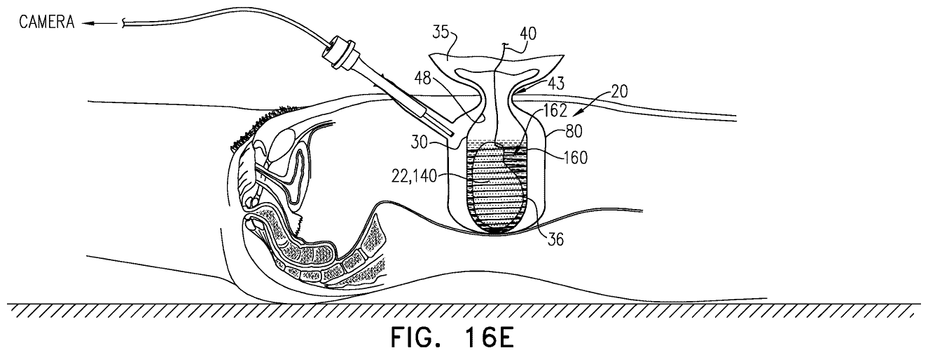

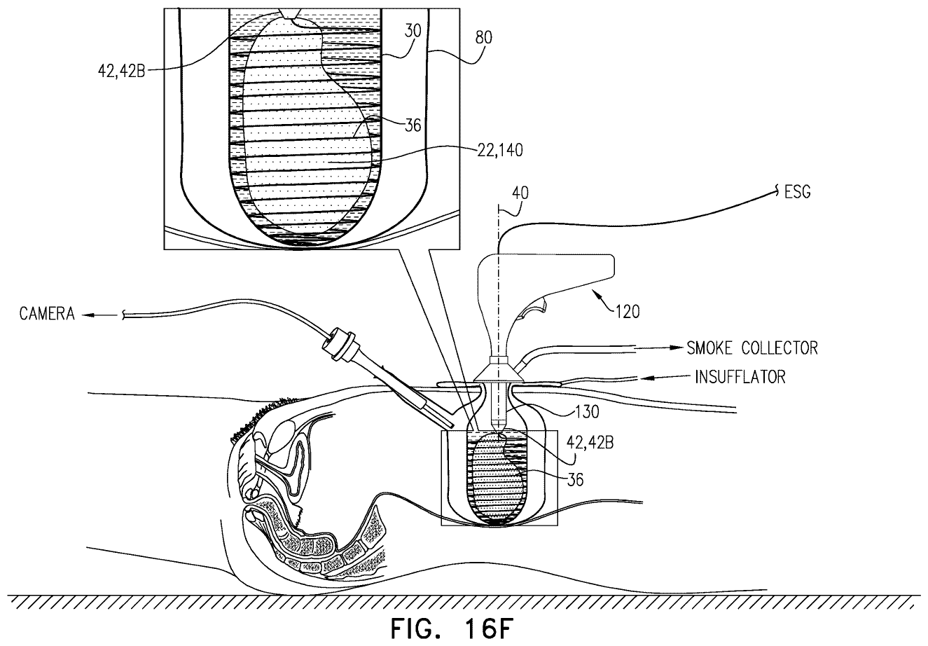

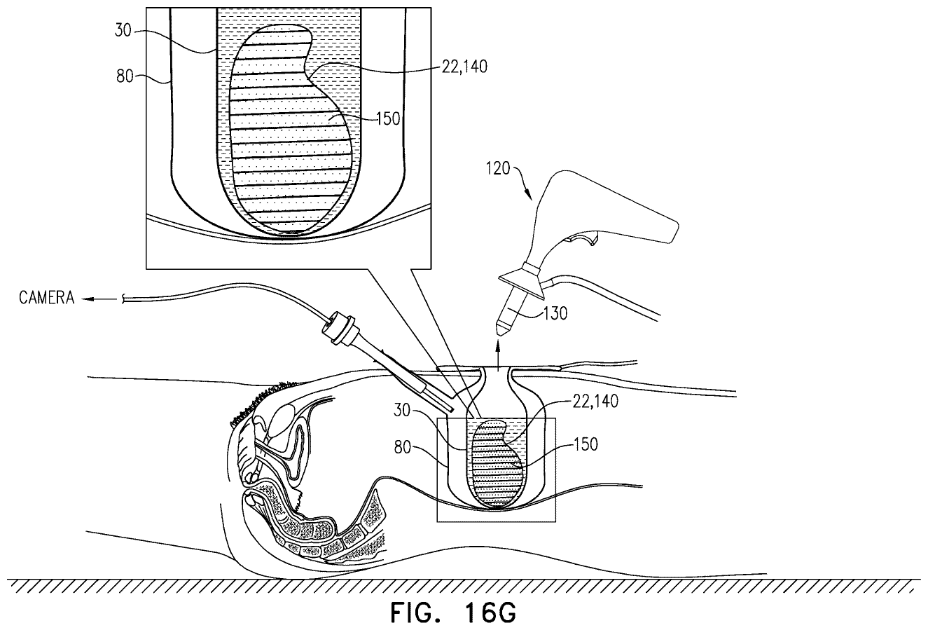

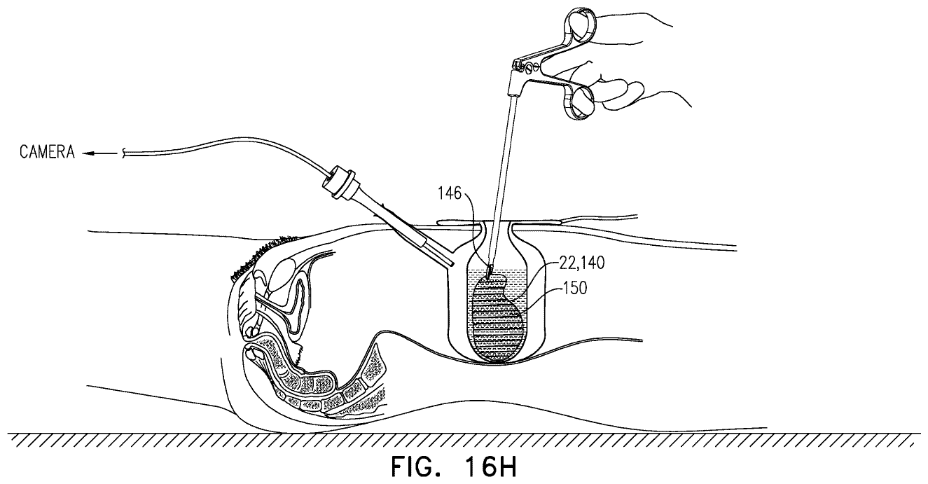

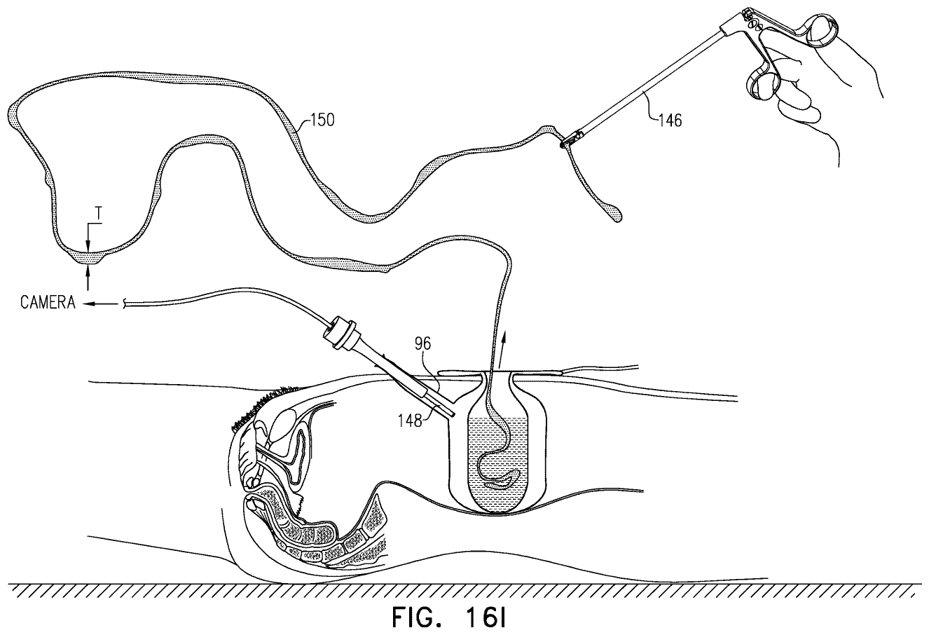

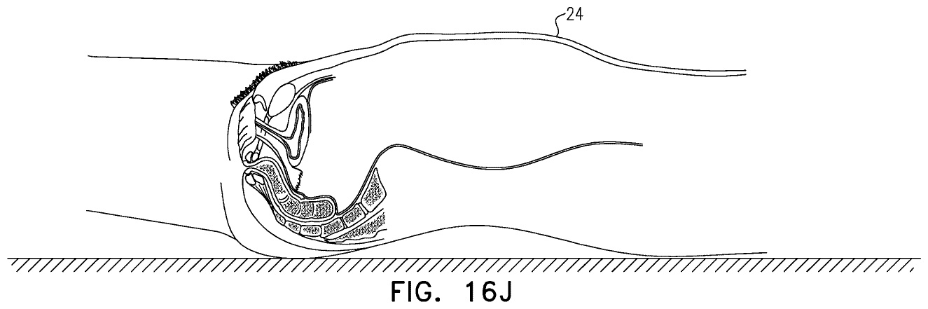

Tissue removal apparatus 20 is configured such that pulling pulling segment 40 of electrosurgical slicing wire 34, during application of RF electrosurgical power between electrosurgical slicing wire 34 and return electrode 42 when resected tissue specimen 22 is within retrieval bag 30 and return electrode 42 is in electrical contact with resected tissue specimen 22, detaches successive longitudinal portions of slicing segment 36 of electrosurgical slicing wire 34 from wall 38 of retrieval bag 30 and slices resected tissue specimen 22 within retrieval bag 30. This slicing is described in detail hereinbelow with reference to the transition between FIG. 16F and FIG. 16G.

(Pulling segment 40 is proximal to slicing segment 36 along electrosurgical slicing wire 34.)

Typically, retrieval bag 30 is flexible and is configured to assume compressed and expanded configurations.

Optionally, retrieval bag 30 is provided with a plurality of sizes and/or shapes in order to accommodate different sizes and shapes of various organs.

For some applications, a volume of retrieval bag 30 when fully expanded is 500-2,000 ml, such as 1,000-1,500 ml.

For some applications, tissue removal apparatus 20 comprises exactly one electrosurgical slicing wire 34 that includes slicing segment 36 detachably coupled to wall 38 of retrieval bag 30, and pulling segment 40 free of wall 38 of retrieval bag 30 and extending out of opening 32 of retrieval bag 30. The exactly one electrosurgical slicing wire 34 is single-stranded or multi-stranded, as is known in the general wire art. Optionally, each strand comprises filaments, as is known in the general wire art. In configurations in which the exactly one electrosurgical slicing wire 34 is multi-stranded, the strands may be braided or non-braided. Alternatively, tissue removal apparatus 20 comprises two or more electrosurgical slicing wires 34 that include respective slicing segments 36 detachably coupled to wall 38 of retrieval bag 30, and respective pulling segments 40 free of wall 38 of retrieval bag 30 and extending out of opening 32 of retrieval bag 30.

Typically, electrosurgical slicing wire 34 comprises metal, such as tungsten, stainless steel, platinum, titanium, or related alloys. For example, the wire may have a diameter of 0.02-0.6 mm, such as 0.05-0.15 mm (e.g., 0.09 mm).

Typically, return electrode 42 comprises metal, such as copper, silver, tungsten, stainless steel, platinum, titanium, or related alloys.

For some applications, return electrode 42 is a return electrode 42A coupled to retrieval bag 30, such as an inner surface 48 of retrieval bag 30, such as shown in FIGS. 1-2C and labeled in FIGS. 2B and 2C. This location brings return electrode 42, 42A into electrical contact with resected tissue specimen 22, typically direct physical contact with resected tissue specimen 22, when resected tissue specimen 22 is within retrieval bag 30.

Reference is still made to FIGS. 1-2C, and is additionally made to FIG. 2D, which is a schematic illustration of a portion of pulling of pulling segment 40 of electrosurgical slicing wire 34, in accordance with an application of the present invention. Although the configuration of FIG. 2B is illustrated in FIG. 2D, the techniques of FIG. 2D are equally applicable to the configuration of FIG. 2C.

For some applications, slicing segment 36 of electrosurgical slicing wire 34 is detachably embedded in wall 38 of retrieval bag 30, such as shown in FIG. 2B and FIG. 2C, as well as View A in FIG. 2D. For some of these applications:
slicing segment 36 of electrosurgical slicing wire 34 is detachably embedded in wall 38 of retrieval bag 30, such that wall 38 electrically insulates slicing segment 36 of electrosurgical slicing wire 34, and
tissue removal apparatus 20 is configured such that the pulling of pulling segment 40 of electrosurgical slicing wire 34, during the application of RF electrosurgical power between electrosurgical slicing wire 34 and return electrode 42, detaches the successive longitudinal portions of slicing segment 36 from wall 38 of retrieval bag 30, electrically exposes the successive longitudinal portions as they are detached from wall 38 of retrieval bag 30, and slices resected tissue specimen 22 within retrieval bag 30.

For example, View A of FIG. 2D shows the entirety of slicing segment 36 of electrosurgical slicing wire 34 detachably embedded in wall 38 of retrieval bag 30. View B of FIG. 2D shows electrosurgical slicing wire 34 after a first longitudinal portion 44A of slicing segment 36 has been detached from wall 38 and electrically exposed. View C of FIG. 2D shows electrosurgical slicing wire 34 after a second longitudinal portion 44B of slicing segment 36, successive to first longitudinal portion 44A, has been detached from wall 38 and electrically exposed, in addition to first longitudinal portion 44A. Additional successive portions of slicing segment 36 are detached from wall 38 and electrically exposed, typically until slicing segment 36 is entirely detached from wall 38 and entirely electrically exposed. Typically, at any given time, only the most-recently electrically exposed longitudinal portion of slicing segment 36 slices resected tissue specimen 22, while earlier-exposed longitudinal portions are withdrawn into control handle 120, such as into elongate insertion tip 130 and are no longer used to slice the specimen.

Alternatively, for some applications, electrically-insulated slicing segment 36 of electrosurgical slicing wire 34 is electrically insulated by insulation other than insulation of wall 38 of retrieval bag 30, such as an electrical insulation jacket or coating, or as described hereinbelow with reference to FIGS. 2E, 2F, 2G, and/or 8.

For some applications, pulling segment 40 is proximal, along electrosurgical slicing wire 34, to the electrically-insulated slicing segment of electrosurgical slicing wire 34. Tissue removal apparatus 20 is configured such that the pulling of pulling segment 40 of electrosurgical slicing wire 34 detaches, from wall 38 of retrieval bag 30, the successive longitudinal portions in a proximal-to-distal direction along slicing segment 36 of electrosurgical slicing wire 34, such as shown in FIG. 2D.

For some applications, wall 38 of retrieval bag 30, in addition to or instead of electrically insulating slicing segment 36 of electrosurgical slicing wire 34 when detachable embedded in wall 38, fixates slicing segment 36 to wall 38 at predetermined locations on the wall.

Reference is made to FIGS. 2E-G, which are schematic illustrations of respective configurations of certain elements of tissue removal apparatus 20, in accordance with respective applications of the present invention. In these configurations, slicing segment 36 of electrosurgical slicing wire 34 is electrically insulated by an electrically-insulating strip 45. Electrically-insulating strip 45 is permanently coupled to inner surface 48 of wall 38 of retrieval bag 30, so as to detachably couple slicing segment 36 to wall 38 of retrieval bag 30. Typically, electrically-insulating strip 45 comprises a polymer film 51.

Configurations 1-4 of FIGS. 2F and 2G show respective configurations 47 of the coupling of strip 45 to slicing segment 36. FIG. 2E shows the same configuration as shown in Configuration 1 of FIGS. 2F and 2G.

Tissue removal apparatus 20 is configured such that the pulling of pulling segment 40 of electrosurgical slicing wire 34 (a) detaches the successive longitudinal portions of slicing segment 36 from wall 38 of retrieval bag 30 by tearing electrically-insulating strip 45, (b) electrically exposes the successive longitudinal portions as they are detached from wall 38, and (c) slices the resected tissue specimen within retrieval bag 30.

Typically, the pulling of pulling segment 40 of electrosurgical slicing wire 34 detaches the successive longitudinal portions of slicing segment 36 from wall 38 of retrieval bag 30 by tearing electrically-insulating strip 45 using primarily the mechanical force applied by slicing segment 36 to strip 45, rather than heat generated by the RF electrosurgical power applied between electrosurgical slicing wire 34 and return electrode 42.

For some applications, electrically-insulating strip 45 is configured to provide tear propagation along the strip as the successive longitudinal portions of slicing segment 36 are detached from wall 38 of retrieval bag 30. Optionally, polymer film 51 is configured to provide the tear propagation preferentially in a direction parallel to the length of electrically-insulating strip 45. Alternatively, polymer film 51 is configured to provide the tear propagation equally in all directions.

For some applications, polymer film 51 comprises polypropylene or polyethylene terephthalate (PET). For some applications, polymer film 51 comprises a polymer selected from the group of polymers consisting of: biaxially oriented polypropylene (BOPP), a polyimide (e.g., poly (4,4′-oxydiphenylene pyromellitimide) (e.g., Kapton® (E.I. du Pont de Nemours and Company))), polypropylene carbonate (PPC), and biaxially oriented polyethylene terephthalate (BoPET).

For some applications, such as shown in Configurations 1 and 2 of FIG. 2F and FIG. 2G and shown and labeled in FIG. 2E, polymer film 51 is folded over and wrapped around slicing segment 36 such that two portions 59A and 59B of a first surface 57A of polymer film 51 are coupled to each other (such as by adhesion or welding). Slicing segment 36 is thus encapsulated by strip 45.

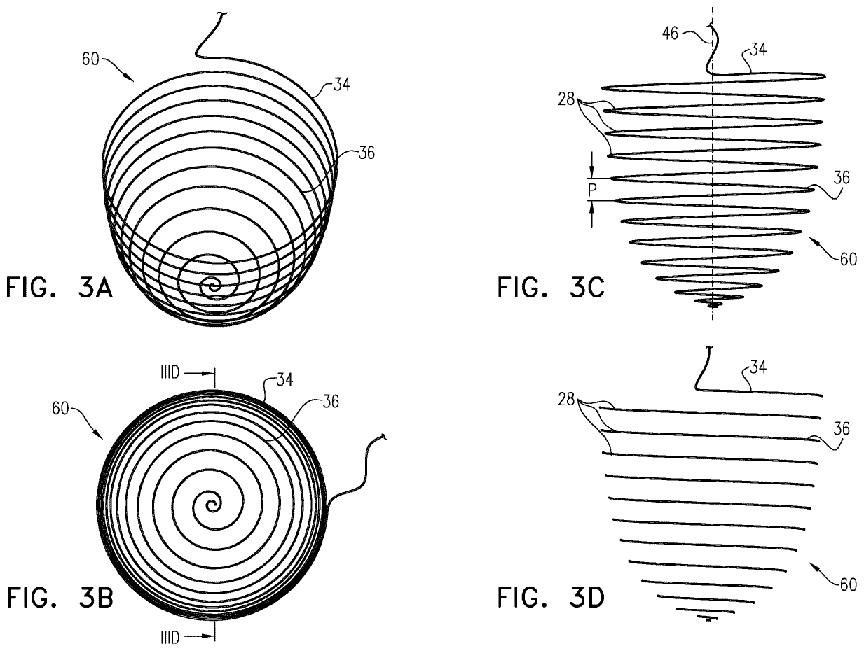

For some of these applications, such as shown in FIG. 3E and Configurations 1 and 2 of FIGS. 2F and 2G, a second surface 57B of polymer film 51, opposite first surface 57A, is permanently coupled to inner surface 48 of wall 38 of retrieval bag 30, so as to detachably couple slicing segment 36 to wall 38 of retrieval bag 30. For example, second surface 57B of polymer film 51 may be permanently coupled to inner surface 48 of wall 38 of retrieval bag 30 by polymer welding. Alternatively or additionally, second surface 57B of polymer film 51 is permanently coupled to inner surface 48 of wall 38 of retrieval bag 30 by an adhesive 61 (for example, adhesive 61 may be provided on one or more pieces of a double-sided adhesive tape (not shown)). Further alternatively, second surface 57B of polymer film 51 may be permanently coupled to inner surface 48 of wall 38 of retrieval bag 30 by a buffer layer 49, described hereinbelow with reference to FIG. 2E and Configurations 1-4 of FIG. 2F.

For some applications, electrically-insulating strip 45 comprises an adhesive 55 coated onto polymer film 51 (in which case, electrically-insulating strip 45 may be considered to comprise adhesive tape).

Typically, adhesive 55 is disposed on only first surface 57A of polymer film 51.

For some applications, adhesive 55 comprises an acrylic-based adhesive.

For some applications, such as shown in Configurations 1 and 2 of FIG. 2F and FIG. 2G and shown and labeled in FIG. 2E, polymer film 51 is folded over and wrapped around slicing segment 36 such that two portions 59A and 59B of first surface 57A are adhered to each other by adhesive 55. Slicing segment 36 is thus encapsulated by strip 45.

For other applications, such as shown in Configurations 3 and 4 in FIGS. 2F and 2G, electrically-insulating strip 45 is permanently coupled to inner surface 48 of wall 38 of retrieval bag 30 by adhesive 55. For some of these applications, such as shown in Configuration 3 in FIGS. 2F and 2G, slicing segment 36 is sandwiched between electrically-insulating strip 45 and inner surface 48 of wall 38 of retrieval bag 30. For others of these applications, slicing segment 36 is encapsulated by electrically-insulating strip 45 in a pod-like arrangement.

Reference is made to FIG. 2E and Configurations 1-4 of FIG. 2F. For some applications, tissue removal apparatus 20 further comprises buffer layer 49, which is coupled to inner surface 48 of wall 38 of retrieval bag 30 and electrically-insulating strip 45, so as to permanently couple electrically-insulating strip 45 to inner surface 48 of wall 38 of retrieval bag 30 via buffer layer 49. Buffer layer 49 may comprise a thermally insulating material, which is optionally also electrically insulating. For example, buffer layer 49 may comprise a polymer, such as, for example, a polyimide film (e.g., poly (4,4′-oxydiphenylene pyromellitimide) (e.g., Kapton® (E.I. du Pont de Nemours and Company)) or a polyester (e.g., polyethylene terephthalate (PET)). (In the configuration shown in FIG. 2E and Configurations 1 and 2 of FIG. 2F, buffer layer 49 permanently couples second surface 57B of polymer film 51 of strip 45 to inner surface of wall 38 of retrieval bag 30 via buffer layer 49. In Configurations 3 and 4 of FIG. 2F, buffer layer 49 permanently couples electrically-insulating strip 45 to inner surface 48 of wall 38 of retrieval bag 30, via buffer layer 49, by adhesive 55.)

For some applications, slicing segment 36 runs along a length of electrically-insulating strip 45. For some of these applications, a length of electrically-insulating strip 45 is at least 100 cm, such as at least 200 cm, e.g., at least 250 cm, and/or greater than or equal to a length of slicing segment 36.

Reference is made to FIG. 2E. For some applications, when retrieval bag 30 is in the expanded configuration, such as shown in FIG. 2E:
retrieval bag 30 defines a central longitudinal axis 46, and
electrically-insulating strip 45 and slicing segment 36 of electrosurgical slicing wire 34 wind around central longitudinal axis 46 at varying locations along central longitudinal axis 46.

Reference is made to FIGS. 1-2C and 2A-G.

For some applications, a length of slicing segment 36 is at least 100 cm, such as at least 200 cm, e.g., at least 250 cm.

For some applications, wall 38 of retrieval bag 30 comprises thermoplastic polyurethane (TPU), polyethylene, polypropylene, or, more generally, a co-extruded polymer compound film.

Reference is still made to FIGS. 1-2C and 2A-G. For some applications, tissue removal apparatus 20 is configured such that the pulling of pulling segment 40 of electrosurgical slicing wire 34, during application of RF electrosurgical power between electrosurgical slicing wire 34 and return electrode 42 when resected tissue specimen 22 is within retrieval bag 30 and return electrode 42 is in electrical contact with resected tissue specimen 22, slices resected tissue specimen 22 within retrieval bag 30:
into ten or fewer pieces 150 of tissue, such as five or fewer pieces 150, e.g., three or fewer pieces 150, such as a single piece 150 of tissue, such as shown and labeled in FIGS. 16F-I, described hereinbelow; and/or
into one or more pieces 150 of tissue, at least one of which is long, e.g., has a length greater than a multiple of a greatest thickness T of the piece 150 of tissue perpendicular to the length (at the location along the piece 150 having the greatest thickness T), the multiple equal to 10, such as equal to 20, such as shown and labeled in FIG. 16I, described hereinbelow.

The shape of the tissue may affect the number of pieces 150. Slicing resected tissue specimen 22 into a relatively small number of pieces, ideally a single long piece (i.e., a long strip) when possible, or a single long piece and a small number of smaller pieces, reduces the number of pieces that must be separately removed from retrieval bag 30 during the tissue removal procedure, such as described hereinbelow with reference to FIGS. 16F-I. This may substantially shorten the length of the tissue removal procedure. For example, tissue extraction from retrieval bag 30, after completion of the tissue slicing, may take less than 10 minutes, typically less than 2 minutes.

Reference is again made to FIGS. 1-2C, and is additionally made to FIGS. 3A-D, which are schematic illustrations of electrosurgical slicing wire 34, in accordance with an application of the present invention. For clarity of illustration, FIGS. 3A-D do not show retrieval bag 30 or other elements of tissue removal apparatus 20. FIG. 3D is a cross-sectional view. For some applications, when retrieval bag 30 is in the above-mentioned expanded configuration, such as shown in FIGS. 1-2D and 3A-D:
retrieval bag 30 defines central longitudinal axis 46 (labeled in FIGS. 2A, 2E, and 3C, even though retrieval bag 30 is not shown in FIG. 3C), and
slicing segment 36 of electrosurgical slicing wire 34 winds around central longitudinal axis 46 at varying locations along central longitudinal axis 46.

For some applications, slicing segment 36 of electrosurgical slicing wire 34 winds around central longitudinal axis 46 in at least 4 turns 28, such as in at least 6 turns 28, and/or no more than 100 turns 28, such as no more than 60 turns 28 (labeled in FIGS. 3C and 3D).

For some applications, opening 32 is at a proximal end 50 of retrieval bag 30 opposite a distal end 52 of retrieval bag 30 at a base 54 of retrieval bag 30 (labeled in FIG. 2A) (base 54 may be considered a distal apex of retrieval bag 30). Optionally, a distal end of slicing segment 36 is detachably coupled to wall 38 of retrieval bag 30 within a distance of 1 cm of a location 53 at which central longitudinal axis 46 intercepts base 54 (labeled in FIG. 2A), the distance measured along central longitudinal axis 46; for example, the distal end of slicing segment 36 may be detachably coupled to wall 38 of retrieval bag 30 at location 53.

For some applications, such as shown in FIGS. 1-2D and 3A-D, slicing segment 36 of electrosurgical slicing wire 34 winds around central longitudinal axis 46 in a base-to-opening direction at a nondecreasing distance from central longitudinal axis 46 when retrieval bag 30 is in the expanded configuration.

For some applications, such as shown in FIGS. 1-2D and 3A-D, slicing segment 36 of electrosurgical slicing wire 34 is shaped as a generally three-dimensional spiral 60 when retrieval bag 30 is in the expanded configuration.

For some applications, a pitch P (labeled in FIG. 3C) of the turns 28 of slicing segment 36 is at least 3 mm (e.g., at least 5 mm), no more than 20 mm (e.g., no more than 10 mm), and/or 3-20 mm, e.g., 5-10 mm, such as 7.5 mm. The pitch P may be constant along central longitudinal axis 46, or may vary along central longitudinal axis 46, e.g., increase in an opening-to-base direction, such as shown.

For some applications, a length of slicing segment 36 of electrosurgical slicing wire 34 equals at least 4 times and/or no more than 60 times a perimeter of opening 32 of retrieval bag 30.

Reference is again made to FIGS. 1-2D. For some applications, when retrieval bag 30 is in the above-mentioned expanded configuration, such as shown in FIGS. 1-2C (and FIG. 2D), and labeled in FIG. 2A:
opening 32 is at proximal end 50 of retrieval bag 30 opposite distal end 52 of retrieval bag 30 at base 54 of retrieval bag 30, and
(a) a first location 56A on central longitudinal axis 46 corresponding to opening 32 (or the perimeter of the bag defining the opening) of retrieval bag 30 (i.e., at the same height as opening 32 if central longitudinal axis 46 is oriented vertically, as shown in FIG. 2A) is at least a distance D of 5 cm from (b) a second location 56B on central longitudinal axis 46 corresponding to a proximal-most location 58 on wall 38 of retrieval bag 30 at which slicing segment 36 is detachably coupled to wall 38 (i.e., at the same height as proximal-most location 58 if central longitudinal axis 46 is oriented vertically), when retrieval bag 30 is in the expanded configuration.

Reference is made to FIG. 2B. For some applications, wall 38 of retrieval bag 30 comprises an inner electrical insulation layer 62 and an outer electrical insulation layer 64, disposed outward of inner electrical insulation layer 62 with respect to the interior of retrieval bag 30 (the interior is to the right of the enlargement in FIG. 2B, as well in FIG. 2C, described hereinbelow). Slicing segment 36 of electrosurgical slicing wire 34 is detachably embedded in wall 38 of retrieval bag 30 between inner electrical insulation layer 62 and outer electrical insulation layer 64. Tissue removal apparatus 20 is configured such that the pulling of pulling segment 40 of electrosurgical slicing wire 34, during the application of the RF electrosurgical power between electrosurgical slicing wire 34 and return electrode 42, pulls the successive longitudinal portions of slicing segment 36 of electrosurgical slicing wire 34 through inner electrical insulation layer 62 and detaches the successive longitudinal portions from outer electrical insulation layer 64, thereby electrically exposing the successive longitudinal portions, such as described hereinabove with reference to FIG. 2D.

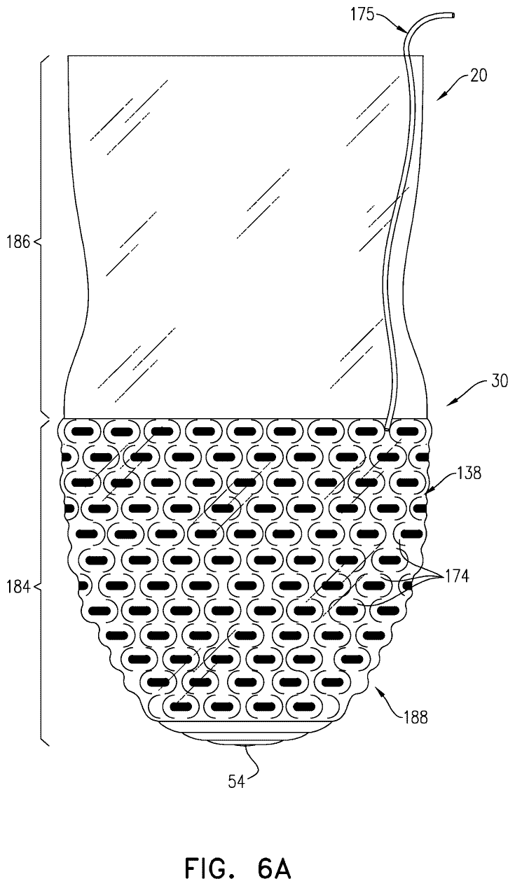

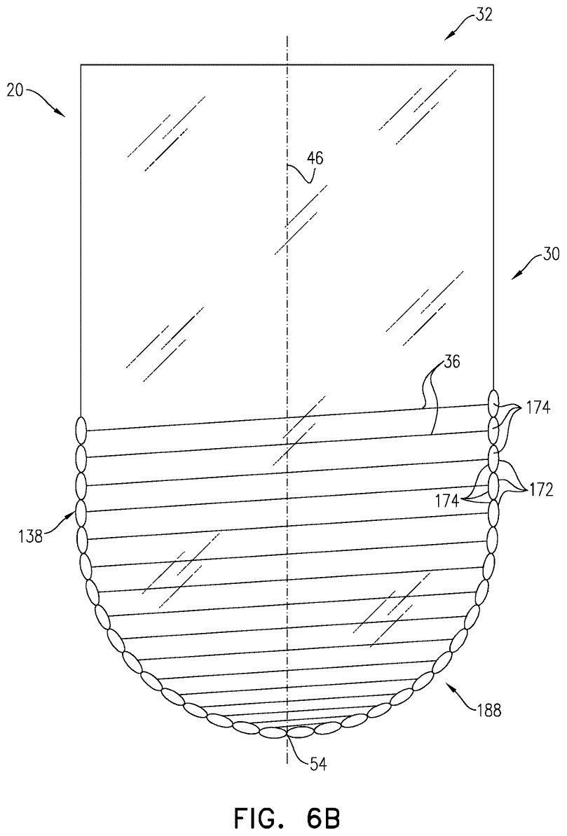

For some applications, inner electrical insulation layer 62 and outer electrical insulation layer 64 are adhered to each other, e.g., by welding and/or using an adhesive. Outer electrical insulation layer 64 is not necessarily the outer-most layer of wall 38 of retrieval bag 30; for example, wall 38 may comprise one or more additional layers and/or inflatable chambers, such as described hereinbelow with reference to FIGS. 6A-B, 7A-B, and/or 8.

Inner electrical insulation layer 62 and outer electrical insulation layer 64 may comprise the same material or different materials. Typically, both layers comprise polymers, which optionally may comprise additives for enhanced performance.

For some of these applications, a thickness of outer electrical insulation layer 64 is greater than a thickness of inner electrical insulation layer 62, such as at least 110% (e.g., at least 120%) of the thickness of inner electrical insulation layer 62.

Optionally, wall 38 of retrieval bag 30 has one or more of the following dimensions:
inner electrical insulation layer 62 has a thickness of at least 5 microns (e.g., at least 10 microns, such as at least 20 microns), no more than 40 microns (e.g., no more than 30 microns), and/or 5-40 microns, e.g., 10-40 microns, such as 20-40 microns or 20-30 microns, and/or
outer electrical insulation layer 64 has a thickness of at least 20 microns, no more than 250 microns (e.g., no more than 100 microns, such as no more than 50 microns), and/or 20-250 microns, e.g., 20-100 microns, e.g., 20-40 microns, such as 30-40 microns,

Reference is made to FIG. 2C. For some applications, slicing segment 36 of electrosurgical slicing wire 34 is detachably embedded in a single electrical insulation layer 66 of wall 38 of retrieval bag 30.

Reference is made to FIGS. 2B and 2C. Optionally, wall 38 of retrieval bag 30 is manufactured using vacuum forming (which may result in the bulges shown in FIG. 2B), or by injection molding (which may avoid bulging, as shown in FIG. 2C).

Reference is again made to FIGS. 1-2C, which show slicing segment 36 of electrosurgical slicing wire 34 embedded in wall 38 of retrieval bag 30 (and thus surrounded by wall 38). Thus, in this configuration, slicing segment 36 is not surrounded by electrical insulation distinct from wall 38 of retrieval bag 30 (such as an electrical insulation jacket or coating). Alternatively, slicing segment 36 is surrounded by electrical insulation distinct from, and coupled to, wall 38 of retrieval bag 30, such as an electrical insulation jacket or coating, in which case slicing segment 36 is not embedded in wall 38 of retrieval bag 30 (configuration not shown).

Reference is still made to FIGS. 1-2C. For some applications, wall 38 of retrieval bag 30 comprises one (such as shown in FIG. 2C) or more (such as shown in FIG. 2B) electrical insulation layers 68 that electrically insulate slicing segment 36 of electrosurgical slicing wire 34, and an electrically-conductive safety layer 70, disposed outward of the one or more electrical insulation layers 68. Retrieval bag 30 further comprises an electrical lead 72 that is electrically coupled to electrically-conductive safety layer 70 and terminates outside retrieval bag 30.

For some applications, electrically-conductive safety layer 70 comprises an at least partially electrically conductive polymer.

For some applications, tissue removal system 10 further comprises a voltage monitoring circuit 74. Although voltage monitoring circuit 74 is shown schematically in FIG. 2B, it may also be provided in the configurations shown in FIGS. 1, 2A, and 2C-D. Optionally, control handle 120, described hereinbelow with reference to FIGS. 15A-C, such as circuitry 123 thereof, comprises voltage monitoring circuit 74.

Electrical lead 72 is couplable to voltage monitoring circuit 74. Tissue removal system 10 is configured to cease the application of the RF electrosurgical power between electrosurgical slicing wire 34 and return electrode 42 upon sensing, by voltage monitoring circuit 74, that a voltage in electrically-conductive safety layer 70 exceeds a threshold level indicative of electrical leakage through the one or more electrical insulation layers of wall 38 of retrieval bag 30 to electrically-conductive safety layer 70.

Reference is still made to FIGS. 1-2C. In an application of the present invention, a tissue removal system 10 is provided that comprises a tissue removal apparatus 20 comprising:
retrieval bag 30 having opening 32 for receiving resected tissue specimen 22 within the patient's body 24;
return electrode 42; and
an electrosurgical electrode, which is configured, during application of RF electrosurgical power between the electrosurgical electrode and return electrode 42 when resected tissue specimen 22 is within retrieval bag 30 and return electrode 42 is in electrical contact with resected tissue specimen 22, to cut resected tissue specimen 22 within retrieval bag 30.

Retrieval bag 30 comprises wall 38, which comprises one or more electrical insulation layers 68, which electrically isolate at least a portion of the electrosurgical electrode from portions of the patient's body 24 outside retrieval bag 30, when retrieval bag 30 is disposed partially within the patient's body 24; and electrically-conductive safety layer 70, disposed outward of the one or more electrical insulation layers 68. Retrieval bag 30 further comprises electrical lead 72, which is electrically coupled to electrically-conductive safety layer 70 and terminates outside retrieval bag 30.

Optionally, the electrosurgical electrode comprises slicing segment 36 of electrosurgical slicing wire 34, as described above. Alternatively, the electrosurgical electrode comprises a different type of electrode, such as one or more blades.

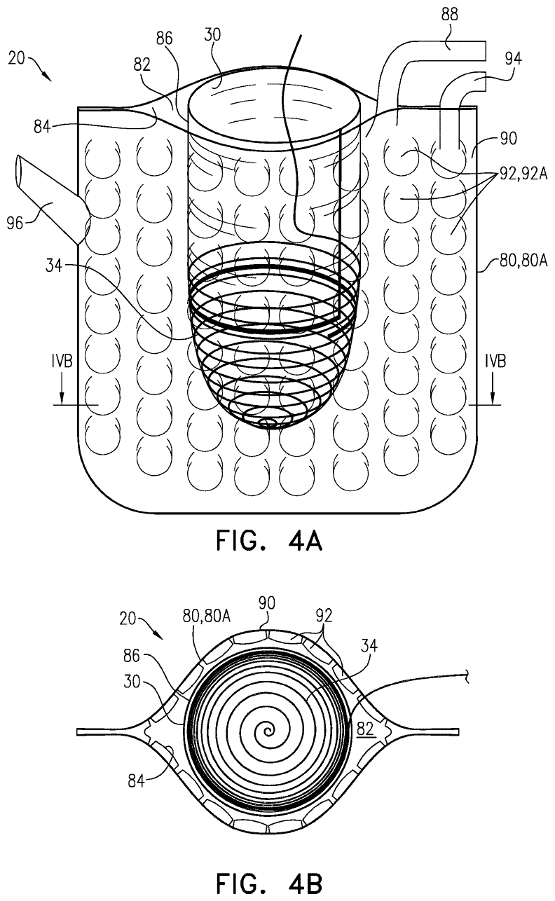

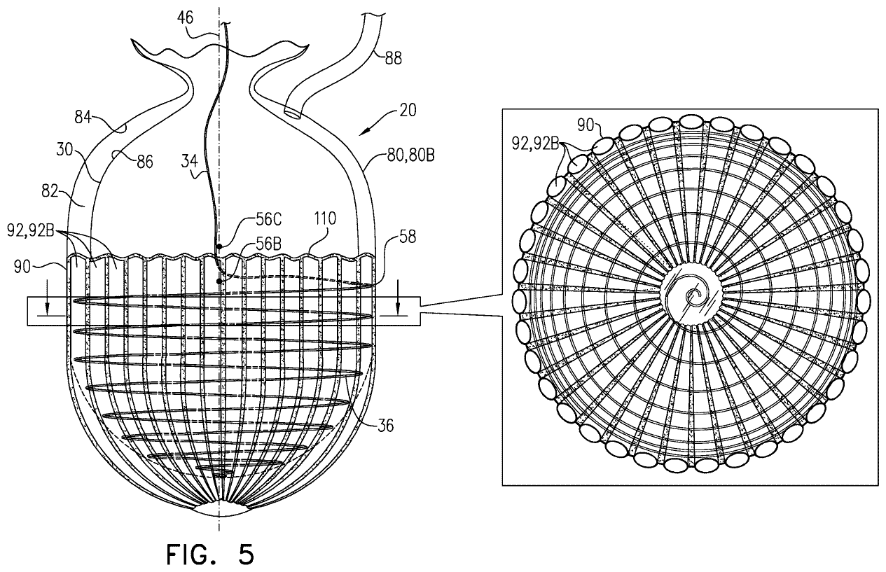

Reference is now made to FIGS. 4A-B and 5, which are schematic illustrations of alterative configurations of tissue removal apparatus 20 in which the apparatus further comprises a containment bag 80, in accordance with respective applications of the present invention. These configurations may be implemented in combination with any of the features of tissue removal system 10 described hereinabove and hereinbelow, including, but not limited to, return electrode 42A.

Retrieval bag 30 is disposed within containment bag 80, so as to define one or more inflatable chambers 82 (e.g., exactly one inflatable chamber 82, such as shown) between an inner surface 84 of containment bag 80 and an outer surface 86 of retrieval bag 30. (For clarity of illustration, the openings of retrieval bag 30 and containment bag 80 are shown as disconnected from each other in FIGS. 4A-B, while in actual practice they are connected (e.g., welded) together in order to close the one or more inflatable chambers 82, similar to the configuration shown in FIG. 5.) Containment bag 80 may help prevent the escape of cells of resected tissue specimen 22 to other areas of the patient's body 24, in the event of accidental puncture of retrieval bag 30. Containment bag 80 may also help prevent accidental cutting of surrounding tissue and/or organs by slicing segment 36 of electrosurgical slicing wire 34 in the event that the slicing segment accidentally punctures outer surface 86 of retrieval bag 30.

Typically, tissue removal apparatus 20 further comprises an inflatable-chambers inflation tube 88 in fluid communication with all of the one or more inflatable chambers 82 between inner surface 84 of containment bag 80 and outer surface 86 of retrieval bag 30.

Optionally, a portion of containment bag 80 is welded to retrieval bag 30, e.g., the openings of the containment and retrieval bags are welded together.

For some applications in which tissue removal apparatus 20 containment bag 80, tissue removal apparatus 20 does not comprise electrically-conductive safety layer 70, described hereinabove with reference to FIGS. 1-2C.

For some applications, a wall 90 of containment bag 80 is shaped so as to define a plurality of inflatable compartments 92 arranged entirely around containment bag 80. Upon inflation, inflatable compartments 92 may help push surrounding organs and tissue farther away from retrieval bag 30 and slicing segment 36 of electrosurgical slicing wire 34, thereby reducing the risk of damage to the surrounding anatomy. Typically, (a) a location 56C along central longitudinal axis 46 corresponding to a proximal edge 110 of the plurality of inflatable compartments 92 is at or proximal to (b) location 56B along central longitudinal axis 46 corresponding to proximal-most location 58 on wall 38 of retrieval bag 30 at which slicing segment 36 is detachably coupled to wall 38, when retrieval bag 30 is in the expanded configuration.

Typically, tissue removal apparatus 20 comprises an inflatable-compartments inflation tube 94 in fluid communication with all of inflatable compartments 92 (shown in FIGS. 4A-B, but also present in practice in the configuration of FIG. 5).

For some applications, wall 90 of containment bag 80 comprises inner and outer layers, which are coupled together (e.g., by welding and/or using an adhesive) along a plurality of lines and/or curves, so as to define inflatable compartments 92 between the inner and outer layers.

For some applications, containment bag 80 comprises a camera port 96 open to one of the one or more inflatable chambers 82. This configuration may be particularly useful in configuration in which wall 90 of containment bag 80 is not shaped so as to define inflatable compartments 92.

In the configuration of containment bag 80, 80A shown in FIGS. 4A-B, inflatable compartments 92, 92A are arranged in two-dimension array 98. In this configuration, wall 90 of containment bag 80, 80A may be shaped, for example, so as to define at least 10 inflatable compartments 92, 92A, no more than 500 inflatable compartments 92, 92A, and/or between 10 and 500 inflatable compartments 92, 92A.

In the configuration of containment bag 80, 80B shown in FIG. 5, inflatable compartments 92, 92B are arranged in a plurality of elongate strips oriented generally parallel to central longitudinal axis 46 of retrieval bag 30. In this configuration, wall 90 of containment bag 80, 80B may be shaped, for example, so as to define at least 10 inflatable compartments 92, 92A, no more than 500 inflatable compartments 92, 92A, and/or between 10 and 500 inflatable compartments 92, 92B.

Alternatively, on other applications of the present invention, wall 90 of containment bag 80 is not shaped so as to define any inflatable compartments, such as in the configurations shown in FIGS. 16B-I and 18, described hereinbelow.

Reference is still made to FIGS. 4A-B and 5. In an application of the present invention, a tissue removal system 10 is provided that comprises a tissue removal apparatus 20 comprising:
retrieval bag 30 having opening 32 for receiving resected tissue specimen 22 within the patient's body 24;
one or more tissue cutters detachably coupled to wall 38 of retrieval bag 30, which are configured to cut resected tissue specimen 22 when resected tissue specimen 22 is within retrieval bag 30; and
containment bag 80 within which retrieval bag 30 is disposed, so as to define one or more inflatable chambers 82 between inner surface 84 of containment bag 80 and outer surface 86 of retrieval bag 30.

For some applications, the one or more tissue cutters comprise one or more cutting wires, which may either not be electrically couplable to a generator or may be coupled electrically couplable to a generator, in which case tissue removal apparatus 20 typically further comprises one or more electrical leads for coupling the one or more tissue cutters to the generator. For example, the one or more tissue cutters may comprise slicing segment 36 of electrosurgical slicing wire 34, as described above. Alternatively, the one or more tissue cutters comprise a different type of tissue cutter, such as one or more blades.

Reference is now made to FIGS. 6A-B, which are schematic isometric and cross-sectional views of another configuration of retrieval bag 30, in accordance with an application of the present invention.

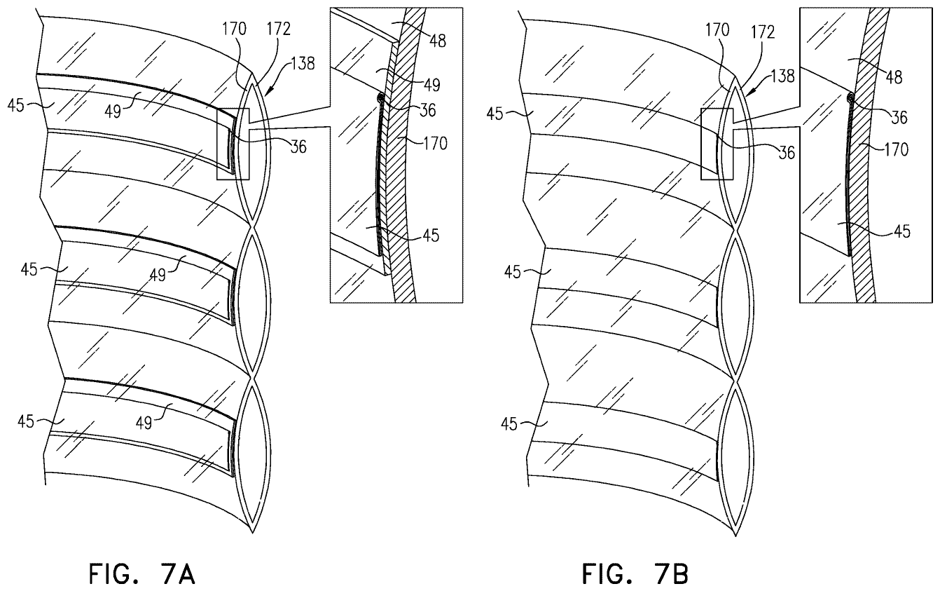

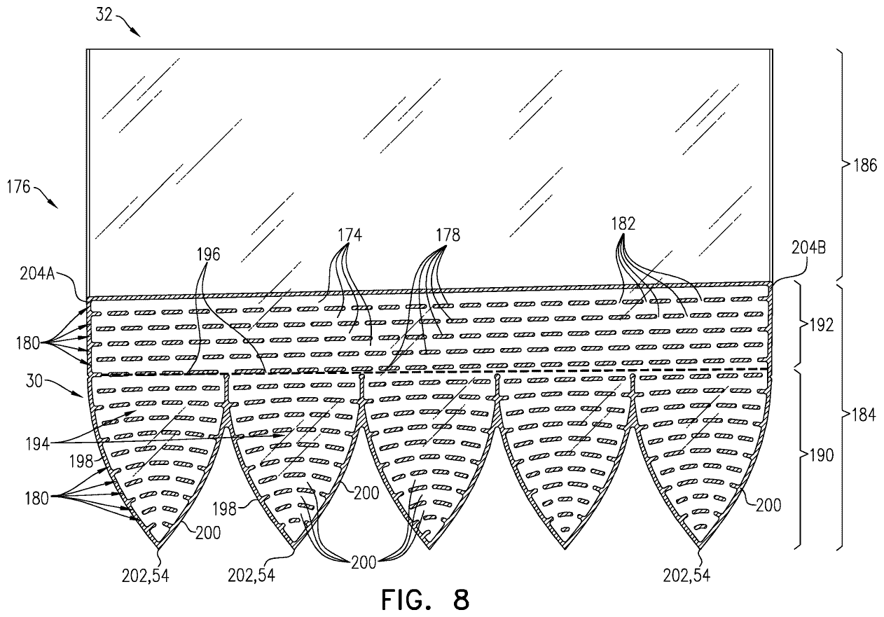

Reference is further made to FIGS. 7A-B, which are schematic illustrations of respective configurations of a wall 138 of retrieval bag 30 and certain other elements of tissue removal apparatus 20, in accordance with respective applications of the present invention. Wall 138 is shown partially in cross-section. The techniques described with reference to FIGS. 6A-B and 7A-B (and FIG. 8) may be implemented in any of the configurations of retrieval bag 30 described herein, mutatis mutandis, in which case wall 38 of retrieval bag 30 comprises wall 138 of retrieval bag.

In the configurations shown in FIGS. 6A-B and 7A-B, wall 138 of retrieval bag 30 (either an entirety of wall 138 or a portion of wall 138, as described hereinbelow with reference to FIG. 8) comprises an inner layer 170 and an outer layer 172, which define therebetween one or more inflatable chambers 174. Typically, inflatable chambers 174 are collectively arranged entirely around retrieval bag 30. Upon inflation, the one or more inflatable chambers 174 may help push surrounding organs and tissue farther away from retrieval bag 30 and slicing segment 36 of electrosurgical slicing wire 34, thereby reducing the risk of damage to the surrounding anatomy. In addition, if slicing segment 36 should inadvertently tear inner layer 170, the one or more inflatable chambers 174 and/or outer layer 172 may prevent contact between slicing segment 36 and the surrounding anatomy. In the configurations shown in FIGS. 6A-B and 7A-B, tissue removal apparatus 20 typically comprises an inflatable-chambers inflation tube 175 (shown in FIG. 6A) in fluid communication with all of the one or more inflatable chambers 174. Typically, the one or more inflatable chambers 174 are filled with air or a liquid such as saline, for example using a syringe coupled to the inflation tube.

For some applications, inner layer 170 and outer layer 172 are coupled together (e.g., by welding and/or using an adhesive) so as to define the one or more inflatable chambers 174 between the inner and outer layers. For example, inner layer 170 and outer layer 172 may be welded together at a plurality of welding locations so as to define the one or more inflatable chambers 174 therebetween, optionally using the techniques described hereinbelow with reference to FIG. 8.

For some applications, when retrieval bag 30 is in the expanded configuration, at least some (e.g., all) of the one or more inflatable chambers 174 wind around central longitudinal axis 46 at varying locations along central longitudinal axis 46 (labeled in FIG. 6B). For example, a plurality of (e.g., all of) inflatable chambers 174 may collectively wind around central longitudinal axis 46 at varying locations along central longitudinal axis 46 (labeled in FIG. 6B), e.g., may be collectively shaped as a generally three-dimensional spiral.

Reference is made to FIGS. 7A-B. As described hereinabove with reference to FIGS. 2E-G, for some applications, when retrieval bag 30 is in the expanded configuration, electrically-insulating strip 45 and slicing segment 36 of electrosurgical slicing wire 34 wind around central longitudinal axis 46 at varying locations along central longitudinal axis 46. For some of these applications, when retrieval bag 30 is in the expanded configuration:
the one or more inflatable chambers 174 wind around central longitudinal axis 46 at varying locations along central longitudinal axis 46 (labeled in FIG. 6B), and
at least a portion of electrically-insulating strip 45 is permanently coupled to inner surface 48 of wall 138 of retrieval bag 30 along the one or more inflatable chambers 174 that wind around the central longitudinal axis, so as to detachably couple slicing segment 36 to wall 138 of retrieval bag 30 along the one or more inflatable chambers 174 that wind around the central longitudinal axis.

Optionally, such as shown in FIG. 7A and described hereinabove with reference to FIGS. 2E and 2F, buffer layer 49, which is coupled to inner surface 48 of wall 138 of retrieval bag 30 and electrically-insulating strip 45, so as to permanently couple electrically-insulating strip 45 to inner surface 48 of wall 138 of retrieval bag 30 via buffer layer 49.

Reference is now made to FIG. 8, which is a schematic illustration of a welding pattern 176 applied to retrieval bag 30, in accordance with an application of the present invention. FIG. 8 shows retrieval bag 30 cut open and laid flat, as described in more detail hereinbelow. As described hereinabove with reference to FIGS. 6A-B and 7A-B, inner layer 170 and outer layer 172 may be welded together at a plurality of welding locations so as to define the one or more inflatable chambers 174 therebetween. FIG. 8 shows one exemplary welding pattern 176 defining a plurality of welding locations 178. This exemplary welding pattern 176 defines a plurality of inflatable chambers 174 that collectively wind around central longitudinal axis 46 at varying locations along central longitudinal axis 46 (labeled in FIG. 6B), e.g., that are collectively shaped as a generally three-dimensional spiral. For some applications, the plurality of inflatable chambers 174 wind around central longitudinal axis 46 in a base-to-opening direction at a nondecreasing distance from central longitudinal axis 46 when retrieval bag 30 is in the expanded configuration; for example, the plurality of inflatable chambers 174 may collectively be shaped as a generally three-dimensional spiral when retrieval bag 30 is in the expanded configuration.

The plurality of inflatable chambers 174 are shaped so as to collectively define a plurality of turns 180, e.g., 5-30 turns, such as 14-22 turns. Optionally, adjacent turns 180 are in fluid communication with each other by a plurality of openings 182, which may allow the inflation fluid to reach all portions of inflatable chambers 174.

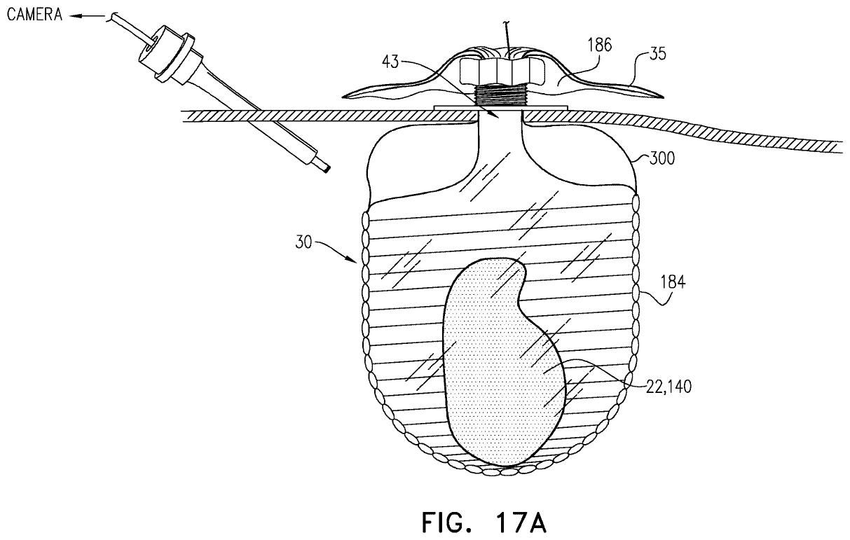

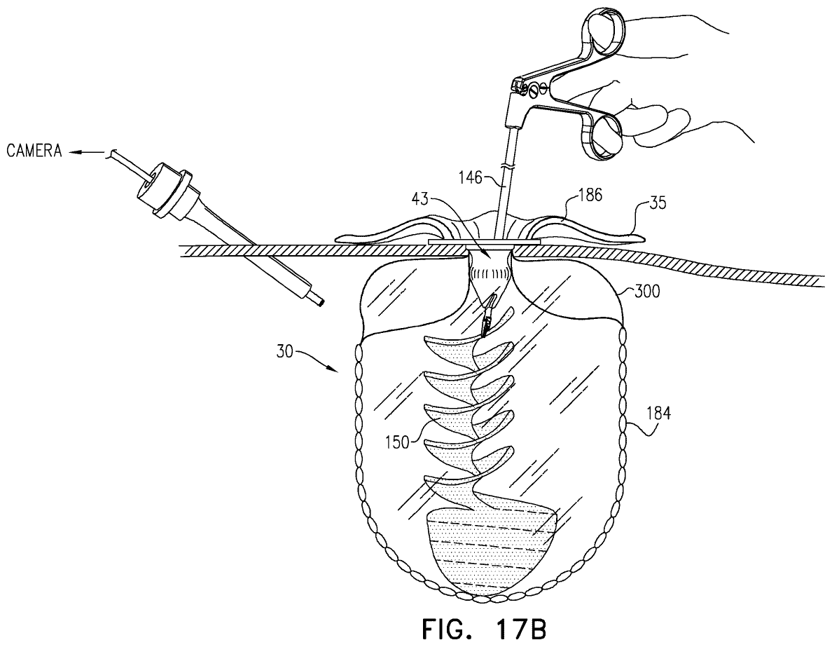

Typically, only an inflatable portion 184 of wall 138 of retrieval bag 30 defines the one or more inflatable chambers 174; inflatable portion 184 of wall 138 is inflated while within a body cavity, such as an abdomen 142, such as shown in FIGS. 17A-B, described hereinbelow. Wall 138 of retrieval bag 30 also typically includes a non-inflatable portion 186, which is disposed partially outside the patient's body, also as shown in FIGS. 17A-B. Non-inflatable portion 186 of wall 138 may comprise inner layer 170 and outer layer 172, or may comprise a single layer.

Reference is still made to FIG. 8. In some applications of the present invention, retrieval bag 30 is shaped so as to define a base portion 188 opposite opening 32 of retrieval bag 30. Wall 138 includes (a) a first portion 190 that defines base portion 188, and (b) a second portion 192 that is closer to opening 32 than first portion 190 is to opening 32.

For some applications, first portion 190 of wall 138 is defined by three or more triangles 194, e.g., five triangles (such as shown), six triangles, or seven or more triangles. Triangles 194:
have respective first base edges 196 that are integral with second portion 192 of wall 138,
have respective second curved edges 198 and third curved edges 200 that meet at respective apexes 202, and
comprise respective portions of inner layer 170 and outer layer 172, which define therebetween respective portions of the one or more inflatable chambers 174.

During manufacture, second curved edges 198 are fixed to (e.g., welded to) third curved edges 200 of adjacent respective ones of triangles 194, such that triangles 194 together define base portion 188 and the apexes together define base 54 of base portion 188 (base 54 is the bottom-most central point of base portion 188).

Because of the curvature of first and second curved edges 198 and 200, base portion 188 is not conical, but instead shaped as a paraboloid and/or a bowl.

In addition, during manufacture, opposite vertical edges 204A and 204B of second portion 192 of wall 138 are fixed to (e.g., welded to) each other, in order to form the portion of retrieval bag 30 defined by second portion 192 into a cylinder.

As mentioned above and shown in FIG. 8, adjacent turns 180 are in fluid communication with each other by the plurality of openings 182, which may allow the inflation fluid to reach all portions of inflatable chambers 174. It is noted that the fixation (e.g., welding) of second curved edges 198 to third curved edges 200 of adjacent respective ones of triangles 194, and/or the fixation (e.g., welding) of opposite vertical edges 204A and 204B of second portion 192 of wall 138 to each other, may locally interrupt fluid communication between inflatable chambers 174. Nevertheless, these local interruptions in fluid flow do not disturb the collective winding of the one or more inflatable chambers 174 around central longitudinal axis 46, including in first portion 190 of wall 138 that is defined by triangles 194. In other words, the path collectively defined by the plurality of inflatable chambers 174 winds around central longitudinal axis 46, e.g., the path may define a generally three-dimensional spiral. (Electrically-insulating strip 45, if provided, typically passes over the above-described local interruptions.)

As mentioned above, FIG. 8 shows retrieval bag 30 cut open and laid flat. As a result of the generally three-dimensional spiral shape of the plurality of inflatable chambers 174, in this laid-flat view inflatable chambers 174 are slightly inclined in the rightward direction. When opposite vertical edges 204A and 204B of second portion 192 of wall 138 are fixed to (e.g., welded to) each other, the left end of each turn of inflatable chambers 174 (at left vertical edge 204A) is joined with the right end of the distally adjacent turn of inflatable chambers 174 (at right vertical edge 204B).

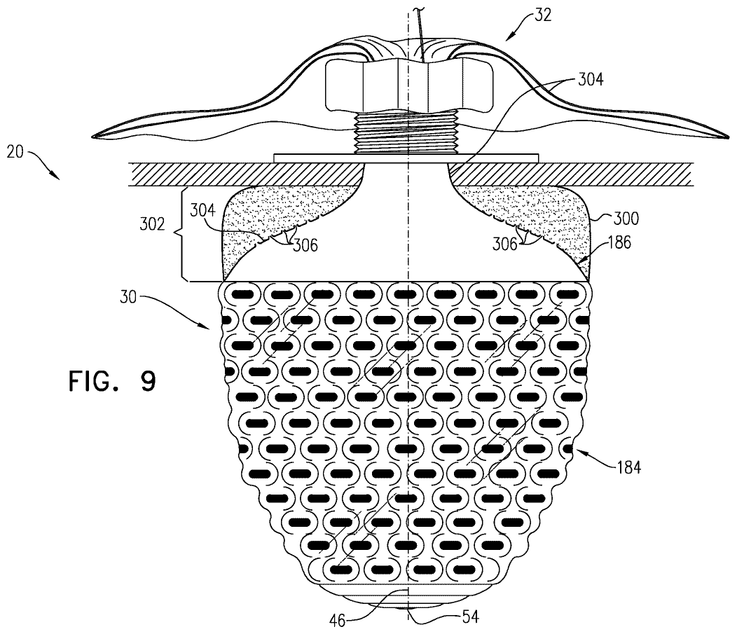

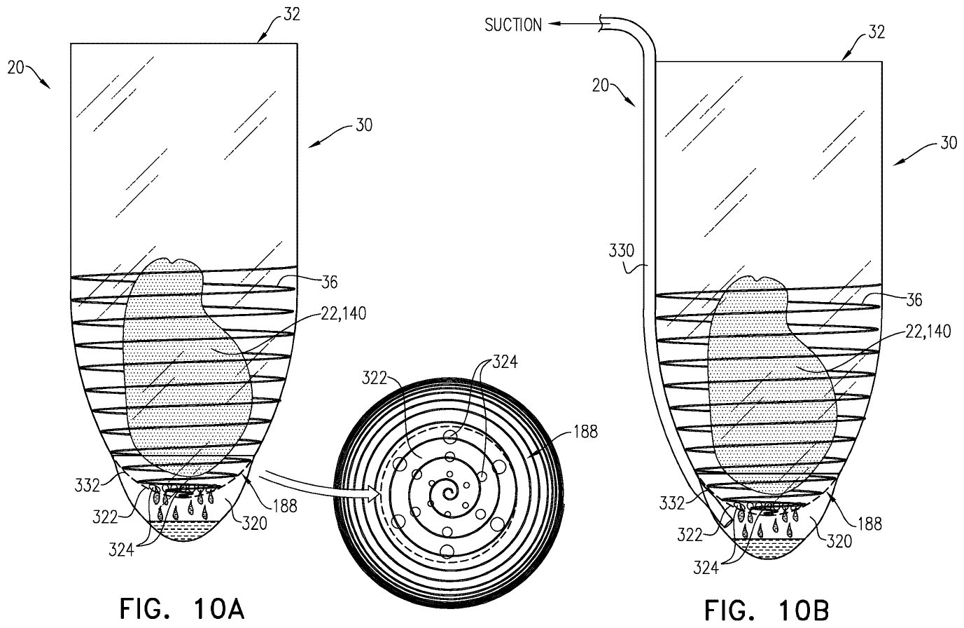

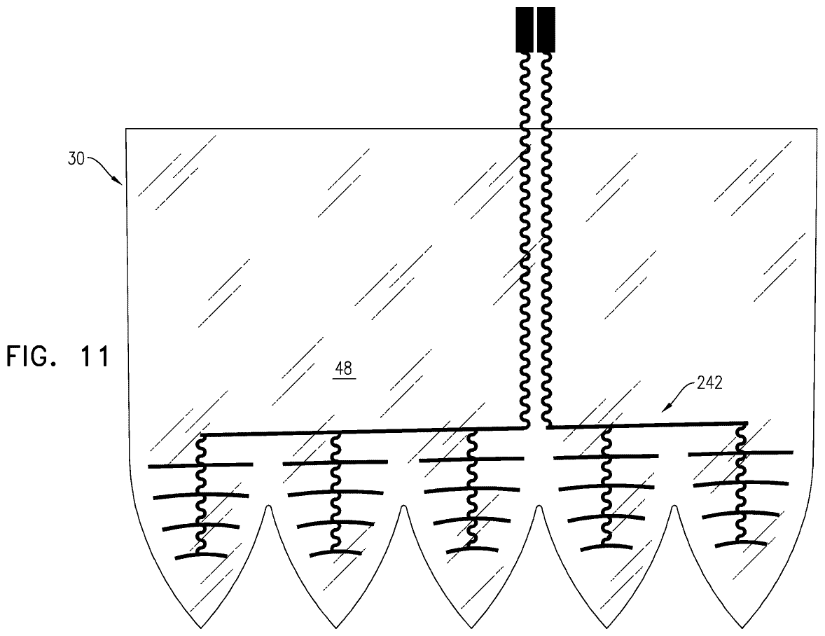

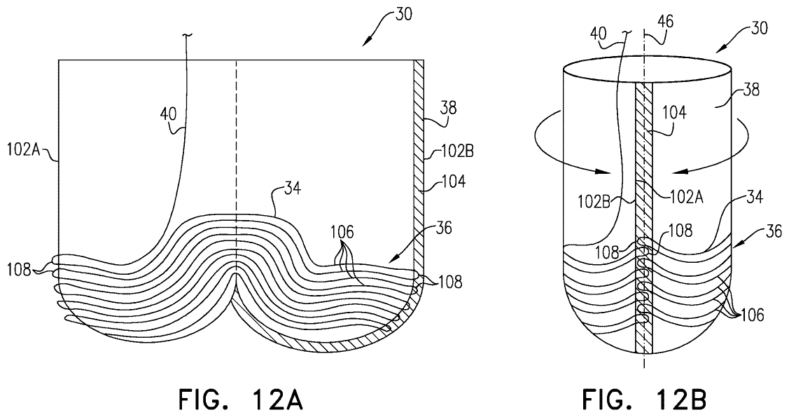

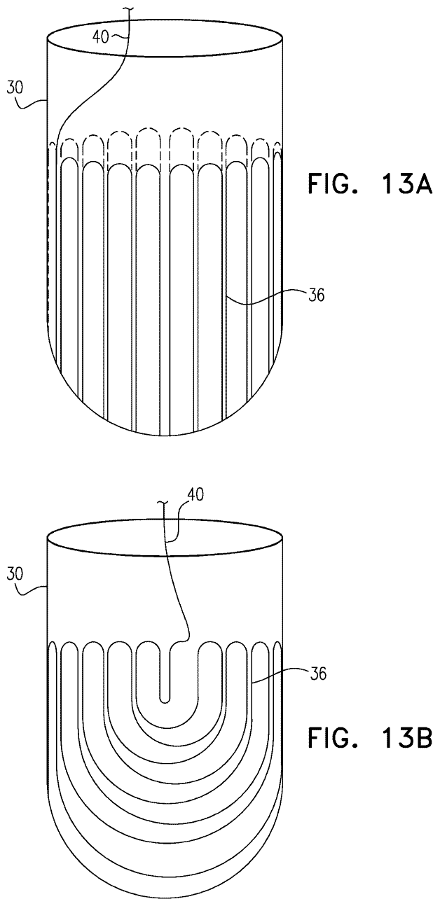

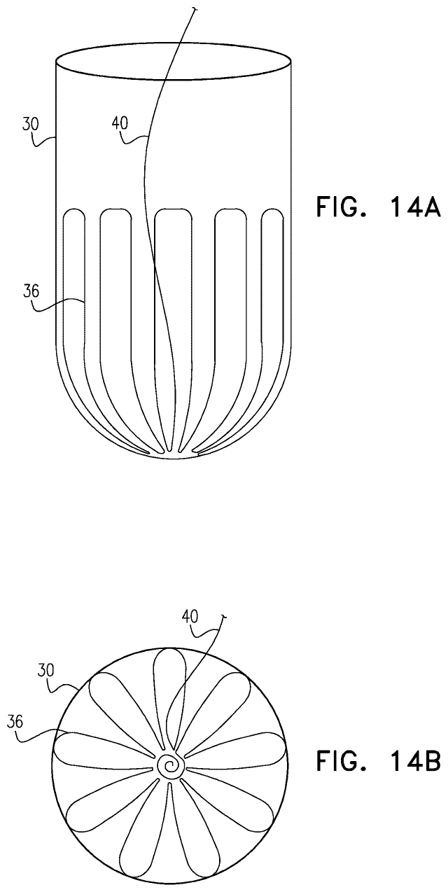

Reference is now made to FIG. 9, which is a schematic illustration of an alternative configuration of tissue removal apparatus 20, in accordance with an application of the present invention. This alternative configuration may be implemented in combination with any of the other configurations of tissue removal apparatus 20 described herein.

In this configuration, tissue removal apparatus 20 further comprises an annular inflatable positioning chamber 300, which is fixed to and encircles an axial portion 302 of an external surface 304 of retrieval bag 30, axial portion 302 axially between opening 32 and base 54, spaced away from opening 32 and from base 54. Annular inflatable positioning chamber 300 typically sets the location of retrieval bag 30 with respect to a laparoscopic port 43 (an opening in the abdominal wall), by allowing only a proximal portion 35 of retrieval bag 30 proximal to annular inflatable positioning chamber 300 to pass through laparoscopic port 43 as retrieval bag 30 is pulled out of the body through the laparoscopic port, such as described hereinbelow with reference to FIG. 16E.

For some applications, annular inflatable positioning chamber 300 is in fluid communication with an interior of retrieval bag 30, such as by one or more openings 306. Inflation of the interior of retrieval bag 30 thus also inflates annular inflatable positioning chamber 300. Alternatively, annular inflatable positioning chamber 300 is fluidly isolated from the interior of retrieval bag 30, and is inflated separately from the interior of retrieval bag 30.

For some applications, when retrieval bag 30 is in the expanded configuration and annular inflatable positioning chamber 300 is fully inflated, a greatest external diameter of annular inflatable positioning chamber 300 equals 100%-200% of a greatest external diameter of retrieval bag 30, such as 100%-150%, e.g., 100%-125% of the greatest external diameter of retrieval bag 30.

For some applications, when retrieval bag 30 is in the expanded configuration, axial portion 302 of external surface 304 of retrieval bag 30 is generally conical.

Alternatively or additionally, for some applications, when retrieval bag 30 is in the expanded configuration and annular inflatable positioning chamber 300 is fully inflated, an external surface of annular inflatable positioning chamber 300 is generally cylindrical.

For some applications, external surface 304 of axial portion 302 of retrieval bag 30 serves as an internal wall of annular inflatable positioning chamber 300.

For some applications, axial portion 302 of external surface 304 of retrieval bag 30 has a length, measured along central longitudinal axis 46, of 5-100 mm, such as 15-40 mm.

For some applications, such as described hereinabove with reference to FIGS. 6A-B and 7A-B, the wall of retrieval bag 30 comprises inner layer 170 and outer layer 172, which define therebetween the one or more inflatable chambers 174. For some of these applications, axial portion 302 of external surface 304 of retrieval bag 30 is a portion of non-inflatable portion 186 of wall 138, described hereinabove with reference to FIG. 8.

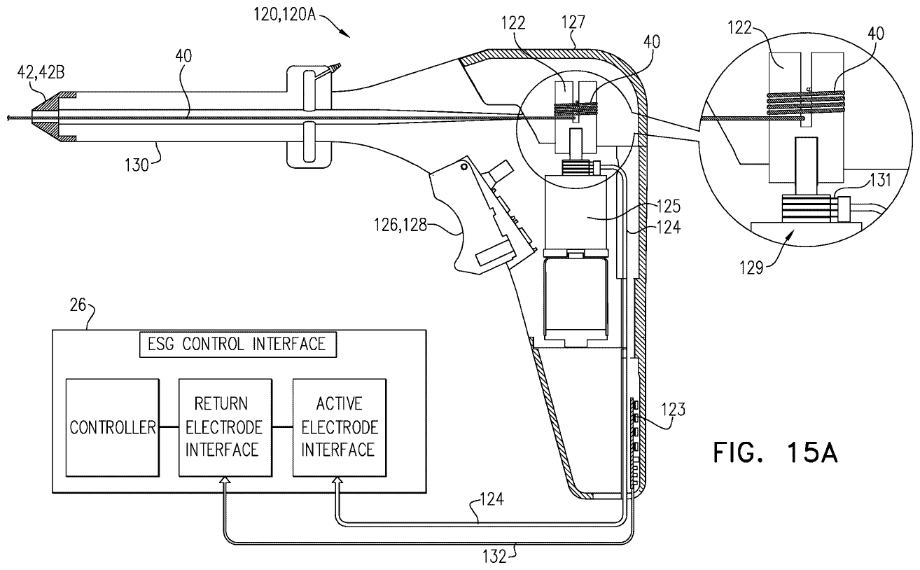

Reference is now made to FIGS. 10A-B, which are schematic illustrations of alternative configurations of tissue removal apparatus 20, in accordance with respective applications of the present invention. These alternative configurations may be implemented in combination with any of the other configurations of tissue removal apparatus 20 described herein.

In this configuration, opening 32 of retrieval bag 30 is a proximal opening 32, and base portion 188 of retrieval bag 30 is a distal base portion 188. Tissue removal apparatus 20 further comprises a drainage chamber 320 disposed distal to distal base portion 188. A wall 322 of distal base portion 188 of retrieval bag 30 is shaped so as to define one or more drainage openings 324 between the interior of retrieval bag 30 and drainage chamber 320. Providing drainage chamber 320 allows blood and other liquids to drain from the interior of retrieval bag 30 during a procedure. Excessive liquid within the interior of retrieval bag 30 may make the electrical cutting process more difficult. It is noted that retrieval bag 30 is typically oriented such that distal base portion 188 is below proximal opening 32, with respect to the Earth.

For some applications, such as shown in FIG. 10B, tissue removal apparatus 20 further comprises a tube 330 coupled in fluid communication with drainage chamber 320. Suction may be applied to tube 330 to drain drainage chamber 320 if necessary during the procedure.

For some applications, an external surface 332 of at least a portion of wall 322 of distal base portion 188 of retrieval bag 30 serves as an internal wall of drainage chamber 320.

For some applications, when retrieval bag 30 is in the expanded configuration, a volume of drainage chamber 320 equals (a) 5-50 ml, e.g., 10-30 ml, and/or (b) 0.5%-5% of a volume of retrieval bag 30, e.g., .75%-2% of the volume of retrieval bag 30.

Reference is now made to FIG. 11, which is a schematic illustration of an alternative configuration of tissue removal apparatus 20, in accordance with an application of the present invention. Retrieval bag 30 is shown cut and laid flat in FIG. 11. In this configuration, tissue removal apparatus 20 comprises a return electrode 242 formed on inner surface 48 by conductive ink printing. Return electrode 242 may optionally implement any of the features of return electrode 42, described hereinabove and/or hereinbelow.

Reference is now made to FIGS. 12A-B, which are schematic illustrations of an alternative arrangement of slicing segment 36 of electrosurgical slicing wire 34 detachably coupled to wall 38 of retrieval bag 30, in accordance with an application of the present invention. FIG. 12A shows inner surface 48 of retrieval bag 30 in a flatted configuration prior to formation into the shape of retrieval bag 30. FIG. 12B shows retrieval bag 30 after a first edge portion 102A of retrieval bag 30 has been coupled to a second edge portion 102B of retrieval bag 30, such as by an adhesive strip or polymer welding at a welding area 104 (for clarity of illustration, electrosurgical slicing wire 34 is shown with a solid line, even though the wire is actually inside and retrieval bag 30 and obscured by wall 38 of retrieval bag 30).

In this configuration, slicing segment 36 of electrosurgical slicing wire 34 is detachably coupled to wall 38 of retrieval bag 30 such that slicing segment 36 travels around central longitudinal axis 46 in at least four turns 106 in alternating clockwise and counterclockwise directions in axially sequential turns 106 when retrieval bag 30 is in the expanded configuration. This arrangement of slicing wire 34 might be considered a back-and-forth half-spiral.

Typically, successive turns 106 of slicing segment 36 along central longitudinal axis 46 are connected by respective end curved portions 108 of slicing segment 36. Typically, upon assembly of retrieval bag by coupling first edge portion 102A to second edge portion 102B of retrieval bag 30, end curved portions 108 are interdigitated, without contacting one another, to provide continuous electrosurgical coverage entirely around the circumference of retrieval bag 30.

Reference is now made to FIGS. 13A-B, which are schematic illustrations of another alternative arrangement of slicing segment 36 of electrosurgical slicing wire 34 detachably coupled to wall 38 of retrieval bag 30, in accordance with an application of the present invention.

Reference is now made to FIGS. 14A-B, which are schematic illustrations of yet another alternative arrangement of slicing segment 36 of electrosurgical slicing wire 34 detachably coupled to wall 38 of retrieval bag 30, in accordance with an application of the present invention.

Reference is now made to FIGS. 15A and 15B-C, which are schematic illustrations of a control handle 120, in accordance with respective applications of the present invention. In some applications of the present invention, tissue removal system 10 further comprises control handle 120, or another control handle providing similar functionality. FIGS. 15A-C also show RF ESG 26, which is typically not an element of tissue removal system 10, although it may optionally be an element thereof. RF ESG 26 may comprise a conventional RF ESG, which are known in the art and commercially available, configured with appropriate parameters for use as described herein.

Typically, pulling segment 40 of electrosurgical slicing wire 34 is electrically and mechanically couplable to control handle 120, typically by a healthcare worker during preparation for a procedure performed using tissue removal system 10. For example, control handle 120 may comprise a bobbin 122, and pulling segment 40 may be couplable to control handle 120 by partially wrapping pulling segment 40 around the bobbin. Bobbin 122 may comprise a conductive material, and may be electrically coupled to an electrical lead 124 (such as by one or more slip rings 129, e.g., comprising one or more brush contacts 131) that is couplable to RF ESG 26. Optionally, control handle 120 may comprise an openable or removable cover 127 that provides access to bobbin 122 for insertion of the wire, and then shields bobbin 122 from accidental contact during use (for example, the control handle may be configured to require that cover 127 be closed in order to activate the system, as a safety feature).

Reference is still made to FIGS. 15A and 15B-C. For some applications, control handle 120 comprises circuitry 123 and a motor 125 arranged to pull pulling segment 40 of electrosurgical slicing wire 34 during the application of RF electrosurgical power between electrosurgical slicing wire 34 and return electrode 42 or return electrode 242, when pulling segment 40 of electrosurgical slicing wire 34 is electrically and mechanically coupled to control handle 120. For example, motor 125 may be arranged to rotate bobbin 122 in configurations in which control handle 120 comprises bobbin 122. Optionally, circuitry 123 is configured to drive motor 125 to pull pulling segment 40 of electrosurgical slicing wire 34 with a predefined pulling force limit.

Reference is still made to FIGS. 15A and 15B-C. Typically, control handle 120 comprises a user control 126, such as, for example, a trigger 128, as shown. For some applications, user control 126 (e.g., trigger 128) activates control handle 120 in two steps: (1) a first step, in which motor 125 is activated (e.g., by circuitry 123) to pull pulling segment 40 of electrosurgical slicing wire 34 without applying the RF electrosurgical power to electrosurgical slicing wire 34, and (2) a second step, in which, in addition to activation of motor 125, RF electrosurgical power is applied to pulling segment 40, such as by activating RF ESG 26 (e.g., by circuitry 123). This two-step activation may apply sufficient tension to pulling segment 40 of electrosurgical slicing wire 34 before commencement of RF application. For example, partial squeezing of trigger 128 may activate the first step, and full squeezing of trigger 128 may activate the second step.

For some applications, during the first step, motor 125 is activated (e.g., by circuitry 123) to pull pulling segment 40 of electrosurgical slicing wire 34 without applying the RF electrosurgical power to electrosurgical slicing wire 34, until a longitudinal portion of slicing segment 36 of electrosurgical slicing wire 34 comes detached from wall 38 of retrieval bag 30. This detachment, using mechanical force only, without application of the RF electrosurgical power, may be possible because of the low mechanical strength of inner electrical insulation layer 62 (in the configuration of FIG. 2B) or of single electrical insulation layer 66 (in the configuration of FIG. 2C). For example, this mechanical-only detachment of a longitudinal portion of slicing segment 36 may be performed to adjust the starting point of slicing segment 36 on wall 38 to be no higher than the height of resected tissue specimen 22 within retrieval bag 30, such as in cases in which the resected tissue specimen is shorter than the portion of retrieval bag 30 to which slicing segment 36 is originally detachably coupled.

Reference is still made to FIGS. 15A and 15B-C. For some applications, control handle 120 is shaped so as to define an elongate insertion tip 130, which is shaped for insertion into the patient's body 24 and into retrieval bag 30 via opening 32, such as described hereinbelow with reference to FIG. 16F. Elongate insertion tip 130 is typically shaped so as to define a channel therethrough for insertion of pulling segment 40 of electrosurgical slicing wire 34.

Reference is still made to FIGS. 15A and 15B-C. For some applications, return electrode 42 or return electrode 242 is a return electrode 42B, and control handle 120 comprises return electrode 42, 42B, which may be disposed, for example, and the end of insertion tip 130. Insertion of insertion tip 130 into retrieval bag 30 brings return electrode 42, 42B into electrical contact with resected tissue specimen 22, typically direct physical contact with resected tissue specimen 22, such as shown in FIG. 16F. In these applications, control handle 120 typically comprises an electrical lead 132 which is electrically coupled to return electrode 42, 42B and electrically couplable to RF ESG 26 (for clarity of illustration, electrode lead 132 is not shown coupled to return electrode 42, 42B, although it is coupled thereto in practice).

For some applications, tissue removal apparatus 20 comprises both (a) return electrode 42A, described hereinabove with reference to FIGS. 2A-D, or return electrode 242, described hereinabove with reference to FIG. 11, and (b) return electrode 42B, described immediately above. Return electrodes 42A and 42B are electrically coupled to each other and electrically couplable to the return electrode receptacle of RF ESG 26. Providing both types of return electrode may provide better electrical contact with resected tissue specimen 22.

For some applications, control handle 120 is coupled to a smoke evacuation system, as are known in the art and commercially available.

Reference is made to FIGS. 15B-C. In some applications, control handle 120 further comprises a pump 244, which is in fluid communication with a channel 246 defined within elongate insertion tip 130 of control handle 120. Pump 244 is configured to deliver a fluid to inflate the interior of retrieval bag 30. Inflation of the interior of retrieval bag 30 may provide mechanical support to wall 38 or 138 of retrieval bag 30 to assist with pulling slicing segment 36 of electrosurgical slicing wire 34 from the wall of the retrieval bag. Typically, the interior of retrieval bag 30 is inflated to a pressure greater than an insufflation pressure to which the anatomical body cavity is insufflated. (It is noted that in configurations in which wall 138 of retrieval bag 30 is shaped so as to define the one or more inflatable chambers 174, the interior of retrieval bag 30 is typically not in fluid communication with the one or more inflatable chambers 174, and thus inflated separately.)

Reference is again made to FIGS. 15A and 15B-C. For some applications, control handle 120 implements one or more of the following control and monitoring features:
control handle 120 comprises a retrieval bag pressure sensor, which is configured to measure a pressure within the interior of retrieval bag 30; optionally, in configurations in which control handle 120 further comprises a pump 244, such as described hereinabove with reference to FIGS. 15B-C, control handle 120 is configured to activate pump 244 based on the measured pressure in order to control the inflation pressure of the bag;
control handle 120 comprises a pressure relief valve, which automatically allows the release of excess pressure from the interior of retrieval bag 30 in the event of inadvertent over-inflation; and/or
control handle 120 comprises a motor monitoring current sensing and encoder, and control handle 120 is configured to perform process monitoring at least partially based on the output of the motor monitoring current sensing and encoder.

Reference is made to FIGS. 16A-J, which are schematic illustrations of a method for removing a resected tissue specimen 22 from a body 24 of a patient, in accordance with an application of the present invention. Although the method is illustrated using tissue removal system 10, as described hereinabove with reference to FIGS. 1-3D and 15A-C, the method may also be practiced using other configurations of tissue removal system 10 described herein, mutatis mutandis, or other tissue removal systems suitable for performing the method, mutatis mutandis.

By way of example and not limitation, the method is illustrated for performing a hysterectomy (i.e., resected tissue specimen 22 is a uterus 140). The hysterectomy is performed in a minimally invasive procedure, either a laparoscopic hysterectomy, as shown, or a vaginal hysterectomy (approach not shown, but well known in the hysterectomy art). For the sake of brevity, conventional aspects of the hysterectomy are not described.

As shown in FIG. 16A, retrieval bag 30 of tissue removal apparatus 20 is inserted into abdomen 142 of the patient's body 24 via laparoscopic port 43, typically using a trocar 144, typically while retrieval bag 30 is in its compressed configuration, such as shown. For vaginal procedures, a vaginal port is used instead of a laparoscopic port.

As shown in FIG. 16B, tissue specimen 22 is resected, using techniques known in the art.

As shown in FIG. 16C, retrieval bag 30 is transitioned from its compressed configuration to its expanded configuration.

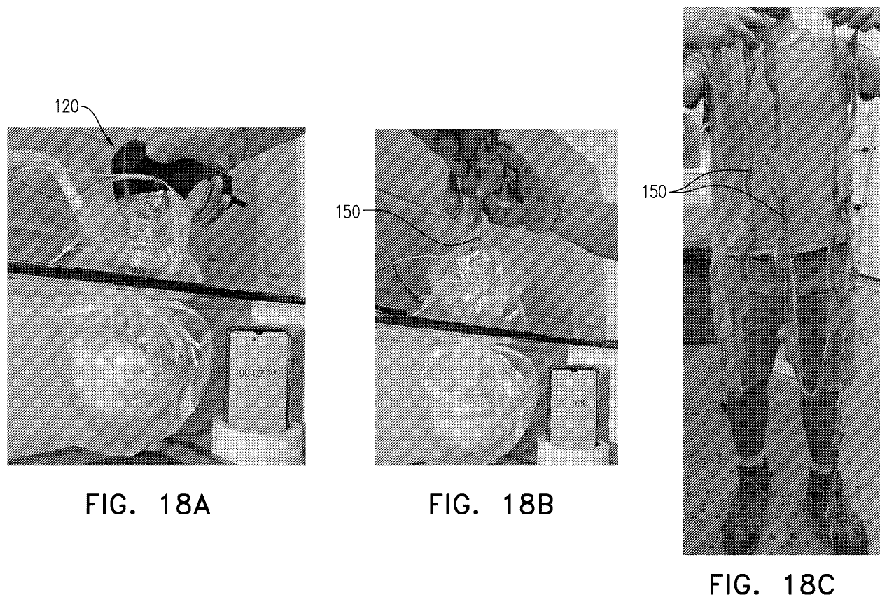

As shown in FIGS. 16D-E, resected tissue specimen 22 is inserted into retrieval bag 30 via opening 32, while retrieval bag 30 is in the patient's body 24. Although in practice resected tissue specimen 22 typically touches inner surface 48 of retrieval bag 30, in FIGS. 16E-H (and FIG. 18) a small gap is shown between resected tissue specimen 22 and inner surface 48 for clarity of illustration.

Optionally, retrieval bag 30 comprises a wire (e.g., comprising a shape memory alloy, such as Nitinol) around opening 32, to help open the bag.

As shown in FIG. 16E, proximal portion 35 of retrieval bag 30 (and a proximal portion of containment bag 80, if provided) is pulled out of the body via laparoscopic port 43.

As shown in FIG. 16F, insertion tip 130 of control handle 120 is optionally inserted into retrieval bag 30, typically until the tip comes in physical contact with resected tissue specimen 22.

As shown in the transition between FIG. 16F and FIG. 16G, pulling segment 40 of electrosurgical slicing wire 34 is pulled, during application of RF electrosurgical power between electrosurgical slicing wire 34 and return electrode 42 when resected tissue specimen 22 is within retrieval bag 30 and return electrode 42 is in electrical contact with resected tissue specimen 22, thereby detaching successive longitudinal portions of slicing segment 36 of electrosurgical slicing wire 34 from wall 38 of retrieval bag 30 and slicing resected tissue specimen 22 within retrieval bag 30 into one or more pieces 150 of tissue. The physical contact between tip 130 and resected tissue specimen 22 directs the most recently detached portion of slicing segment 36 through resected tissue specimen 22 to slice the resected tissue specimen, and prevents the most recently detached portion of slicing segment 36 from traveling around the tissue specimen rather than through the tissue specimen. RF ESG 26 is typically activated in monopolar mode.

Typically, resected tissue specimen 22 generally maintains its original shape upon slicing, until the one or more sliced pieces 150 of tissue are removed from retrieval bag 30, as described immediately below.

As shown in FIG. 16H-I, while retrieval bag 30 is partially within the patient's body 24 such that opening 32 of retrieval bag 30 is outside the patient's body 24, the one or more pieces 150 of tissue are removed from retrieval bag 30 (via opening 32) and the patient's body 24 via laparoscopic port 43, such as using grasper 146, as is known in the tissue removal art. Optionally, the one or more pieces of 150 have a shape similar to that illustrated in FIG. 17B, described hereinbelow. As mentioned above, slicing resected tissue specimen 22 into a relatively small number of pieces 150, ideally a single piece 150 when possible, reduces the number of pieces that must be separately removed from retrieval bag 30 during the tissue removal procedure. This may substantially shorten the length of the tissue removal procedure. For vaginal procedures, the one or more pieces 150 of tissue are removed via the vaginal port.

FIG. 16J shows the patient's body 24 after retrieval bag 30 (and containment bag 80, if provided) has been removed from the patient's body 24.

For some applications, pulling pulling segment 40 of electrosurgical slicing wire 34, as shown in FIGS. 16H-I, slices resected tissue specimen 22 within retrieval bag 30:
into ten or fewer pieces 150 of tissue, such as five or fewer pieces 150, e.g., three or fewer pieces 150, such as a single piece 150 of tissue, such as shown and labeled in FIGS. 16F-I; and/or
into one or more pieces 150 of tissue, at least one of which has a length greater than a multiple of the greatest thickness T of the piece 150 of tissue perpendicular to the length (at the location along the piece 150 having the greatest thickness T), the multiple equal to 10, such as equal to 20, such as shown and labeled in in FIG. 16I.

For some applications, tissue removal apparatus 20 is configured to slice resected tissue specimen 22 such that the one or more pieces 150 of tissue collectively have an average thickness of no more than 1 cm, such as 0.5-1 cm.

As described hereinabove with reference to FIGS. 4A-B and 5, for some applications, tissue removal apparatus 20 further comprises containment bag 80 within which retrieval bag 30 is disposed, so as to define one or more inflatable chambers 82 between inner surface 84 of containment bag 80 and outer surface 86 of retrieval bag 30. For these applications, containment bag 80 is also inserted into the patient's body 24, typically at the same time as retrieval bag 30 (while retrieval bag 30 is within containment bag 80). The method may further comprise, after inserting retrieval bag 30 and containment bag 80 into the patient's body 24, inflating the one or more inflatable chambers 82 with a fluid, such as a gas (e.g., air, such as clean dry air (CDA) or CO2, or a liquid (e.g., saline solution)).

As described hereinabove with reference to FIGS. 4A-B and 5, for some applications, wall 90 of containment bag 80 is shaped so as to define a plurality of inflatable compartments 92. For these applications, the method may further comprise, after inserting retrieval bag 30 and containment bag 80 into the patient's body 24, inflating the plurality of inflatable compartments 92 with a fluid, such as a gas (e.g., air, such as clean dry air (CDA) or CO2, or a liquid (e.g., saline solution)).

As described hereinabove with reference to FIGS. 4A-B and 5, for some applications, containment bag 80 includes camera port 96 open to one of the one or more inflatable chambers 82. For these applications, the method may further comprise inserting a camera 148 through camera port 96, and viewing retrieval bag 30 from within containment bag 80 (as labeled in FIG. 16I).

Reference is again made to FIG. 16E. In an application of the present invention, tissue removal system 10 further comprises a conductive gel 160 for filling at least a portion of empty spaces 162 between resected tissue specimen 22 and inner surface 48 of retrieval bag 30 when resected tissue specimen 22 is within retrieval bag 30. Typically, the portion of empty spaces 162 only alongside resected tissue specimen 22 and slicing segment 36 of electrosurgical slicing wire 34 are filled with conductive gel 160 (for example, retrieval bag 30 may be marked with a predetermined marking line slightly above the proximal-most point of slicing segment 36). Conductive gel 160 may help prevent retrieval bag 30 from folding into empty spaces 162 and/or prevent slicing segment 36 of electrosurgical slicing wire 34 from prematurely detaching from wall 38 of retrieval bag 30.

Typically, conductive gel 160 is viscous.

In an application of the present invention, a tissue removal system 10 is provided that comprises:
a tissue removal apparatus 20 comprising:
retrieval bag 30 having opening 32 for receiving resected tissue specimen 22 within the patient's body 24; and
one or more tissue cutters detachably coupled to wall 38 of retrieval bag 30, which are configured to cut resected tissue specimen 22 when resected tissue specimen 22 is within retrieval bag 30; and
conductive gel 160 for filling at least a portion of empty spaces 162 between resected tissue specimen 22 and inner surface 48 of retrieval bag 30 when resected tissue specimen 22 is within retrieval bag 30.

For some applications, the one or more tissue cutters comprise one or more cutting wires, which may either not be electrically couplable to a generator or may be coupled electrically couplable to a generator, in which case tissue removal apparatus 20 typically further comprises one or more electrical leads for coupling the one or more tissue cutters to the generator. For example, the one or more tissue cutters may comprise slicing segment 36 of electrosurgical slicing wire 34, as described above. Alternatively, the one or more tissue cutters comprise a different type of tissue cutter, such as one or more blades.

Reference is made to FIGS. 17A-B, which are schematic illustrations of a portion of a method for removing resected tissue specimen 22 from body 24 of a patient, in accordance with an application of the present invention. The steps of the method illustrated in FIGS. 17A-B generally correspond to the steps of the method illustrated above in FIGS. 16E-I, and may be performed in combination with these and other steps of the method illustrated in FIGS. 16A-J. FIGS. 17A-B illustrate the configuration of retrieval bag 30 described hereinabove with reference to FIGS. 6A-B, and the configuration of retrieval bag 30 described hereinabove with reference to FIG. 9.

Reference is now made to FIGS. 18A-C, which are photographs of an experiment conducted by some of the inventors to test certain elements of the apparatus and methods described herein. A beef tongue having a mass of about 700 g was used as the tissue specimen. A tissue removal system was used that was similar in many respects to tissue removal system 10, described herein. The tissue removal system comprised a tissue removal apparatus similar to tissue removal apparatus 20 described hereinabove with reference to FIGS. 4A-B and 5, except that the containment bag did not define any inflatable compartments. The retrieval bag was disposed within the containment bag so as to define an inflatable chamber between the inner surface of the containment bag and the outer surface of the retrieval bag. The inflatable chamber between the inner and outer bags was inflated with clean dry air (CDA).

The experiment used an RF electrosurgical generator (ESG) configured in monopolar mode with a frequency of 99-460 kHz, with power up to 350 W, and the power level set to 7 out of 11 (where setting 11=350 W). A control handle similar to control handle 120, described hereinabove with reference to FIG. 15A was used to pull the electrosurgical slicing wire with a predefined pulling force limit. The handle was coupled to a smoke evacuation system (OBS® Surgical Smoke Evacuator SS-100 510, manufactured by Baisheng Medical Co., Ltd., Jiangmen, Guangdong, China). The retrieval bag comprised an integrated return electrode, which was electrically coupled to a return electrode receptacle of the RF ESG. The electrosurgical slicing wire comprised tungsten and had a diameter of 0.09 mm; both ends of the electrosurgical slicing wire were electrically coupled to a monopolar active receptacle of the RF ESG.

During the experiment, the tissue specimen was successfully sliced inside the retrieval bag by inserting the tip of the control handle into the retrieval bag via a 15 mm hole simulating a laparoscopic port. The tissue slicing had a duration of about 100 seconds. A tissue grasper was used to extract the sliced tissue from the retrieval bag via the same 15 mm hole. The grasper first removed two small pieces of tissue, and the remainder of the tissue in a single long piece of tissue having a length of about 5 meters, then all in one long piece The duration of the tissue extraction from the bag was about 100 seconds.

This experiment demonstrated that techniques similar to those described herein successfully quickly sliced a tissue specimen with a retrieval bag into a small number (three) of pieces, and quickly extracted the pieces from the retrieval bag.

Although some configurations of the present invention have been described herein as applying RF energy using electrosurgical slicing wire 34, in other configurations, RF energy is not applied; for example, the wire may instead by heated, such as using Ohmic heating, so as to perform electrocautery, or the wire may slice the tissue using only mechanical forces.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A tissue removal apparatus for removing a resected tissue specimen from a body of a patient, the tissue removal apparatus for use with an RF electrosurgical generator, the tissue removal apparatus comprising:
a retrieval bag having an opening for receiving the resected tissue specimen within the patient's body, wherein the retrieval bag is flexible and is configured to assume compressed and expanded configurations;
an electrosurgical slicing wire that includes a slicing segment detachably coupled to a wall of the retrieval bag, and a pulling segment free of the wall of the retrieval bag and extending out of the opening of the retrieval bag; and
a return electrode,
wherein when the retrieval bag is in the expanded configuration:
the retrieval bag defines a central longitudinal axis, and
the slicing segment of the electrosurgical slicing wire winds around the central longitudinal axis at varying locations along the central longitudinal axis, and
wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.

2. The apparatus according to claim 1, wherein the tissue removal apparatus comprises exactly one electrosurgical slicing wire, which is single-stranded or multi-stranded.

3. The apparatus according to claim 1, wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment of the electrosurgical slicing wire from the wall of the retrieval bag and slices the resected tissue specimen within the retrieval bag into ten or fewer pieces of tissue.

4. A tissue removal apparatus for removing a resected tissue specimen from a body of a patient, the tissue removal apparatus for use with an RF electrosurgical generator, the tissue removal apparatus comprising:
a retrieval bag having an opening for receiving the resected tissue specimen within the patient's body;
exactly one electrosurgical slicing wire that includes a slicing segment detachably coupled to a wall of the retrieval bag, and a pulling segment free of the wall of the retrieval bag and extending out of the opening of the retrieval bag, wherein the wire is single-stranded or multi-stranded; and
a return electrode,
wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag.

5. The apparatus according to claim 4, wherein the wire is multi-stranded and braided.

6. The apparatus according to claim 4, wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment of the electrosurgical slicing wire from the wall of the retrieval bag and slices the resected tissue specimen within the retrieval bag into ten or fewer pieces of tissue.

7. A tissue removal apparatus for removing a resected tissue specimen from a body of a patient, the tissue removal apparatus for use with an RF electrosurgical generator, the tissue removal apparatus comprising:
a retrieval bag having an opening for receiving the resected tissue specimen within the patient's body;
an electrosurgical slicing wire that includes an electrically-insulated slicing segment detachably coupled to a wall of the retrieval bag, and a pulling segment free of the wall of the retrieval bag and extending out of the opening of the retrieval bag; and
a return electrode,
wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment from the wall of the retrieval bag, electrically exposes the successive longitudinal portions as they are detached from the wall, and slices the resected tissue specimen within the retrieval bag.

8. The apparatus according to claim 7, wherein the tissue removal apparatus comprises exactly one electrosurgical slicing wire, which is single-stranded or multi-stranded.

9. The apparatus according to claim 7, wherein the tissue removal apparatus is configured such that the pulling of the pulling segment of the electrosurgical slicing wire, during the application of RF electrosurgical power between the electrosurgical slicing wire and the return electrode, detaches the successive longitudinal portions of the slicing segment of the electrosurgical slicing wire from the wall of the retrieval bag and slices the resected tissue specimen within the retrieval bag into ten or fewer pieces of tissue.

10. The apparatus according to claim 7 9, wherein the slicing segment of the electrosurgical slicing wire is detachably embedded in the wall of the retrieval bag, such that the wall electrically insulates the slicing segment of the electrosurgical slicing wire.

11. A tissue removal apparatus for removing a resected tissue specimen from a body of a patient, the tissue removal apparatus for use with an RF electrosurgical generator, the tissue removal apparatus comprising:
a retrieval bag having an opening for receiving the resected tissue specimen within the patient's body;
an electrosurgical slicing wire that includes a slicing segment detachably coupled to a wall of the retrieval bag, and a pulling segment free of the wall of the retrieval bag and extending out of the opening of the retrieval bag; and
a return electrode,
wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag into ten or fewer pieces of tissue.

12. The apparatus according to claim 11, wherein the tissue removal apparatus is configured to slice the resected tissue specimen into five or fewer pieces of tissue.

13. The apparatus according to claim 11, wherein the tissue removal apparatus is configured to slice the resected tissue specimen such that at least one of the pieces of tissue has a length greater than a multiple of a greatest thickness of the piece of tissue perpendicular to the length, the multiple equal to 10.

14. A tissue removal apparatus for removing a resected tissue specimen from a body of a patient, the tissue removal apparatus for use with an RF electrosurgical generator, the tissue removal apparatus comprising:
a retrieval bag having an opening for receiving the resected tissue specimen within the patient's body;
an electrosurgical slicing wire that includes a slicing segment detachably coupled to a wall of the retrieval bag, and a pulling segment free of the wall of the retrieval bag and extending out of the opening of the retrieval bag; and
a return electrode,
wherein the tissue removal apparatus is configured such that pulling the pulling segment of the electrosurgical slicing wire, during application of RF electrosurgical power between the electrosurgical slicing wire and the return electrode when the resected tissue specimen is within the retrieval bag and the return electrode is in electrical contact with the resected tissue specimen, detaches successive longitudinal portions of the slicing segment of the electrosurgical slicing wire from the wall of the retrieval bag and slices the resected tissue specimen within the retrieval bag into one or more pieces of tissue, at least one of which has a length greater than a multiple of a greatest thickness of the piece of tissue perpendicular to the length, the multiple equal to 10.

15. The apparatus according to claim 14, wherein the multiple is equal to 20.

16-68. (canceled)

69. The apparatus according to claim 3, wherein the tissue removal apparatus is configured to slice the resected tissue specimen into five or fewer pieces of tissue.

70. The apparatus according to claim 6, wherein the tissue removal apparatus is configured to slice the resected tissue specimen into five or fewer pieces of tissue.

71. The apparatus according to claim 8, wherein the slicing segment of the electrosurgical slicing wire is detachably embedded in the wall of the retrieval bag, such that the wall electrically insulates the slicing segment of the electrosurgical slicing wire.

72. The apparatus according to claim 9, wherein the tissue removal apparatus is configured to slice the resected tissue specimen into five or fewer pieces of tissue.

73. The apparatus according to claim 9, wherein the slicing segment of the electrosurgical slicing wire is detachably embedded in the wall of the retrieval bag, such that the wall electrically insulates the slicing segment of the electrosurgical slicing wire.
